(19) United States
(12) Patent Application Publication
(10) Pub. No.: US 2026/0306638 A1
(43) Pub. Date: Oct. 8, 2026

(54) COMPOUNDS AND USES THEREOF

(71) Applicant: FOGHORN THERAPEUTICS INC., Cambridge, MA (US)

(72) Inventors: Matthew NETHERTON, Cambridge, MA (US); Francois BRUCELLE, Cambridge, MA (US); Jing DENG, Cambridge, MA (US)

(21) Appl. No.: 19/478,172

(22) PCT Filed: May 9, 2024
(86) PCT No.: PCT/US2024/028458

§ 371 Date: Oct. 23, 2025

Related U.S. Application Data
(60) Provisional application No. 63/501,267, filed on May 10, 2023.

(51) Int. Cl. A61K 47/54

(52) U.S. Cl. CPC A61K 47/545

(57) ABSTRACT

The present disclosure features compounds of Formula I, or pharmaceutically acceptable salts thereof, and formulations containing the same which are useful for modulating BRG1- or BRM-associated factors (BAF) complexes. Methods of treating BAF complex-related disorders, such as cancer, are also disclosed.

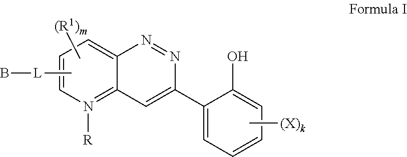

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In an aspect, the invention features a compound, or a pharmaceutically acceptable salt thereof, having the structure of Formula I:

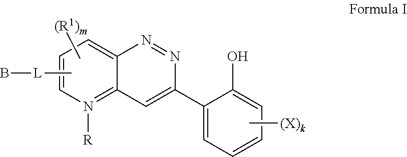

where
m is 0, 1, 2, or 3;
k is 0, 1, or 2;
R is absent or optionally substituted C₁-C₆ alkyl;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl, optionally substituted C₃-C₈ cycloalkoxy, optionally substituted C₂-C₆ alkynyl, optionally substituted amino, or cyano;
each X is, independently, halo or optionally substituted C₁-C₆ heteroalkyl;
L is a linker; and
B is a degradation moiety.

In some embodiments, the compound has the structure of Formula I-A:

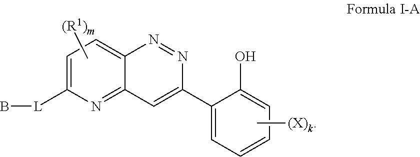

In some embodiments, the compound has the structure of Formula I-B

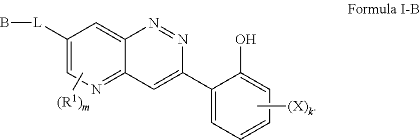

In some embodiments, the compound has the structure of Formula I-C:

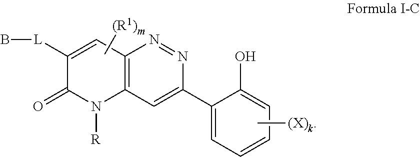

In some embodiments, m is 0. In some embodiments, in is 1. In some embodiments, m is 2. In some embodiments, in is 3.

In some embodiments, R¹ is optionally substituted C₁-C₆ heteroalkyl. In some embodiments, R¹ is alkoxy. In some embodiments, R¹ is methoxy. In some embodiments, R¹ is halo. In some embodiments, R¹ is F or Cl. In some embodiments, R¹ is optionally substituted C₁-C₆ alkyl. In some embodiments, R¹ is methyl. In some embodiments, R¹ is difluoromethoxy. In some embodiments, R¹ is difluoromethyl. In some embodiments, R¹ is optionally substituted C₂-C₆ alkynyl. In some embodiments, R¹ is methyne. In some embodiments, R¹ is optionally substituted C₃-C₈ cycloalkyl. In some embodiments, R¹ is cyclopropane. In some embodiments, R¹ is cyclopropoxy. In some embodiments, R¹ is optionally substituted C₂-C₉ heterocyclyl. In some embodiments, R¹ is optionally substituted amino. In some embodiments, R¹ is cyano.

In some embodiments, k is 0. In some embodiments, k is 1. In some embodiments, k is 2. In some embodiments, X is optionally substituted C₁-C₆ heteroalkyl. In some embodiments, X is methoxy. In some embodiments, X is halo. In some embodiments, X is F.

In some embodiments, the degradation moiety, B, has the structure of Formula A-1:

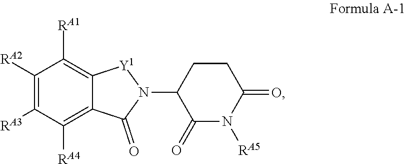

where
Y¹ is

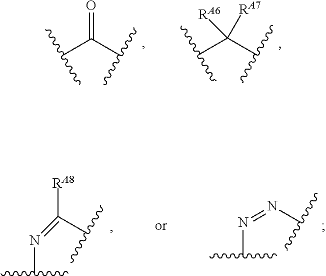

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7} together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{4A}, together with the carbon atoms to which each is attached, combine to form

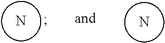

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
   where one of R^{A1}, R^{A2}, R^{A3}, and R^{A4} is A², or

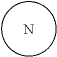

is substituted with A²; and
A² is a bond between the degradation moiety and the linker.

In some embodiments, R^{A5} is H or methyl. In some embodiments, R^{A5} is H.

In some embodiments, each of R^{A1}, R^{A2}, R^{A3}, and R^{A4} is, independently, H or A².

In some embodiments, R^{A1} is A² and each of R^{A2}, R^{A3}, and R^{A4} is H.

In some embodiments, R^{A2} is A² and each of R^{A1}, R^{A3}, and R^{A4} is H.

In some embodiments, R^{A3} is A² and each of R^{A1}, R^{A2}, and R^{A4} is H.

In some embodiments, R^{A4} is A² and each of R^{A1}, R^{A2}, and R^{A3} is H.

In some embodiments, Y¹ is

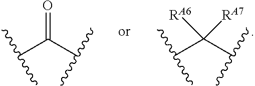

In some embodiments, R^{A6} is H. In some embodiments, R^{A7}, is H.

In some embodiments, Y¹ is

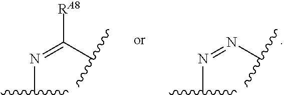

In some embodiments, R^{A8} is H or optionally substituted C₁-C₆ alkyl. In some embodiments. R^{A8} is H or methyl. In some embodiments, R^{A8} is methyl.

In some embodiments, the degradation moiety includes the structure of Formula A2:

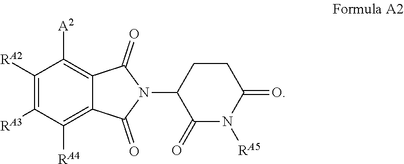

In some embodiments, the degradation moiety is

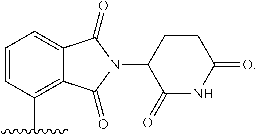 [SMILES: *c1cccc2c1C(=O)N(C1CCC(=O)NC1=O)C2=O]

In some embodiments, the degradation moiety includes the structure of Formula A4:

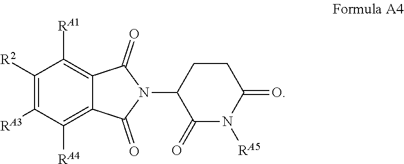

In some embodiments, the degradation moiety is

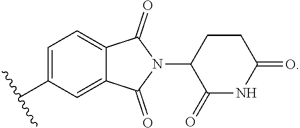 [SMILES: *c1ccc2c(c1)C(=O)N(C1CCC(=O)NC1=O)C2=O]

In some embodiments, the degradation moiety has the structure of Formula A5:

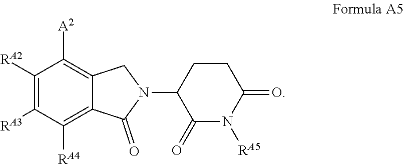

In some embodiments, the degradation moiety has the structure of Formula A6:

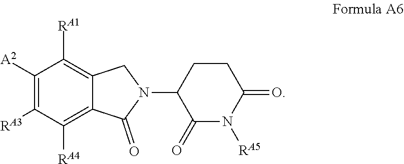

In some embodiments, the degradation moiety has the structure of Formula A8:

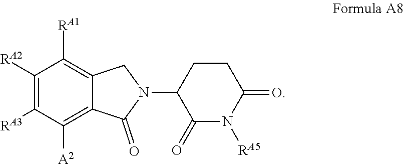

In some embodiments, the degradation moiety has the structure of Formula A10:

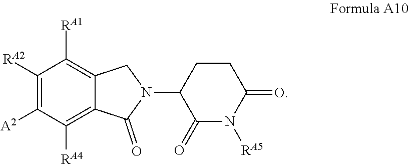

In some embodiments the degradation moiety has the structure of

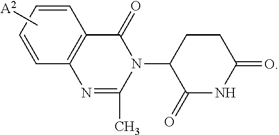

In some embodiments, the degradation moiety has the structure of

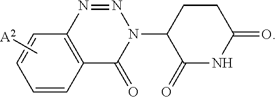

In some embodiments, the degradation moiety has the structure of Formula C:

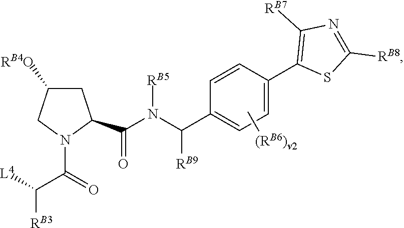

where
L⁴ is —N(R^{B1})(R^{B2}),

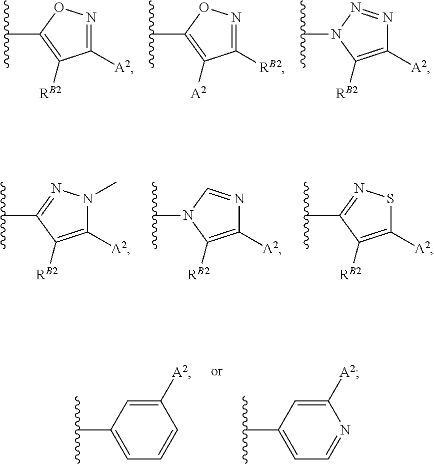

R^{B1} is H, optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
where one and only one of R^{B1}, R^{B3}, and R^{B6} is A²;
or a pharmaceutically acceptable salt thereof.

In some embodiments, the degradation moiety has the structure of Formula C:

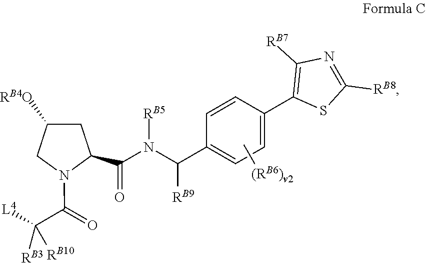

where
L⁴ is —N(R^{B1})(R^{B2}),

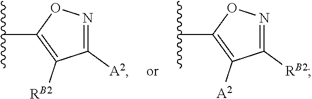

R^{B1} is H, A², optionally substituted C₁-C₆ alkyl or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B10} is H or F; and
A² is a bond between the degradation moiety and the linker;
where one and only one of R^{B1}, R^{B3}, and R^{B6} is A²,
   or a pharmaceutically acceptable salt thereof.

In some embodiments, the degradation moiety has the structure of Formula C3.

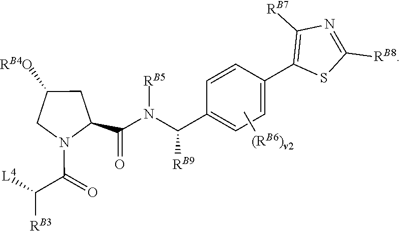

In some embodiments, the degradation moiety has the structure of Formula C4.

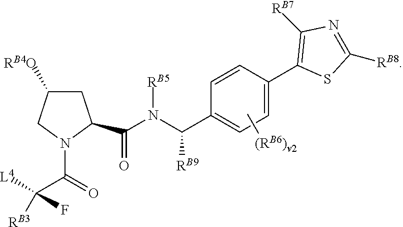

In some embodiments, the degradation moiety has the structure of Formula C1:

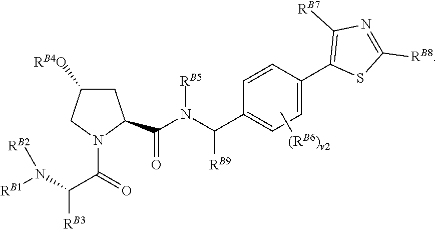

In some embodiments, the degradation moiety is

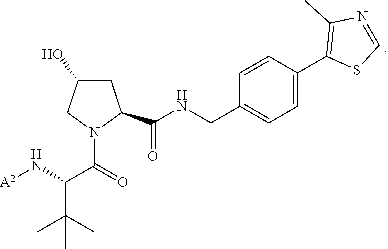

In some embodiments, the degradation moiety is

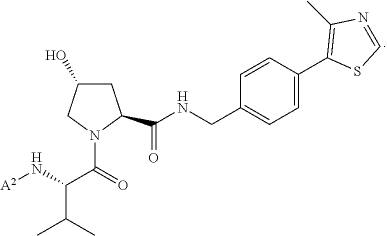

In some embodiments, the degradation moiety is

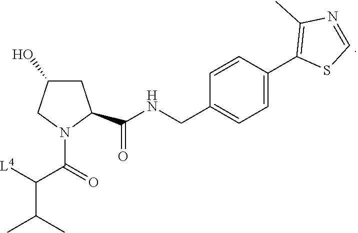

In some embodiments, the degradation moiety is

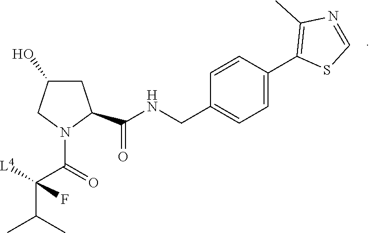

In some embodiments, the degradation moiety is

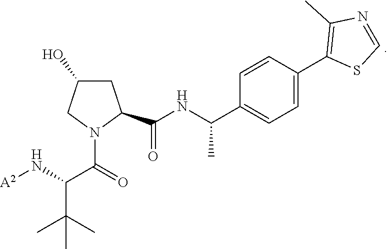

In some embodiments the degradation moiety is

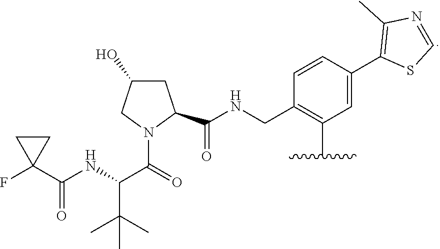 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, the degradation moiety has the structure of Formula C2:

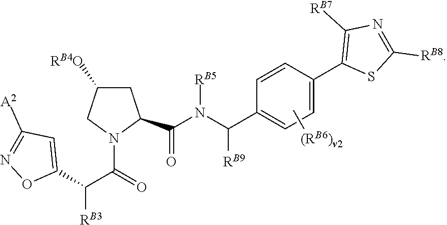

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B4} is H. In some embodiments, R^{B5} is H. In some embodiments, R^{B7} is optionally substituted C₁-C₆ alkyl. In some embodiments, R^{B7} is methyl. In some embodiments, R^{B3} is optionally substituted C₁-C₆ alkyl. In some embodiments, R^{B3} is isopropyl. In some embodiments, R^{B8} is H. In some embodiments, R^{B2} is H.

In some embodiments, the degradation moiety is

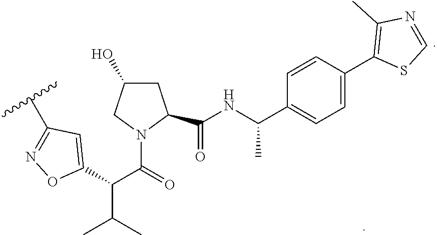 [SMILES: *c1cc([C@H](C(=O)N2C[C@H](O)C[C@H]2C(=O)N[C@@H](C)c2ccc(-c3scnc3C)cc2)C(C)C)on1]

In some embodiments, the degradation moiety has the structure of Formula Ca2:

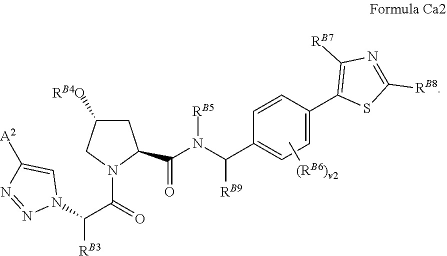

In some embodiments, the degradation moiety has the structure of Formula Cb2:

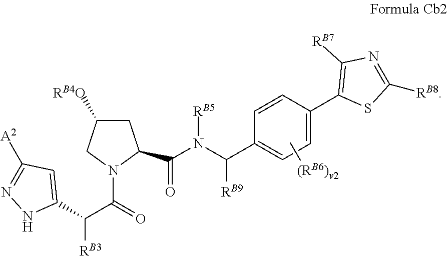

In some embodiments, the degradation moiety has the structure of Formula Cc2:

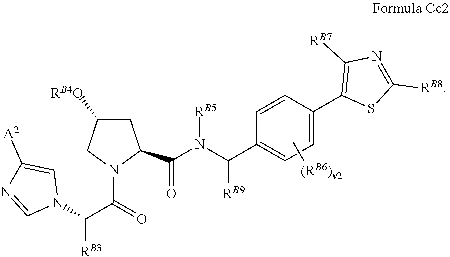

In some embodiments, the degradation moiety has the structure of Formula Cd2:

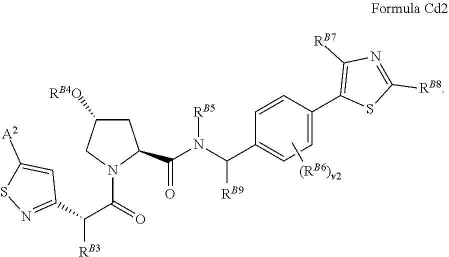

In some embodiments, the degradation moiety has the structure of Formula Ce2:

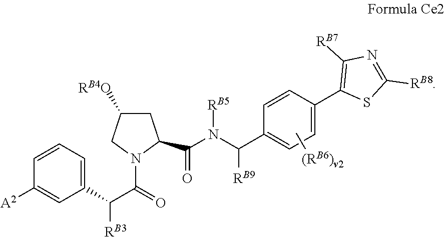

In some embodiments, the degradation moiety has the structure of Formula Cf2:

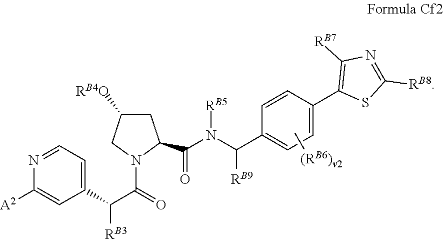

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B4} is H. In some embodiments, R^{B5} is H. In some embodiments, R^{B7} is optionally substituted C₁-C₆ alkyl. In some embodiments, R^{B7} is methyl. In some embodiments, R^{B3} is optionally substituted C₁-C₆ alkyl. In some embodiments, R^{B3} is isopropyl. In some embodiments, R^{B3} is optionally substituted C₃-C₁₀ carbocyclyl. In some embodiments, R^{B3} is cyclopropane. In some embodiments, R^{B3} is cyclobutane. In some embodiments. R^{B3} is fluoro-2-methylpropane. In some embodiments, R^{B8} is H. In some embodiments. R^{B2} is H.

In some embodiments, the degradation moiety is

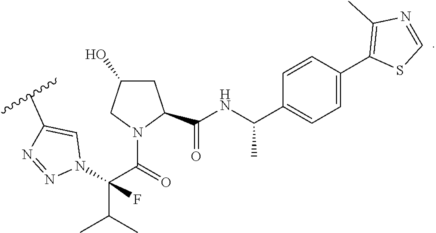

In some embodiments, the degradation moiety is

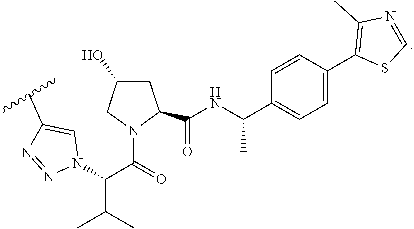 [SMILES: *c1cn([C@H](C(=O)N2C[C@H](O)C[C@H]2C(=O)N[C@@H](C)c2ccc(-c3scnc3C)cc2)C(C)C)nn1]

In some embodiments, the degradation moiety is

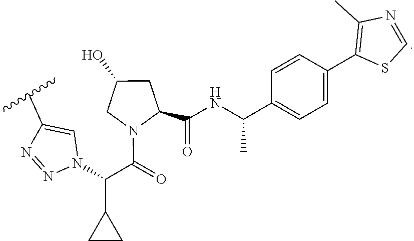 [SMILES: *c1cn([C@H](C(=O)N2C[C@H](O)C[C@H]2C(=O)N[C@@H](C)c2ccc(-c3scnc3C)cc2)C2CC2)nn1]

In some embodiments, the degradation moiety is

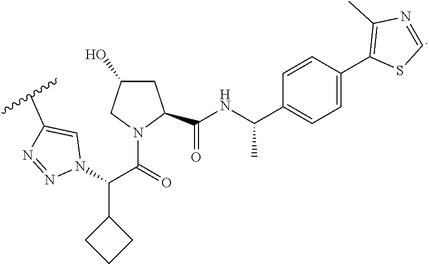 [SMILES: *c1cn([C@H](C(=O)N2C[C@H](O)C[C@H]2C(=O)N[C@@H](C)c2ccc(-c3scnc3C)cc2)C2CCC2)nn1]

In some embodiments, the degradation moiety is

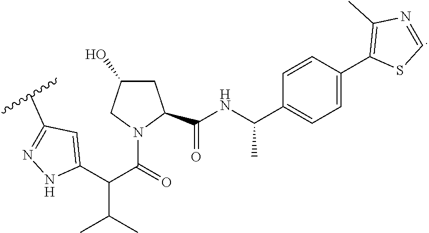 [SMILES: *c1cc(C(C(=O)N2C[C@H](O)C[C@H]2C(=O)N[C@@H](C)c2ccc(-c3scnc3C)cc2)C(C)C)[nH]n1]

In some embodiments, the degradation moiety is

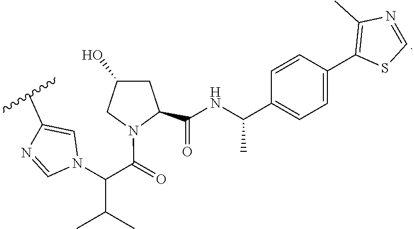 [SMILES: *c1cn(C(C(=O)N2C[C@H](O)C[C@H]2C(=O)N[C@@H](C)c2ccc(-c3scnc3C)cc2)C(C)C)cn1]

In some embodiments, the degradation moiety is

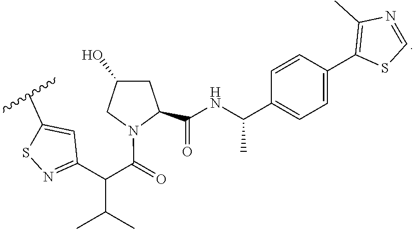 [SMILES: *c1cc(C(C(=O)N2C[C@H](O)C[C@H]2C(=O)N[C@@H](C)c2ccc(-c3scnc3C)cc2)C(C)C)ns1]

In some embodiments, the degradation moiety is

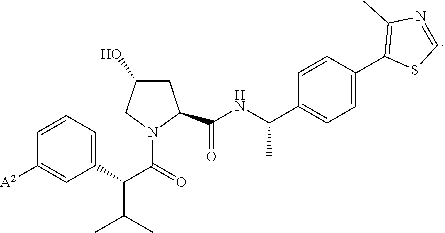

In some embodiments, the degradation moiety is

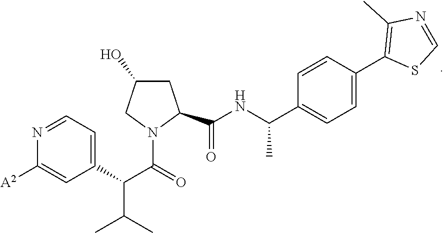

In some embodiments, the degradation moiety as the structure of Formula C5:

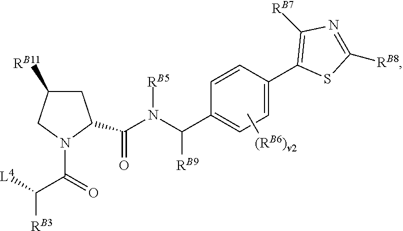

where
L⁴ is —N(R^{B1})(R^{B2}),

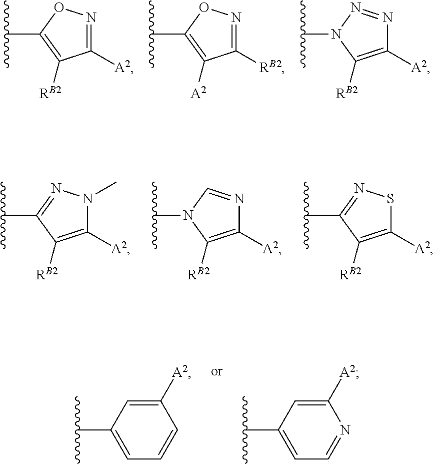

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl,
v2 is 0, 1, 2, 3, or 4;
each R^{B6} is, independently, A², halogen, optionally substituted C₃-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B11} is H, alcohol, boronic acid, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; and
A² is a bond between the degradation moiety and the linker;
where one and only one of R^{B1}, R^{B3}, and R^{B6} is A²,
or a pharmaceutically acceptable salt thereof.

In some embodiments, R^{B11} is boric acid.

In some embodiments, the degradation moiety has the structure of Formula C6.

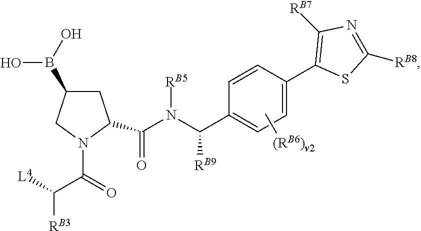

In some embodiments, the degradation moiety has the structure of Formula C1:

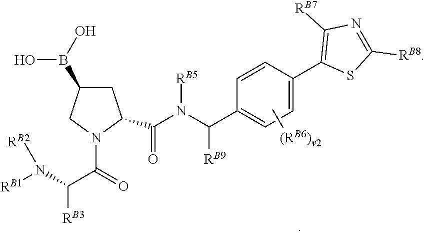

In some embodiments, the degradation moiety has the structure of Formula C8:

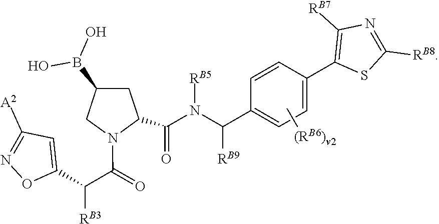

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B5} is H. In some embodiments, R^{B7} is optionally substituted C₁-C₆ alkyl. In some embodiments, Reis methyl. In some embodiments, R^{B3} is optionally substituted C₁-C₆ alkyl. In some embodiments, R^{B3} is isopropyl. In some embodiments. R^{B8} is H. In some embodiments, R^{B2} is H.

In some embodiments, the degradation moiety is

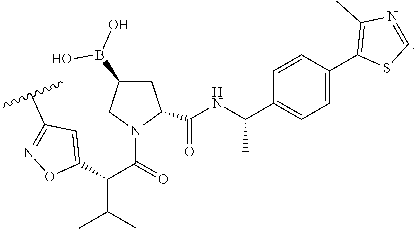 [SMILES: *c1cc([C@H](C(=O)N2C[C@@H](B(O)O)C[C@@H]2C(=O)N[C@@H](C)c2ccc(-c3scnc3C)cc2)C(C)C)on1]

In some embodiments, the degradation moiety has the structure of Formula D:

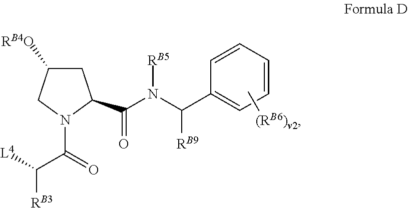

where
L⁴ is —N(R^{B1})(R^{B2}),

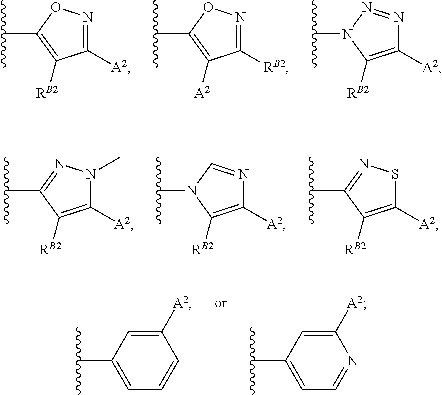

R^{B1} is H, A², optionally substituted C₁-C₆ alkyl, or optionally substituted C₁-C₆ heteroalkyl,
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, i, 2, 3, or 4;
each R^{B6} is, independently, A², halogen, optionally substituted C₁-C₆ alkyl, optionally substituted C₁-C₆ heteroalkyl, optionally substituted C₂-C₆ alkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula D3.

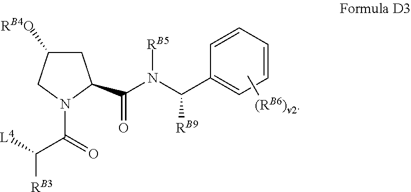

In some embodiments, the degradation moiety has the structure of Formula D1:

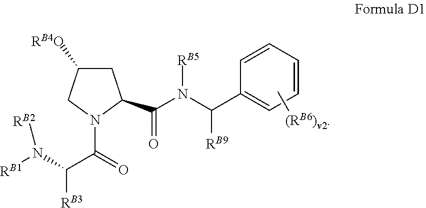

In some embodiments, the degradation moiety is

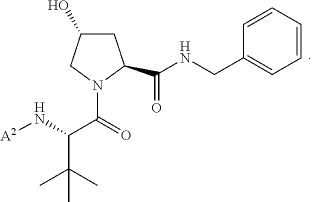

In some embodiments, the degradation moiety is

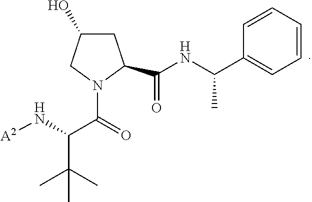

In some embodiments, the degradation moiety is

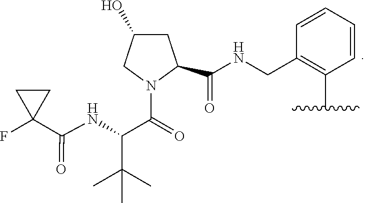 [SMILES: *c1ccccc1CNC(=O)[C@@H]1C[C@@H](O)CN1C(=O)[C@@H](NC(=O)C1(F)CC1)C(C)(C)C]

In some embodiments, the degradation moiety has the structure of Formula D2:

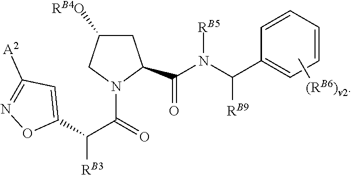

In some embodiments, R^{B9} is optionally substituted C₁-C₆ alkyl. In some embodiments, R^{B9} is methyl.

In some embodiments, R^{B9} is bonded to (S)-stereogenic center. In some embodiments, R^{B9} is H.

In some embodiments, v2 is 0. In some embodiments, v2 is 1. In some embodiments, v2 is 2. In some embodiments, R^{B4} is H. In some embodiments, R⁵ is H. In some embodiments, R^{B3} is optionally substituted C₁-C₆ alkyl. In some embodiments, R^{B3} is isopropyl. In some embodiments, R^{B6} is H. In some embodiments, R^{B6} is halogen. In some embodiments, R^{B6} is fluorine. In some embodiments, R^{B6} is bromine. In some embodiments, R^{B6} is chlorine. In some embodiments, R^{B6} is cyano. In some embodiments, R^{B6} is optionally substituted C₁-C₆ heteroalkyl. In some embodiments, R^{B6} is optionally substituted C₃-C₆ alkynyl. In some embodiments, R^{B6} is methoxy. In some embodiments, R^{B6} is 3-methoxy-1-propanoxy.

In some embodiments, the degradation moiety is

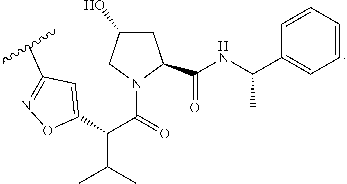 [SMILES: *c1cc([C@H](C(=O)N2C[C@H](O)C[C@H]2C(=O)N[C@@H](C)c2ccccc2)C(C)C)on1]

In some embodiments, the degradation moiety is

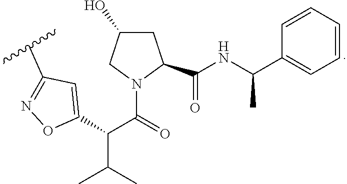 [SMILES: *c1cc([C@H](C(=O)N2C[C@H](O)C[C@H]2C(=O)N[C@H](C)c2ccccc2)C(C)C)on1]

In some embodiments, the degradation moiety is

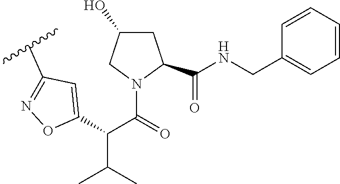 [SMILES: *c1cc([C@H](C(=O)N2C[C@H](O)C[C@H]2C(=O)NCc2ccccc2)C(C)C)on1]

In some embodiments, the degradation moiety is

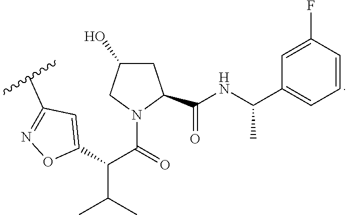 [SMILES: *c1cc([C@H](C(=O)N2C[C@H](O)C[C@H]2C(=O)N[C@@H](C)c2cccc(F)c2)C(C)C)on1]

In some embodiments, the degradation moiety is

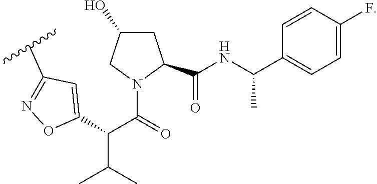 [SMILES: *c1cc([C@H](C(=O)N2C[C@H](O)C[C@H]2C(=O)N[C@@H](C)c2ccc(F)cc2)C(C)C)on1]

In some embodiments, the degradation moiety is

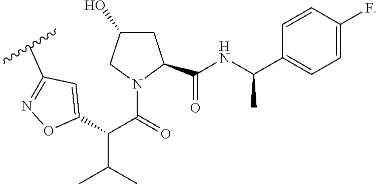 [SMILES: *c1cc([C@H](C(=O)N2C[C@H](O)C[C@H]2C(=O)N[C@H](C)c2ccc(F)cc2)C(C)C)on1]

In some embodiments, the degradation moiety is

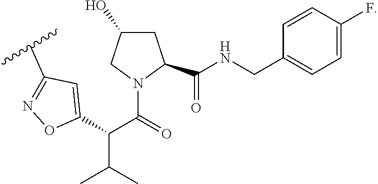 [SMILES: *c1cc([C@H](C(=O)N2C[C@H](O)C[C@H]2C(=O)NCc2ccc(F)cc2)C(C)C)on1]

In some embodiments, the degradation moiety is

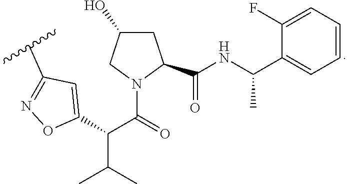 [SMILES: *c1cc([C@H](C(=O)N2C[C@H](O)C[C@H]2C(=O)N[C@@H](C)c2ccccc2F)C(C)C)on1]

In some embodiments, the degradation moiety is

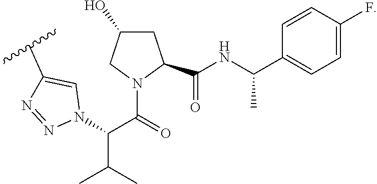 [SMILES: *c1cn([C@H](C(=O)N2C[C@H](O)C[C@H]2C(=O)N[C@@H](C)c2ccc(F)cc2)C(C)C)nn1]

In some embodiments, the degradation moiety is

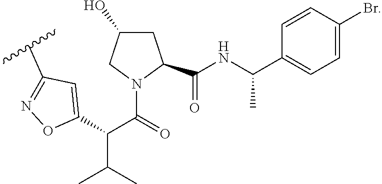 [SMILES: *c1cc([C@H](C(=O)N2C[C@H](O)C[C@H]2C(=O)N[C@@H](C)c2ccc(Br)cc2)C(C)C)on1]

In some embodiments, the degradation moiety is

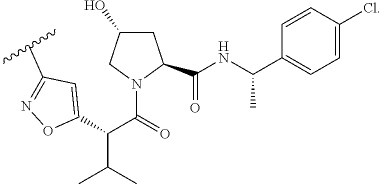 [SMILES: *c1cc([C@H](C(=O)N2C[C@H](O)C[C@H]2C(=O)N[C@@H](C)c2ccc(Cl)cc2)C(C)C)on1]

In some embodiments, the degradation moiety is

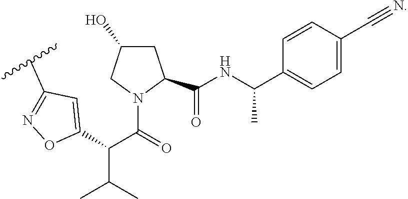 [SMILES: *c1cc([C@H](C(=O)N2C[C@H](O)C[C@H]2C(=O)N[C@@H](C)c2ccc(C#N)cc2)C(C)C)on1]

In some embodiments, the degradation moiety is

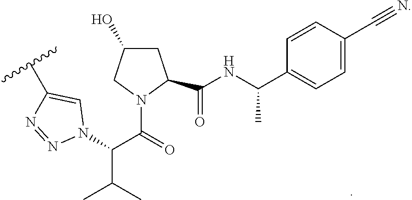 [SMILES: *c1cn([C@H](C(=O)N2C[C@H](O)C[C@H]2C(=O)N[C@@H](C)c2ccc(C#N)cc2)C(C)C)nn1]

In some embodiments, the degradation moiety is

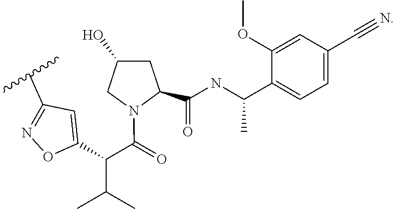 [SMILES: *c1cc([C@H](C(=O)N2C[C@H](O)C[C@H]2C(=O)N[C@@H](C)c2ccc(C#N)cc2OC)C(C)C)on1]

In some embodiments, the degradation moiety is

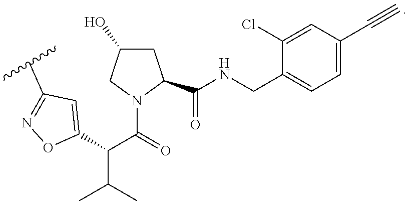 [SMILES: *c1cc([C@H](C(=O)N2C[C@H](O)C[C@H]2C(=O)NCc2ccc(C#C)cc2Cl)C(C)C)on1]

In some embodiments, the degradation moiety is

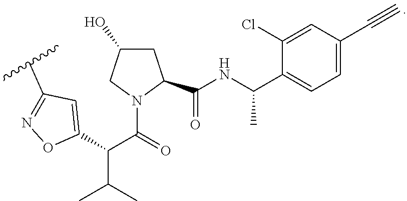 [SMILES: *c1cc([C@H](C(=O)N2C[C@H](O)C[C@H]2C(=O)N[C@@H](C)c2ccc(C#C)cc2Cl)C(C)C)on1]

In some embodiments, the degradation moiety is

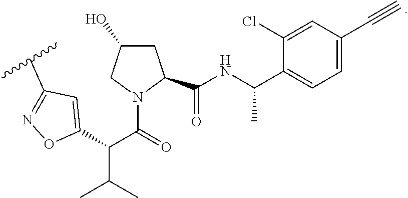 [SMILES: *c1cc([C@H](C(=O)N2C[C@H](O)C[C@H]2C(=O)N[C@@H](C)c2ccc(C#C)cc2Cl)C(C)C)on1]

In some embodiments, the degradation moiety is

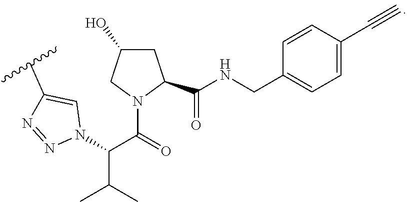 [SMILES: *c1cn([C@H](C(=O)N2C[C@H](O)C[C@H]2C(=O)NCc2ccc(C#C)cc2)C(C)C)nn1]

In some embodiments, the degradation moiety is

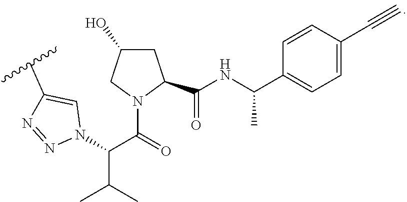 [SMILES: *c1cn([C@H](C(=O)N2C[C@H](O)C[C@H]2C(=O)N[C@@H](C)c2ccc(C#C)cc2)C(C)C)nn1]

In some embodiments the degradation moiety is

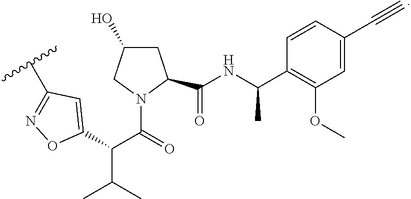 [SMILES: *c1cc([C@H](C(=O)N2C[C@H](O)C[C@H]2C(=O)N[C@H](C)c2ccc(C#C)cc2OC)C(C)C)on1 ; "."]

In some embodiments, the degradation moiety is

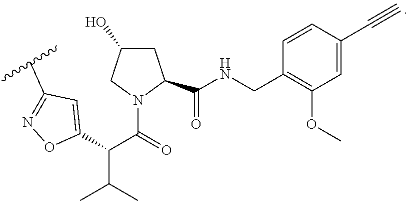 [SMILES: *c1cc([C@H](C(=O)N2C[C@H](O)C[C@H]2C(=O)NCc2ccc(C#C)cc2OC)C(C)C)on1]

In some embodiments, the degradation moiety is

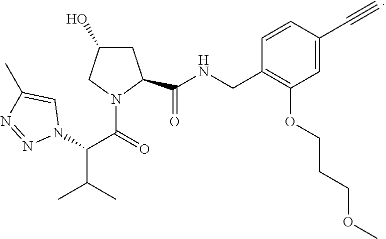 [SMILES: C#Cc1ccc(CNC(=O)[C@@H]2C[C@@H](O)CN2C(=O)[C@H](C(C)C)n2cc(C)nn2)c(OCCCOC)c1 ; "."]

In some embodiments, the degradation moiety, is

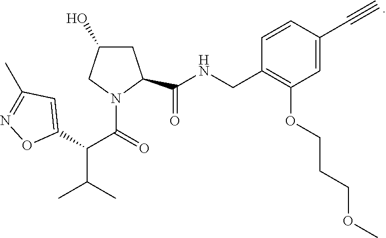 [SMILES: C#Cc1ccc(CNC(=O)[C@@H]2C[C@@H](O)CN2C(=O)[C@@H](c2cc(C)no2)C(C)C)c(OCCCOC)c1 ; "."]

In some embodiments, the degradation moiety is

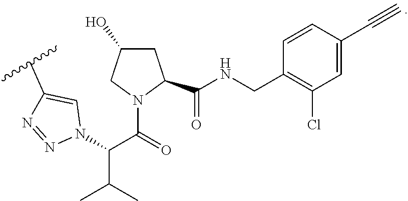 [SMILES: *c1cn([C@H](C(=O)N2C[C@H](O)C[C@H]2C(=O)NCc2ccc(C#C)cc2Cl)C(C)C)nn1]

In some embodiments, the degradation moiety is

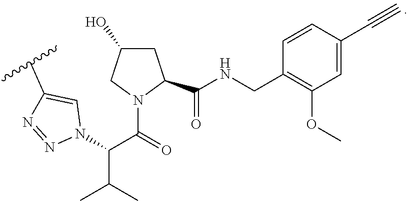 [SMILES: *c1cn([C@H](C(=O)N2C[C@H](O)C[C@H]2C(=O)NCc2ccc(C#C)cc2OC)C(C)C)nn1]

In some embodiments the degradation moiety has the structure of Formula Da:

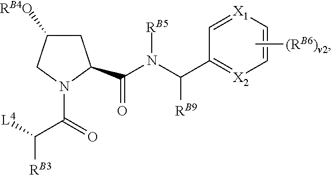

where
L⁴ is —N(R^{B1})(R^{B2}),

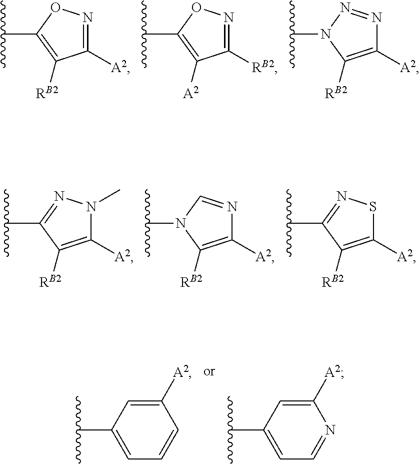

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl,
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁ aryl;
R^{B5} is H, optionally substituted C₁-C₆ alkyl, or optionally substituted C₁-C₆ heteroalkyl;
Each of X₁ and X₂ are, independently, C, N, or O.
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A², or a pharmaceutically acceptable salt thereof.

In some embodiments, the degradation moiety has the structure of Formula Da3.

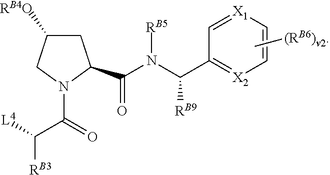

In some embodiments, the degradation moiety has the structure of Formula Da1:

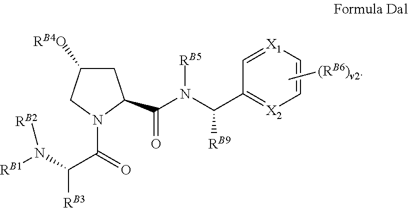

In some embodiments, the degradation moiety has the structure of Formula Da2:

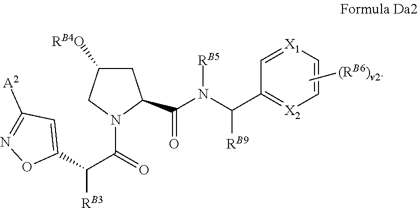

In some embodiments, R^{B9} is optionally substituted C₁-C₆ alkyl. In some embodiments, R^{B9} is methyl.

In some embodiments, R^{B9} is bonded to(S)-stereogenic center.

In some embodiments, v2 is 0. In some embodiments, R^{B4} is H. In some embodiments, R^{B5} is H. In some embodiments, R^{B3} is optionally substituted C₁-C₆ alkyl. In some embodiments, R^{B3} is isopropyl. In some embodiments, R^{B2} is H. In some embodiments, X₁ is C. In some embodiments, X₂ is N.

In some embodiments, the degradation moiety is

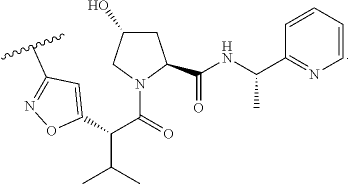 [SMILES: *c1cc([C@H](C(=O)N2C[C@H](O)C[C@H]2C(=O)N[C@@H](C)c2ccccn2)C(C)C)on1]

In some embodiments, the degradation moiety has the structure of Formula E:

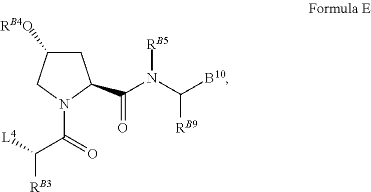

where
L⁴ is —N(R^{B1}) (R^{B2}),

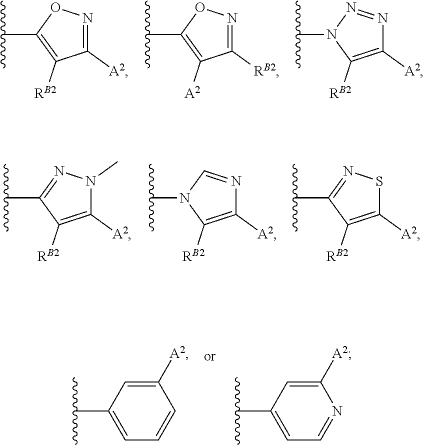

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
R^{B9} is H, optionally substituted C₁-C₆ alkyl, optionally substituted C₃-C₆ alkynyl, optionally substituted C₃-C₁₀ carbocyclyl, or optionally substituted C₂-C₁₀ heterocyclyl;
R^{B10} is, H, optionally substituted C₁-C₆ alkyl, optionally substituted C₃-C₆ alkynyl, optionally substituted C₃-C₁₀ carbocyclyl, optionally substituted C₂-C₁₀ heterocyclyl, optionally substituted amino, or cyano, and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula E3.

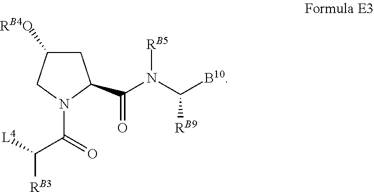

In some embodiments, the degradation moiety has the structure of Formula E1:

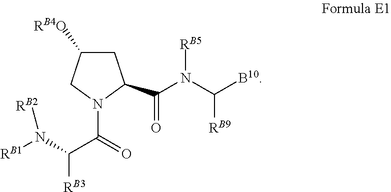

In some embodiments, the degradation moiety is

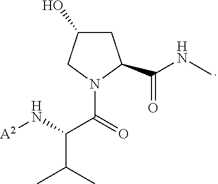

In some embodiments, the degradation moiety is

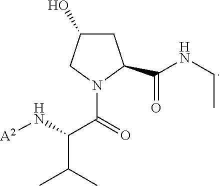

In some embodiments, the degradation moiety has the structure of Formula E2:

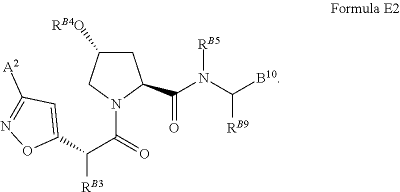

In some embodiments, R^{B9} is optionally substituted C₁-C₆ alkyl. In some embodiments, R^{B9} is methyl.

In some embodiments, R^{B9} is bonded to(S)-stereogenic center.

In some embodiments, v2 is 0. In some embodiments, v2 is 1. In some embodiments, R^{B4} is H. In some embodiments, R^{B3} is H. In some embodiments, R^{B3} is optionally substituted C₁-C₆ alkyl. In some embodiments, R^{B3} is isopropyl. In some embodiments, R^{B2} is H. In some embodiments, R^{B9} is optionally substituted C₁-C₆ alkyl. In some embodiments, R^{B9} is methyl. In some embodiments, R^{B9} is H. In some embodiments, R^{B9} is optionally substituted C₃-C₆ alkynyl. In some embodiments, R^{B10} is absent. In some embodiments, R^{B9} is [1.1.1]pentane. In some embodiments, R^{B9} cyclopropane. In some embodiments, R^{B9} is cyclobutane. In some embodiments, R^{B9} is cyclopentane. In some embodiments, R^{B10} is H. In some embodiments, R^{B10} is cyano. In some embodiments, R^{B10} is optionally substituted C₃-C₁₀ carbocyclyl. In some embodiments, R^{B10} is optionally substituted C₁-C₆ alkyl. In some embodiments, R^{B10} is methyl.

In some embodiments, the degradation moiety is

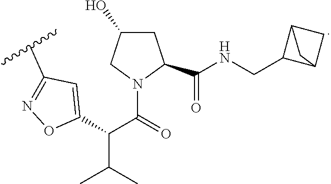 [SMILES: *c1cc([C@H](C(=O)N2C[C@H](O)C[C@H]2C(=O)NCC2C3CC2C3)C(C)C)on1]

In some embodiments, the degradation moiety is

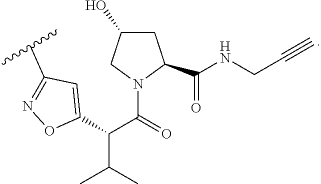 [SMILES: *c1cc([C@H](C(=O)N2C[C@H](O)C[C@H]2C(=O)NCC#C)C(C)C)on1]

In some embodiments, the degradation moiety is

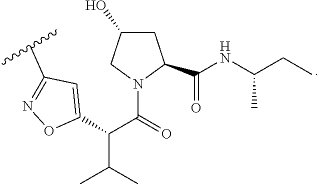 [SMILES: *c1cc([C@H](C(=O)N2C[C@H](O)C[C@H]2C(=O)N[C@@H](C)CC)C(C)C)on1]

In some embodiments, the degradation moiety is

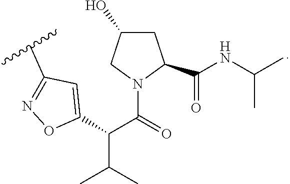 [SMILES: *c1cc([C@H](C(=O)N2C[C@H](O)C[C@H]2C(=O)NC(C)C)C(C)C)on1]

In some embodiments, the degradation moiety is

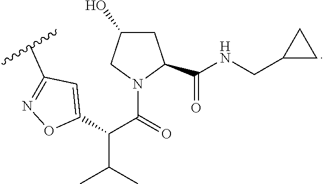 [SMILES: *c1cc([C@H](C(=O)N2C[C@H](O)C[C@H]2C(=O)NCC2CC2)C(C)C)on1]

In some embodiments, the degradation moiety is

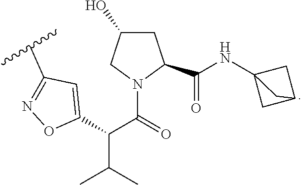 [SMILES: *c1cc([C@H](C(=O)N2C[C@H](O)C[C@H]2C(=O)NC23CC(C2)C3)C(C)C)on1]

In some embodiments, the degradation moiety is

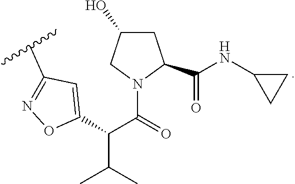 [SMILES: *c1cc([C@H](C(=O)N2C[C@H](O)C[C@H]2C(=O)NC2CC2)C(C)C)on1]

In some embodiments, the degradation moiety is

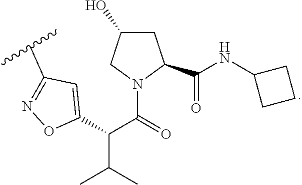 [SMILES: *c1cc([C@H](C(=O)N2C[C@H](O)C[C@H]2C(=O)NC2CCC2)C(C)C)on1]

In some embodiments, the degradation moiety is

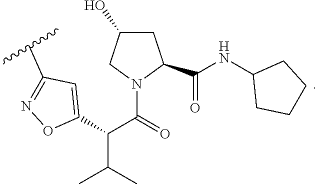 [SMILES: *c1cc([C@H](C(=O)N2C[C@H](O)C[C@H]2C(=O)NC2CCCC2)C(C)C)on1]

In some embodiments, the degradation moiety has the structure of Formula F:

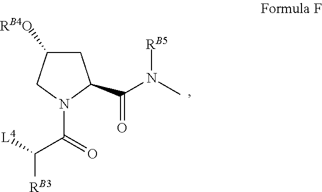

where

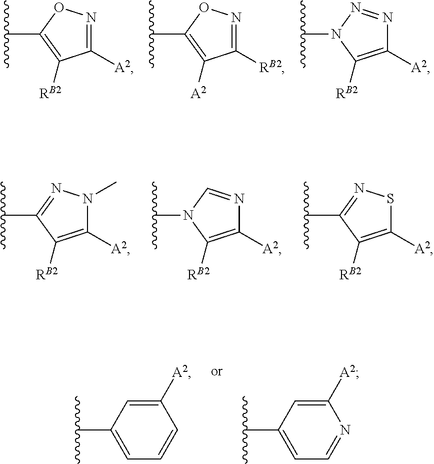

L⁴ is —N(R^{B1})(R^{B2}),
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
A² is a bond between the degradation moiety and the linker;
where one and only one of R^{B1} or R^{B3} is A²,
or a pharmaceutically acceptable salt thereof.

In some embodiments, the degradation moiety has the structure of Formula F3.

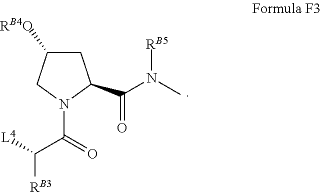

In some embodiments, the degradation moiety has the structure of Formula F1:

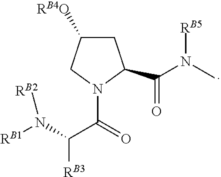

In some embodiments, the degradation moiety is

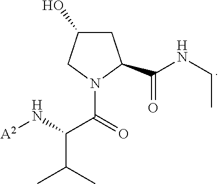

In some embodiments, the degradation moiety is

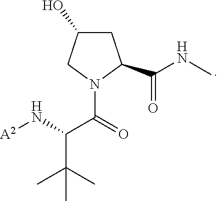

In some embodiments, the degradation moiety is

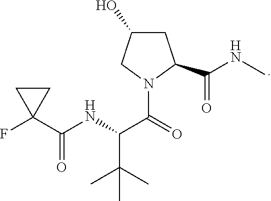 [SMILES: CNC(=O)[C@@H]1C[C@@H](O)CN1C(=O)[C@@H](NC(=O)C1(F)CC1)C(C)(C)C]

In some embodiments, the degradation moiety has the structure of Formula F2:

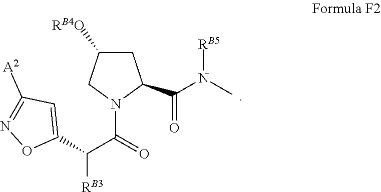

In some embodiments, R^{B9} is optionally substituted C₁-C₆ alkyl. In some embodiments, R^{B9} is methyl.

In some embodiments, R^{B4} is H. In some embodiments, RBS is H. In some embodiments, R^{B3} is optionally substituted C₁-C₆ alkyl. In some embodiments, R^{B3} is isopropyl. In some embodiments, R^{B2} is H.

In some embodiments, the degradation moiety is

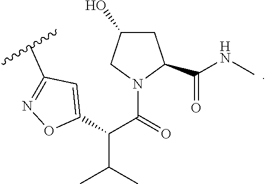 [SMILES: *c1cc([C@H](C(=O)N2C[C@H](O)C[C@H]2C(=O)NC)C(C)C)on1]

In some embodiments, the linker has the structure of Formula II:

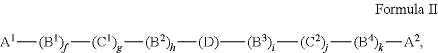

or a pharmaceutically acceptable salt thereof,
where
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₃-C₁₀ carbocyclyl, optionally substituted C₂-C₈ heterocyclyl, optionally substituted C₂-C₆ heteroaryl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, optionally substituted C₃-C₁₀ cycloalkyl, optionally substituted C₃-C₁₀ carbocyclyl, or optionally substituted C₁₋₁₀ heteroalkyl; or D is absent, and the linker is A¹-(B¹)_{f}—(C¹)_{g}—(B²)ₕ—(B³)ᵢ—(C²)ⱼ—(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₁₀ heterocyclyl, optionally substituted C₂₋₆ heteroaryl, O, or NR^{N}; and D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₁₀ heterocyclyl, optionally substituted C₂₋₆ heteroaryl, optionally substituted C₃-C₁₀ cycloalkyl, optionally substituted C₃-C₁₀ carbocyclyl, O, or NR^{N}.

In some embodiments, each of B¹ and B⁴ is, independently,

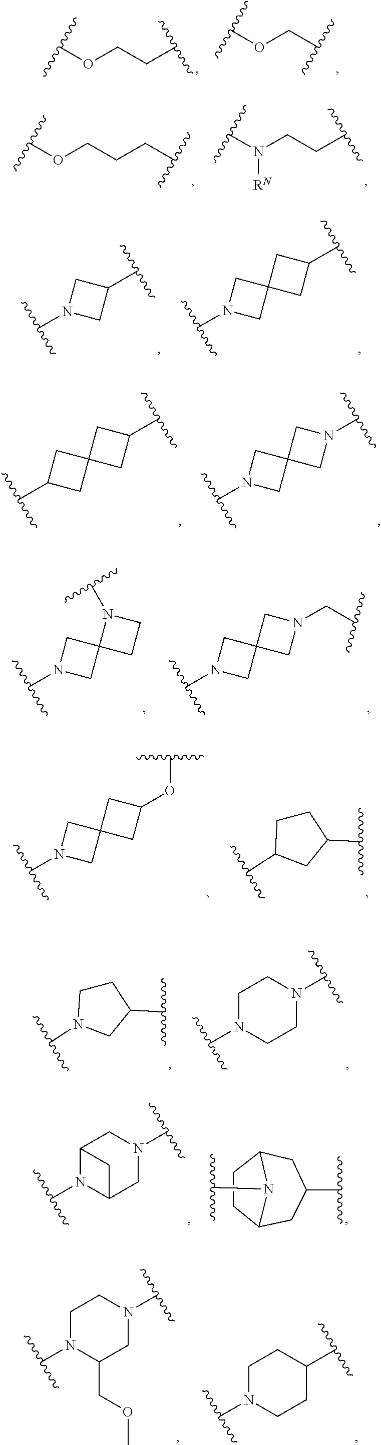

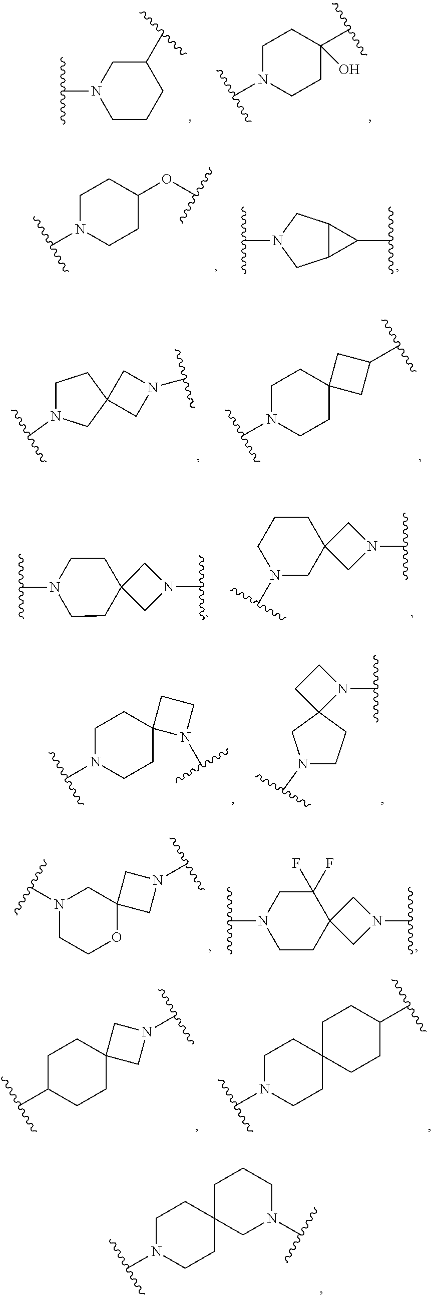

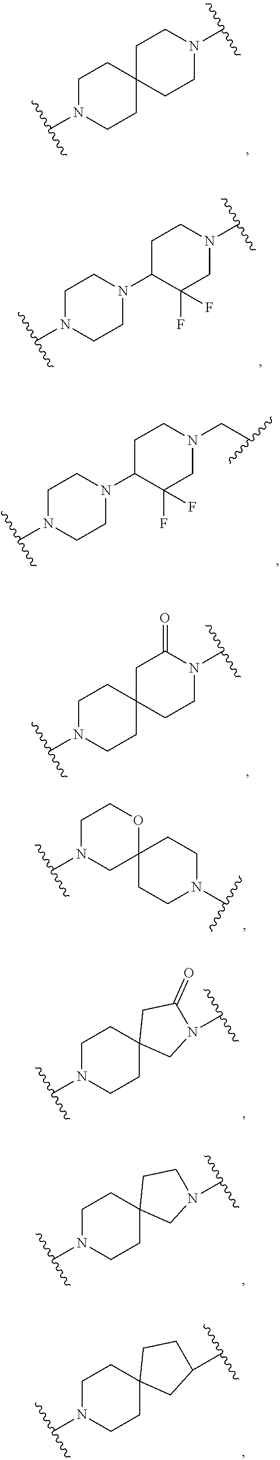

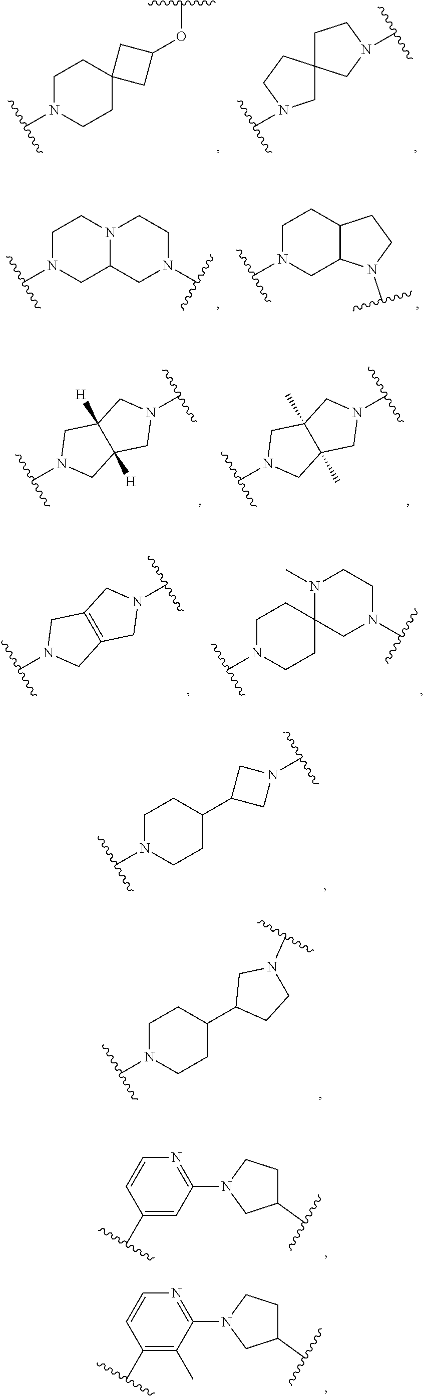

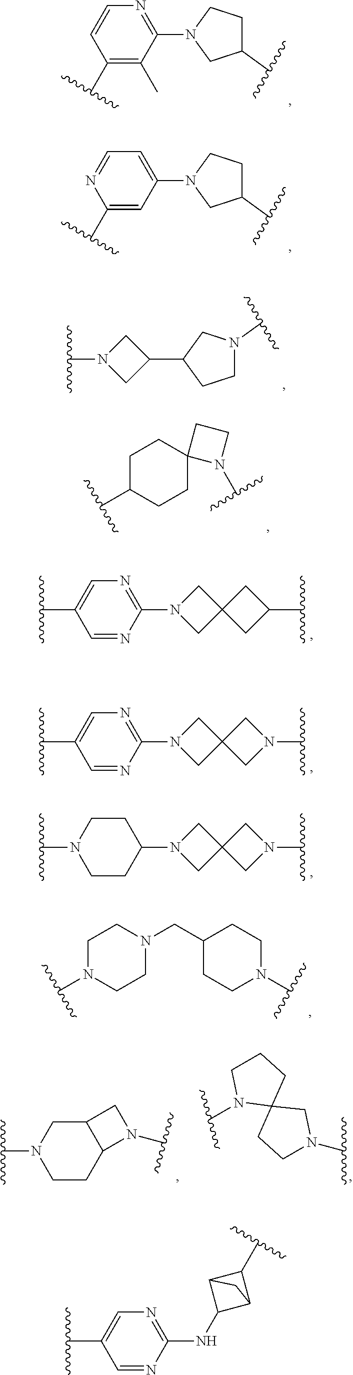

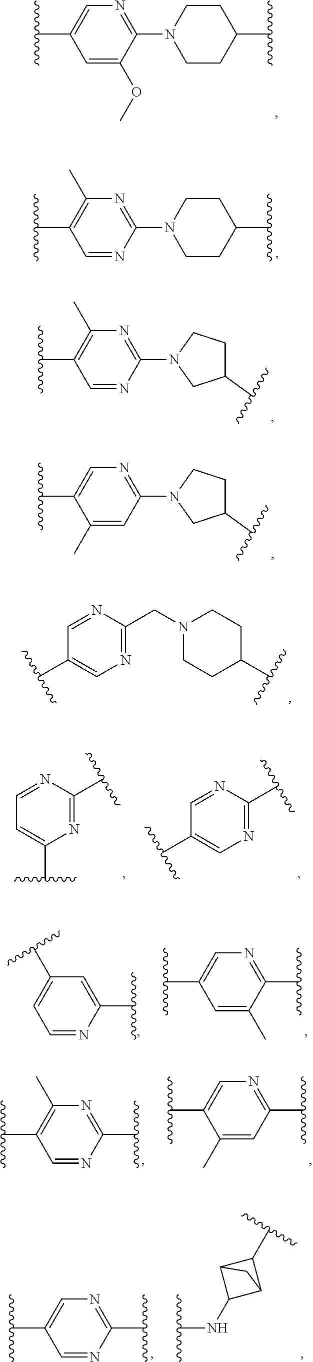

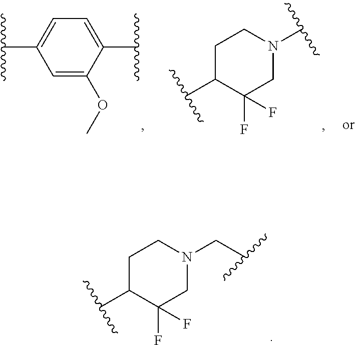

In some embodiments, B¹ is

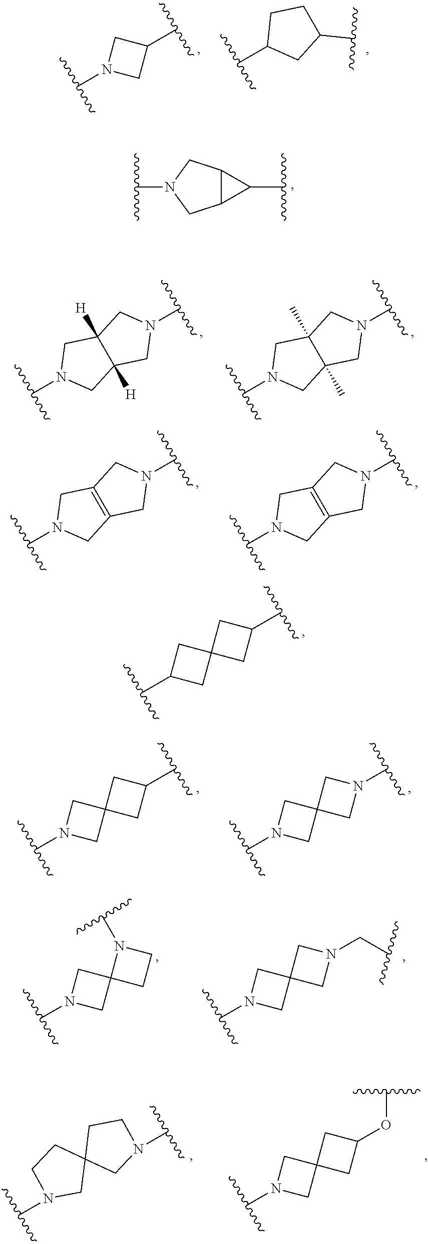

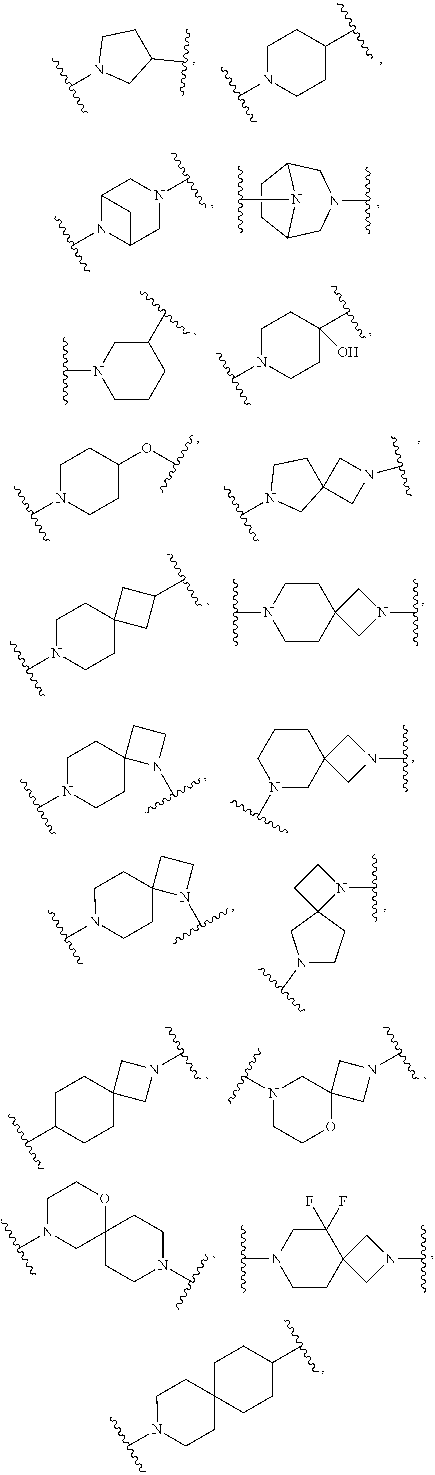

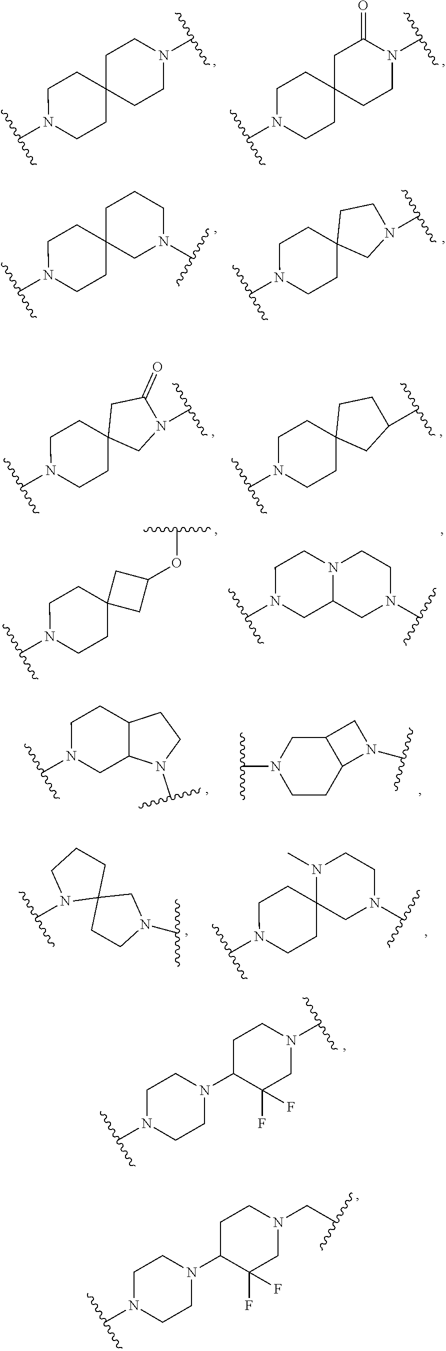

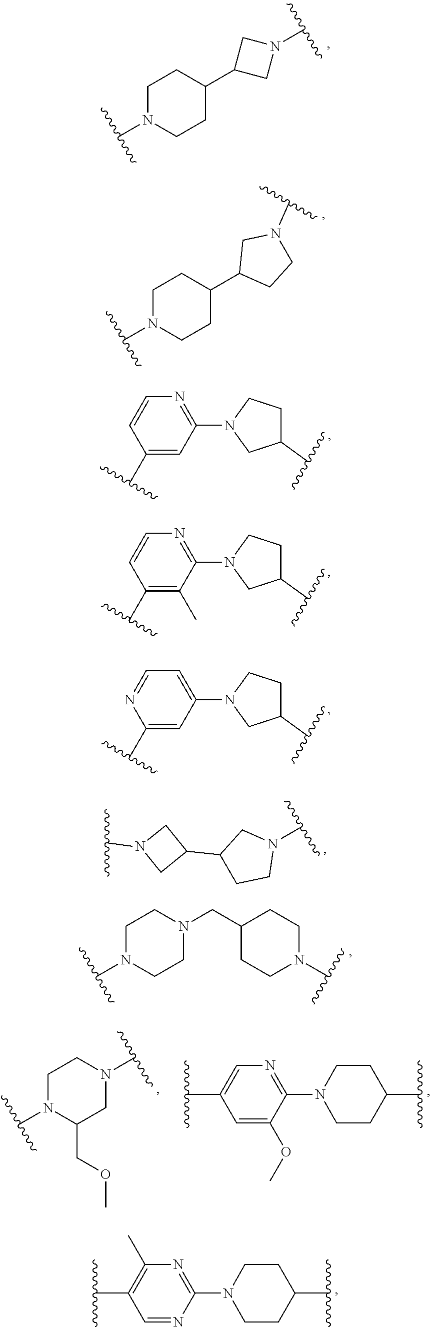

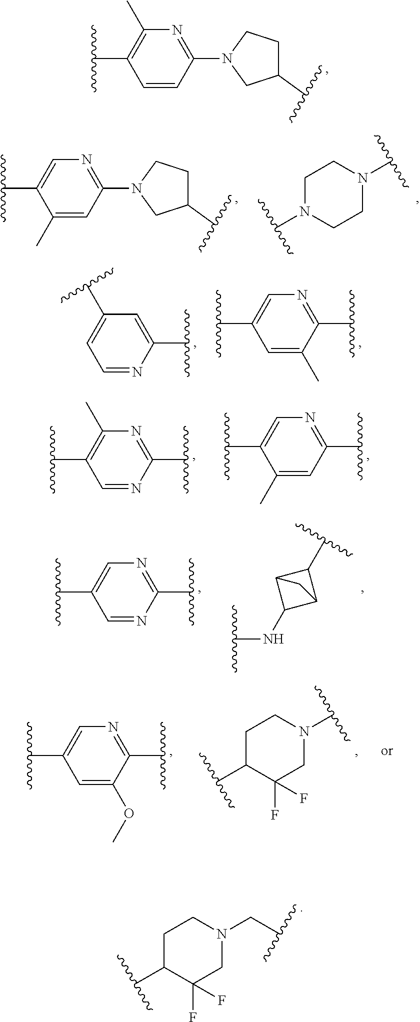

In some embodiments, B⁴ is

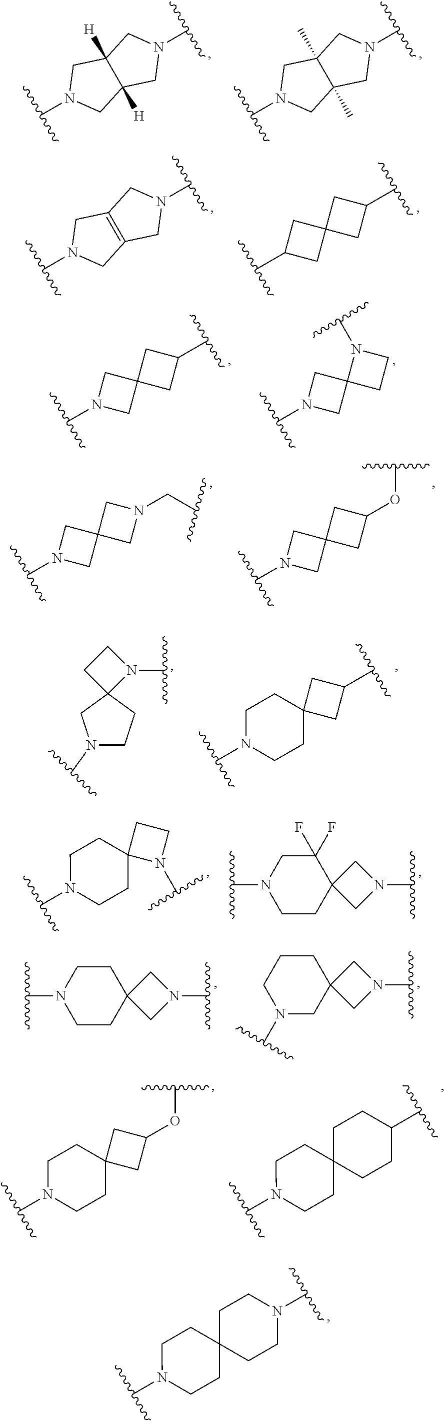

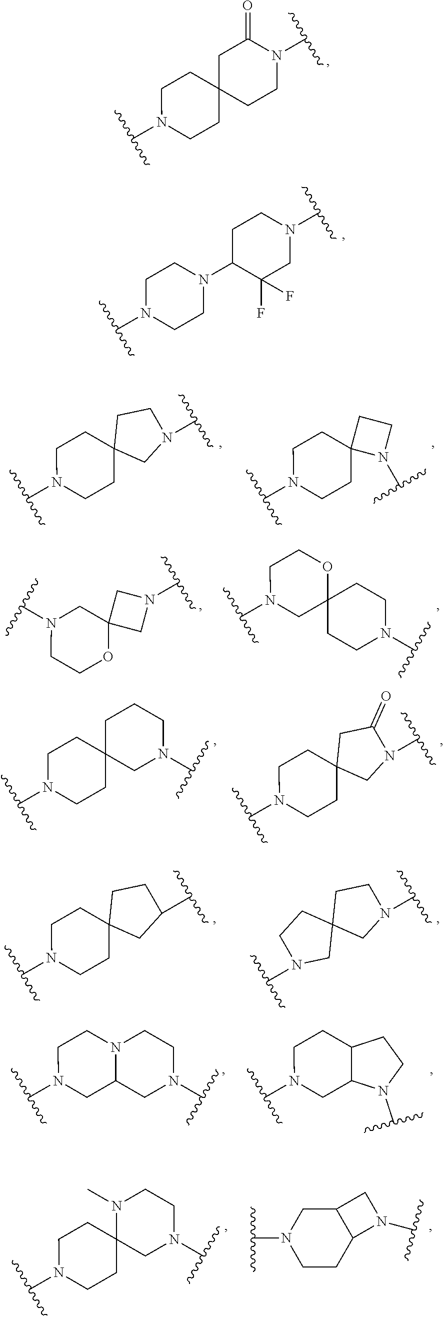

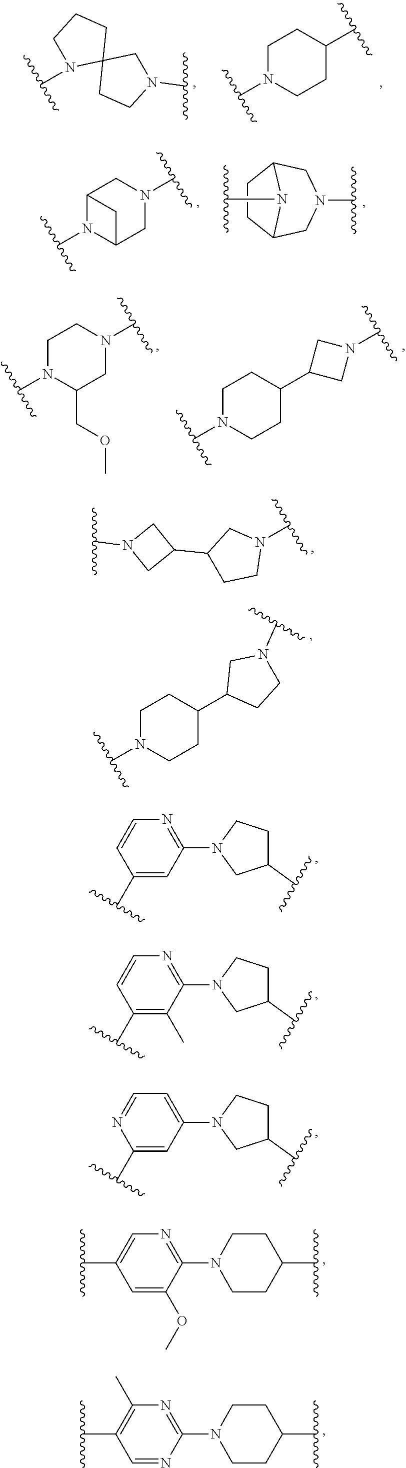

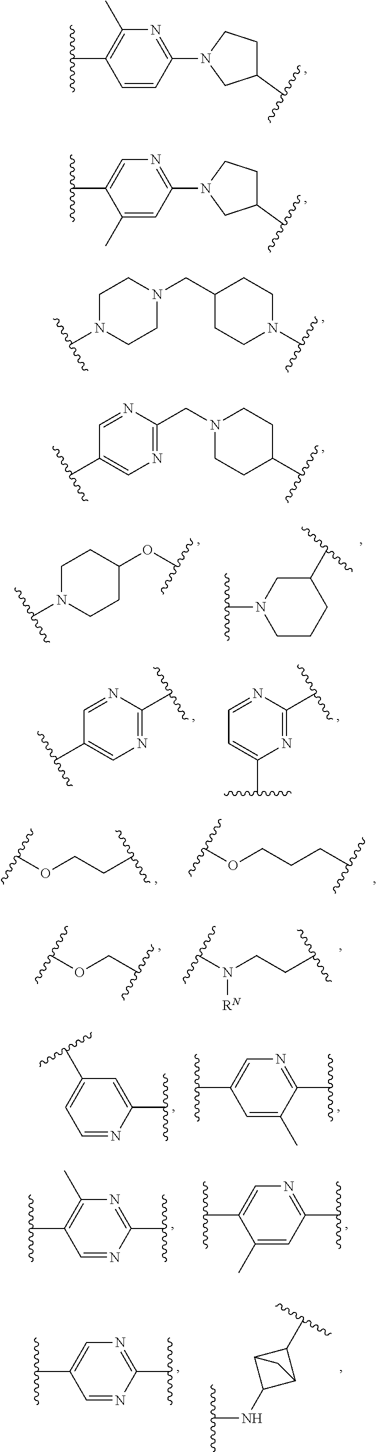

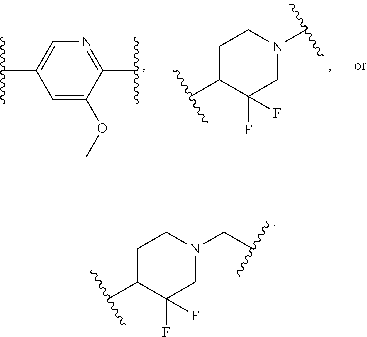

In some embodiments, C¹ is

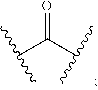 [SMILES: *C(*)=O]

In some embodiments, B² is optionally substituted C₁-C₄ alkyl.

In some embodiments, D is optionally substituted C₁-C₁₀ alkyl.

In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, D is absent, and the linker is A¹-(B¹)_{f}—(C¹)_{g}—(B²)ₕ—(B³)ᵢ—(C²)ⱼ—(B⁴)ₖ-A².

In some embodiments, the linker is D. In some embodiments, D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl. In some embodiments, D is optionally substituted C₃-C₁₀ cycloalkyl, f is 1, g is 0, h is 0, i is 0, j is 0, and, k is 1. In some embodiments, D is optionally substituted C₅-C₁₀ cycloalkyl, f is 1, g is 0, h is 0, i is 0, j is 0, and, k is 0. In some embodiments, D is optionally substituted C₃-C₁₀ cycloalkyl, f is 0, g is 0, h is 0, i is 0, j is 0, and, k is 1. In some embodiments, D is optionally substituted C₃-C₁₀ cycloalkyl, f is 0, g is 0, h is 0, i is 0, j is 0, and, k is 0. In some embodiments, D is optionally substituted C₃-C₁₀ carbocyclyl, f is 1, g is 0, h is 0, i is 0, j is 0, and, k is 1. In some embodiments, D is optionally substituted C₃-C₁₀ carbocyclyl, f is 1, g is 0, h is 0, i is 0, j is 0, and, k is 0. In some embodiments, D is optionally substituted C₃-C₁₀ carbocyclyl, f is 0, g is 0, h is 0, i is 0, j is 0, and, k is 1. In some embodiments, D is optionally substituted C₃-C₁₀ carbocyclyl, f is 0, g is 0, h is 0, i is 0, j is 0, and, k is 0. In some embodiments, D is:

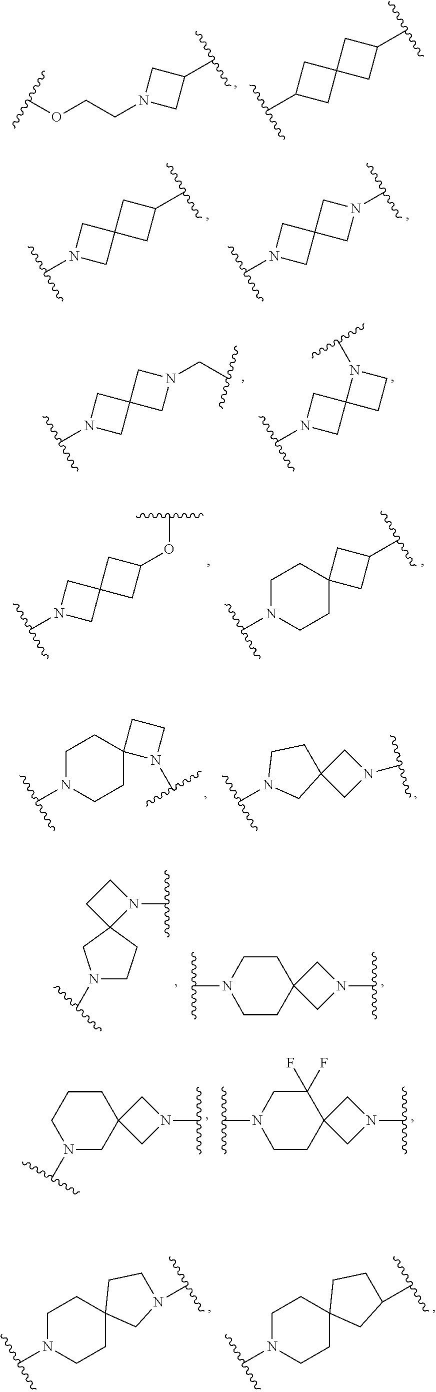

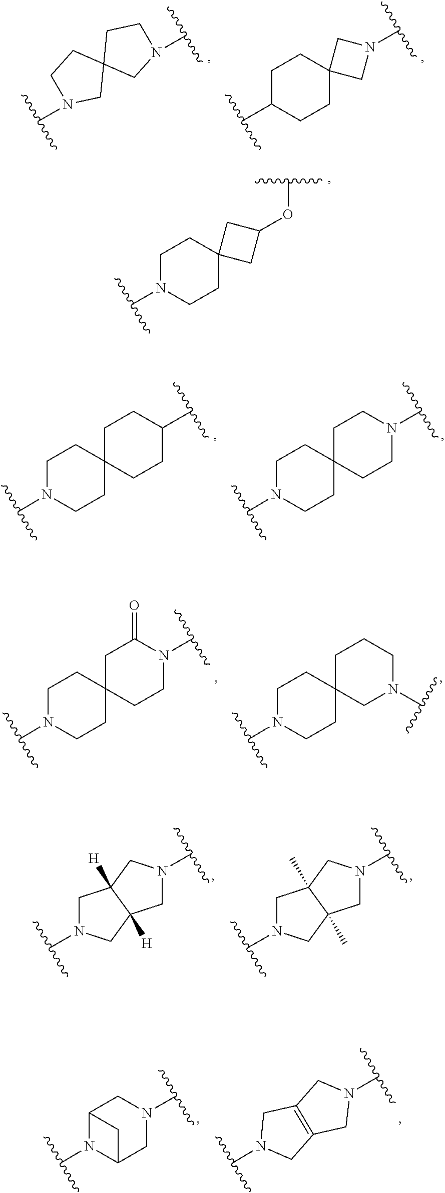

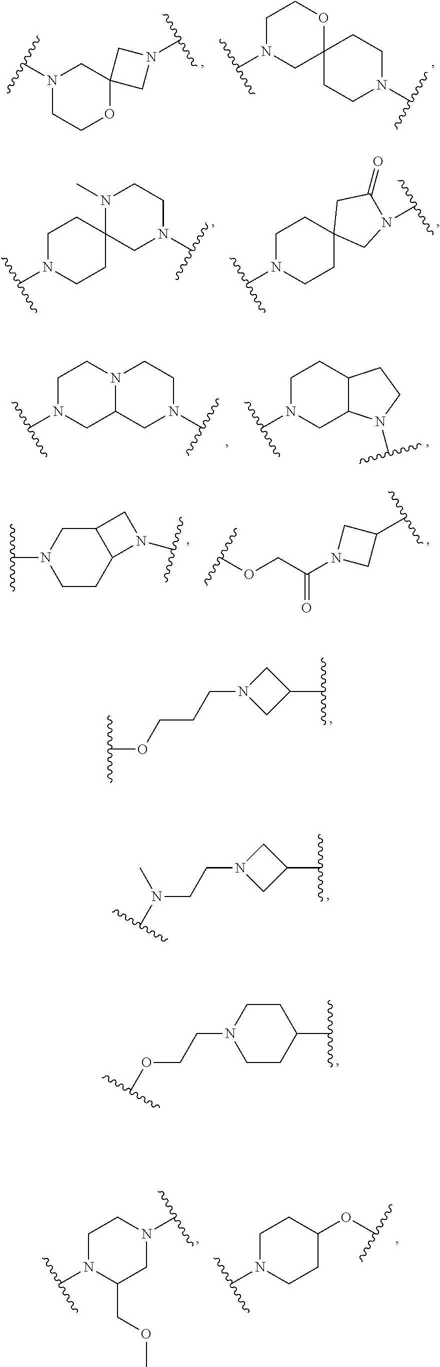

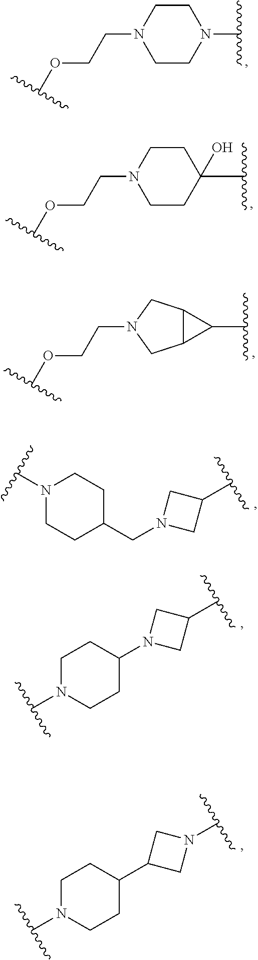 [SMILES: *OCCN1CCN(*)CC1 ; *OCCN1CCC(*)(O)CC1 ; *OCCN1CC2C(*)C2C1 ; *C1CN(CC2CCN(*)CC2)C1 ; *C1CN(C2CCN(*)CC2)C1 ; *N1CCC(C2CN(*)C2)CC1]

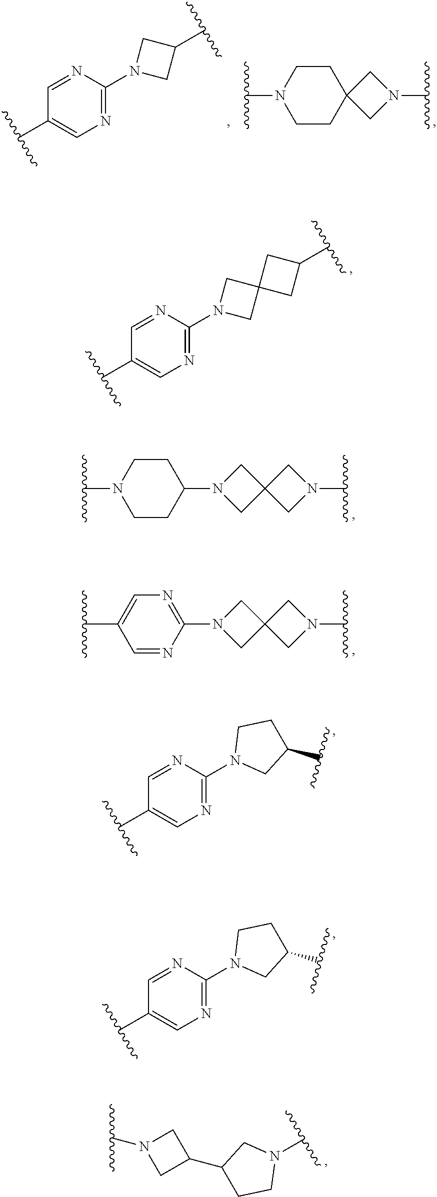

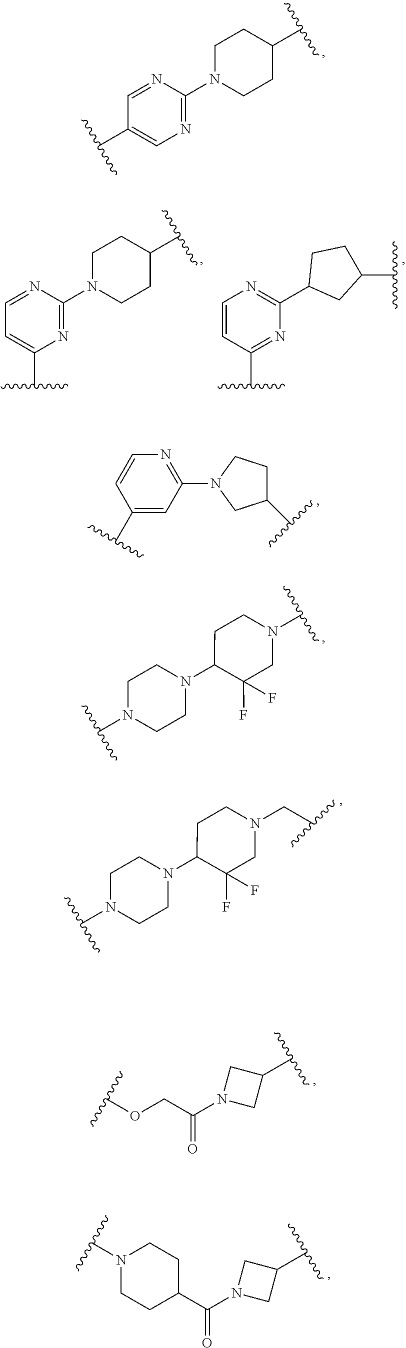

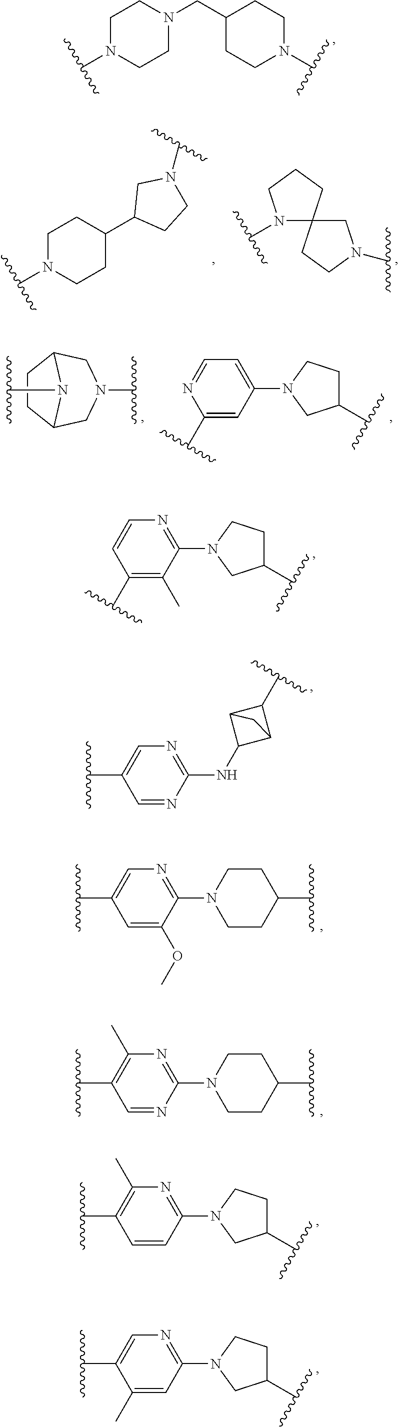

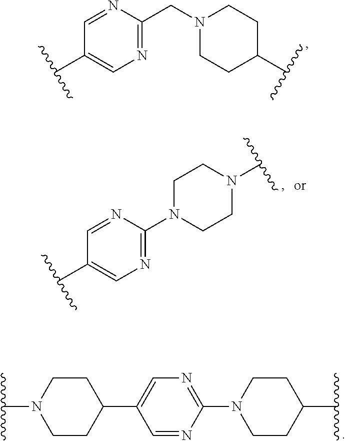 [SMILES: *c1cnc(CN2CCC(*)CC2)nc1 ; *c1cnc(N2CCN(*)CC2)nc1 ; *C1CCN(c2ncc(C3CCN(*)CC3)cn2)CC1]

In some embodiments, the linker has the structure of

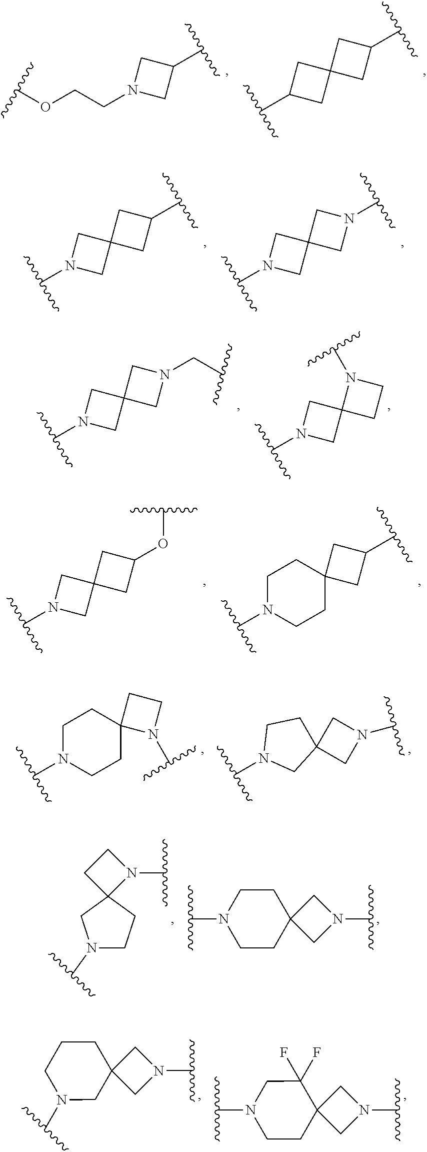

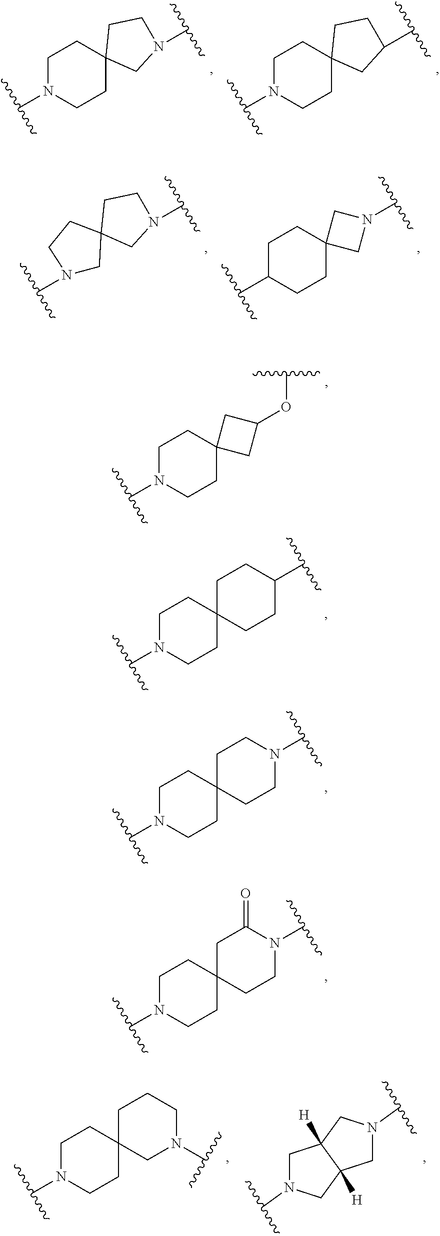

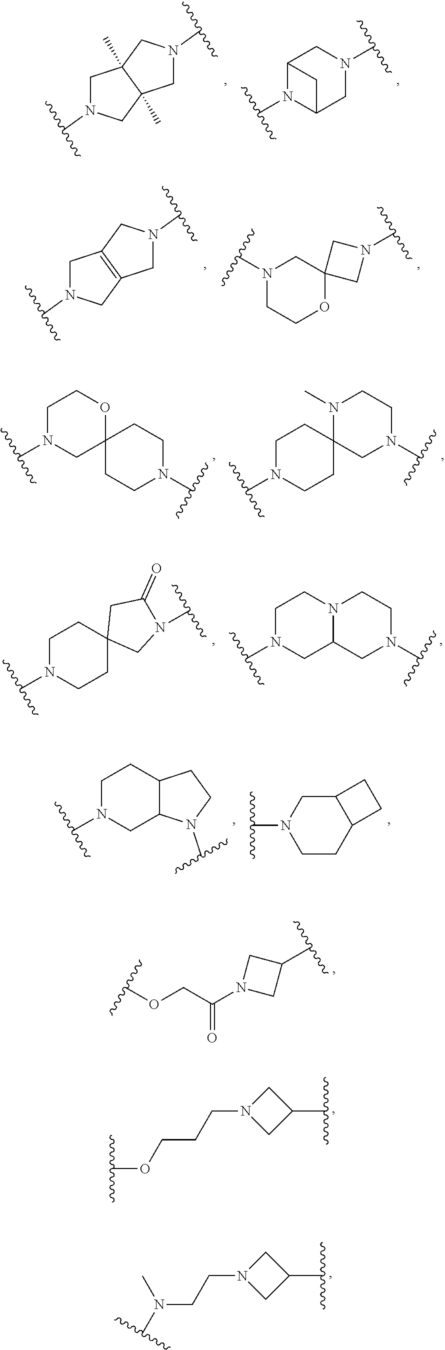

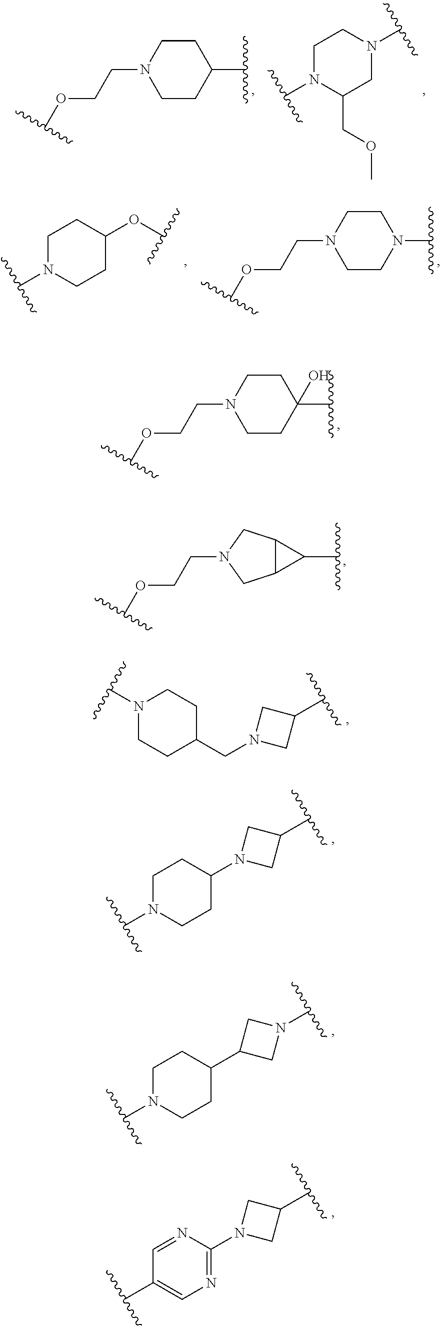

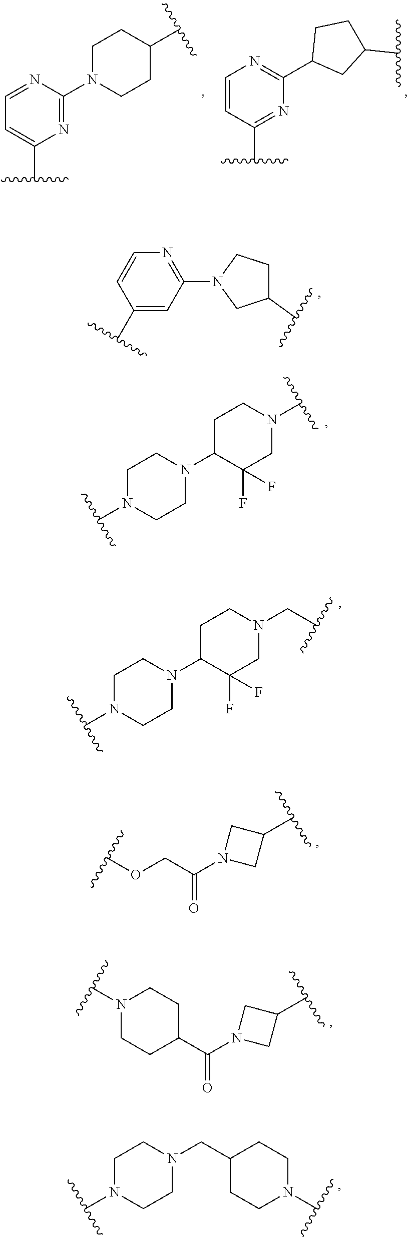

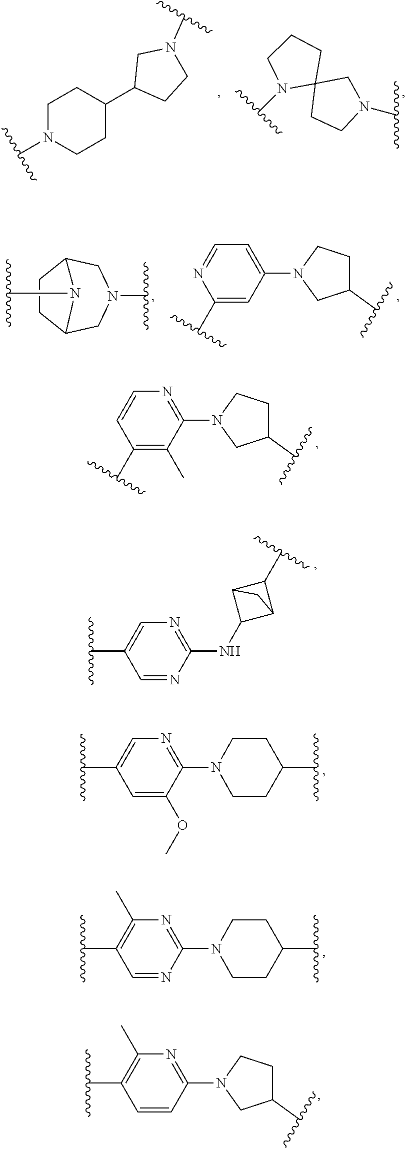

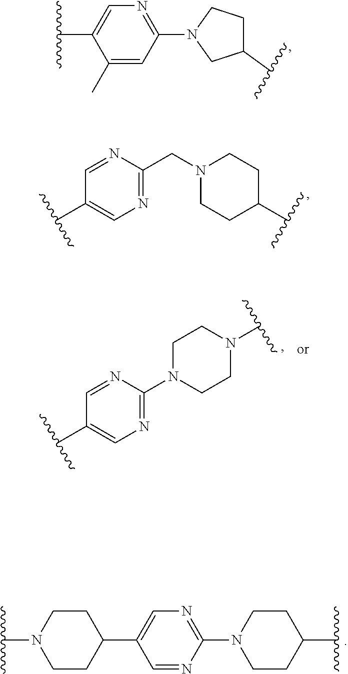 [SMILES: *c1cnc(N2CCC(*)C2)cc1C ; *c1cnc(CN2CCC(*)CC2)nc1 ; *c1cnc(N2CCN(*)CC2)nc1 ; *C1CCN(c2ncc(C3CCN(*)CC3)cn2)CC1]

In some embodiments, the linker has the structure of Formula III:

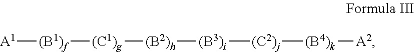

wherein
   A¹ is a bond between the linker and ring system A;
   A² is a bond between the degradation moiety and the linker;
   each of B¹, B², B³, and B⁴ is, independently, optionally substituted ethynyl, optionally substituted C₆-C₁₀ aryl, optionally substituted C₃-C₁₀ cycloalkyl, optionally substituted C₃-C₁₀ carbocyclyl, optionally substituted C₂-C₁₀ heterocyclyl, optionally substituted C₂-C₉ heteroaryl, O, S, S(O)₂, or NR^{N},
   each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₆₋₁₂ aryl, or optionally substituted C₁₋₇ heteroalkyl;
   each of C¹ and C² is, independently, carbonyl, thiocarbonyl, sulphonyl, or phosphoryl; and
   each of f, g, h, i, j, and k is, independently, 0 or 1.
B is a degradation moiety;
each R¹ is independently halo, optionally substituted C₁-C₆ alkyl, optionally substituted C₁-C₆ heteroalkyl, optionally substituted C₃-C₈ cycloalkyl, or optionally substituted C₂-C₁₀ heterocyclyl;
and
each X is, independently, halo.

In some embodiments, the linker is of structure -(L¹)ₙ-, wherein n is 1, 2, or 3, and each L¹ is independently O, NR^{N}, ethynyl, optionally substituted C₂-C₁₀ heterocyclyl, optionally substituted C₂-C₉ heteroaryl, optionally substituted C₆-C₁₀ aryl, or optionally substituted C₃-C₁₀ cycloalkyl.

In some embodiments, at least one L¹ is optionally substituted C₂-C₁₀ heterocyclyl. In some embodiments the optionally substituted C₂-C₁₀ heterocyclyl is 4-, 5-, or 6-membered monocyclic heterocyclyl. In some embodiments the 4-, 5-, or 6-membered monocyclic heterocyclyl is:

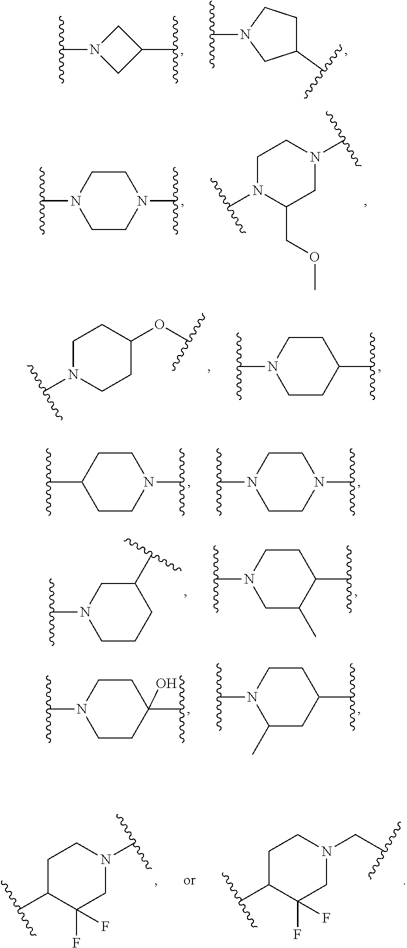

In some embodiments, the optionally substituted C₂-C₁₀ heterocyclyl is a spirocyclic heterocyclyl. In some embodiments, the spirocyclic heterocyclyl is:

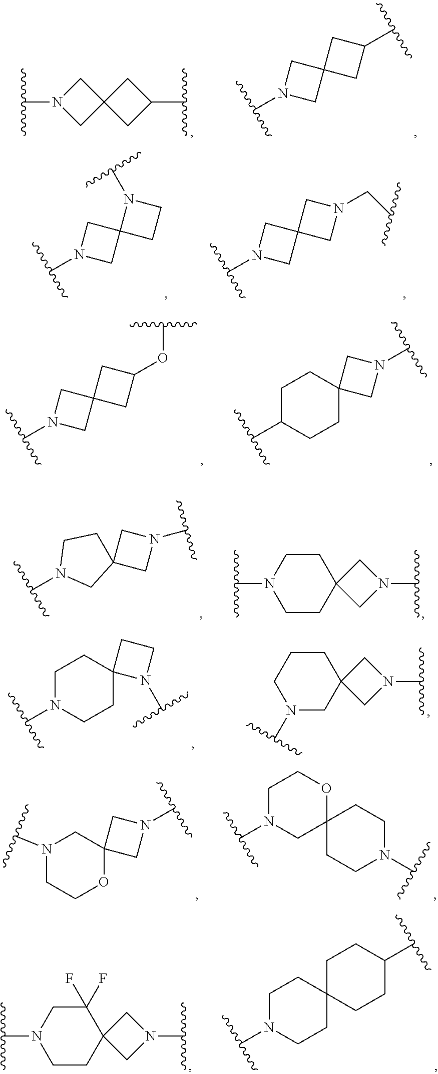

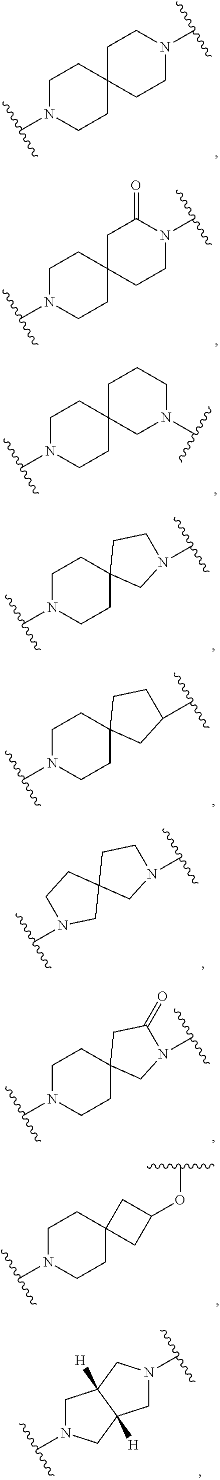

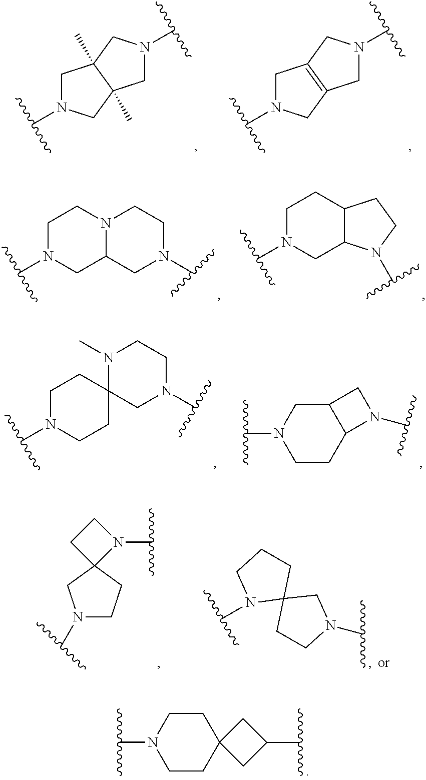

In some embodiments, the optionally substituted C₂-C₁₀ heterocyclyl is a bridged heterocyclyl. In some embodiments the bridged heterocyclyl is:

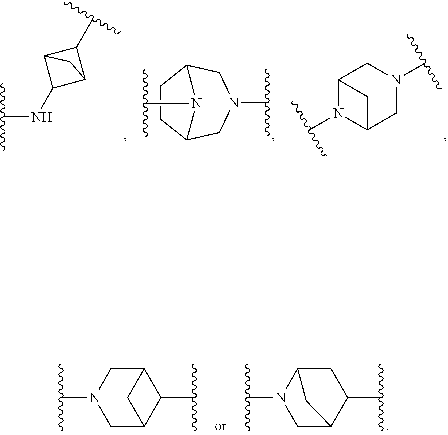 [SMILES: *NC1C2CC1C2* ; *N1CC2CCC(C1)N2* ; *N1CC2CC(C1)N2* ; *C1C2CC1CN(*)C2 ; *C1CC2CC1CN2*]

In some embodiments, the optionally C₂-C₁₀ heterocyclyl is a fused bicyclic heterocyclyl. In some embodiments, the fused bicyclic heterocyclyl is:

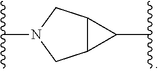 [SMILES: *C1C2CN(*)CC12]

In some embodiments, at least one L¹ is optionally substituted C₂-C₉ heteroaryl. In some embodiments, the linker is -(L¹)_{q}-(optionally substituted C₂-C₉ heteroaryl)-(L¹)_{q}-, wherein each q is independently 0 or 1. In some embodiments, the optionally substituted C₂-C₉ heteroaryl is a 6-membered monocyclic heteroaryl. In some embodiments, the 6-membered monocyclic heteroaryl is:

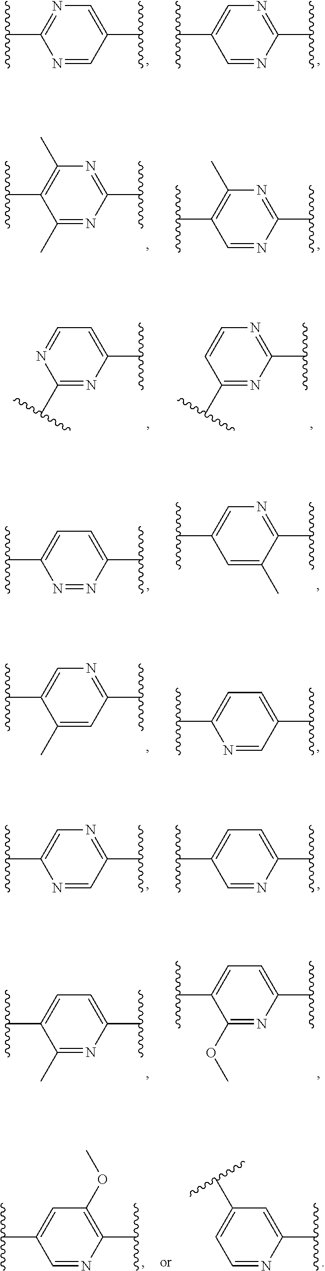

In some embodiments, at least one L¹ is optionally substituted C₂-C₉ heteroaryl. In some embodiments, the linker is:

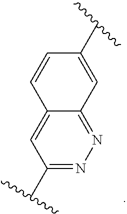 [SMILES: *c1ccc2cc(*)nnc2c1]

In some embodiments, at least one L¹ is optionally substituted C₆-C₁₀ aryl. In some embodiments, the optionally substituted C₆-C₁₀ aryl is a 6-membered monocyclic aryl. In some embodiments, the 6-membered monocyclic aryl is optionally substituted phenyl.

In some embodiments, at least one L¹ is optionally substituted C₃-C₁₀ cycloalkyl. In some embodiments, the optionally substituted C₃-C₁₀ cycloalkyl is a monocyclic cycloalkyl. In some embodiments, the 6-membered monocyclic cycloalkyl is:

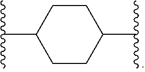 [SMILES: *C1CCC(*)CC1]

In some embodiments, the optionally substituted C₃-C₁₀ cycloalkyl is a bridged cycloalkyl. In some embodiments, the bridged cycloalkyl is:

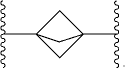 [SMILES: *C12CC(*)(C1)C2]

In some embodiments, at least one L¹ is ethynyl.

In some embodiments, one and only one L¹ is O. In some embodiments, one and only one L¹ is NR^{N}. In some embodiments, R^{N} is optionally substituted C₁-C₄ alkyl. In some embodiments, R^{N} is H.

In some embodiments, the linker is of the following structure:

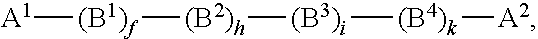

wherein each of B¹, B², B³, and B⁴ is, independently, optionally substituted ethynyl, optionally substituted C₆-C₁₀ aryl, optionally substituted C₃-C₁₀ cycloalkyl, optionally substituted C₂-C₁₀ heterocyclyl, optionally substituted C₂-C₉ heteroaryl, O, or NR^{N}.

In some embodiments, at least one of f, h, i, and k is 1.

In some embodiments, each of B¹, B², B³, and B⁴ is, independently, O, ethynyl, optionally substituted C₂-C₉ heteroaryl, optionally substituted C₂-C₁₀ heterocyclyl, optionally substituted C₃-C₁₀ cycloalkyl, or optionally substituted C₆-C₁₀ aryl. In some embodiments, each of B¹, B², B³, and B⁴ is, independently optionally substituted C₂-C₉ heteroaryl or optionally substituted C₂-C₁₀ heterocyclyl. In some embodiments, each of B¹ and B⁴ is, independently,

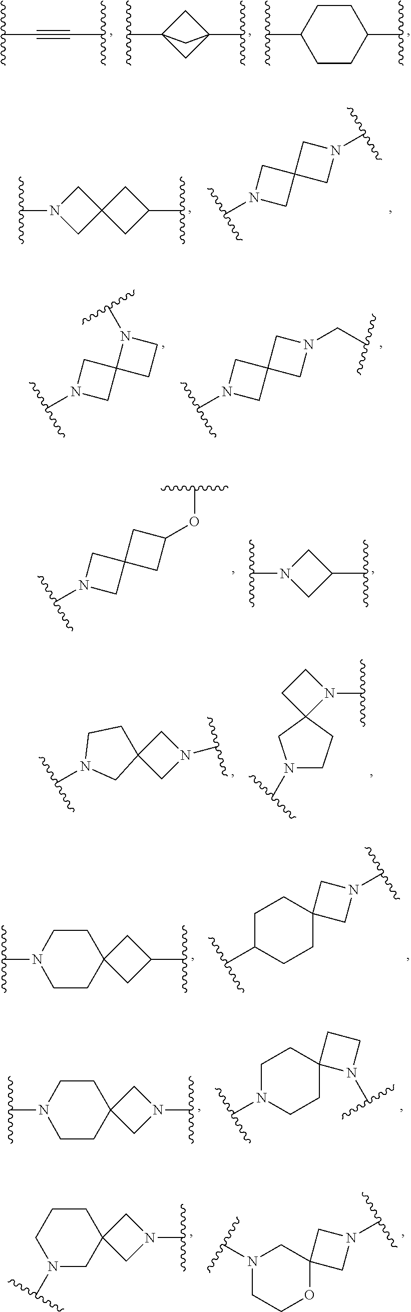

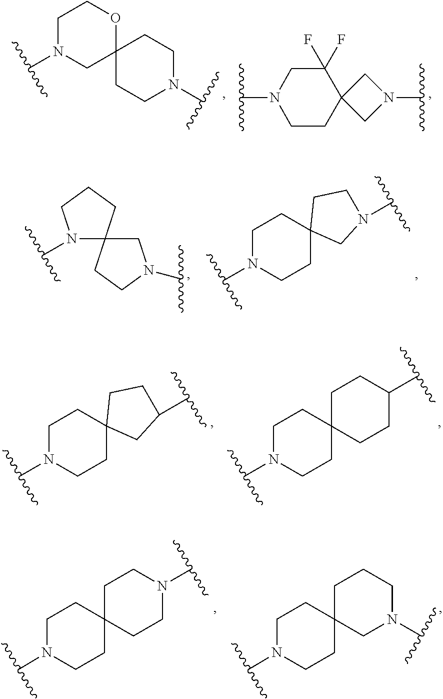

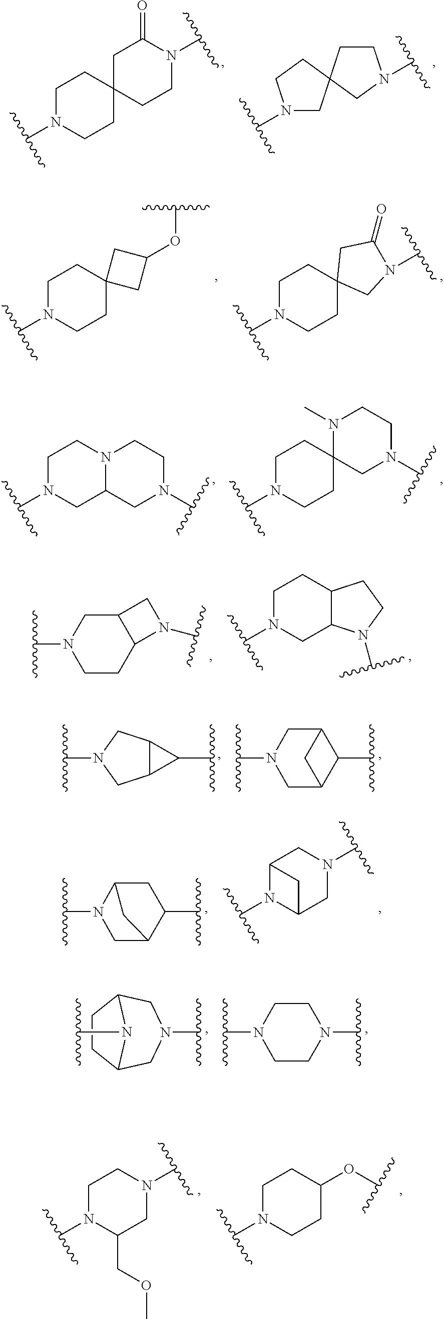

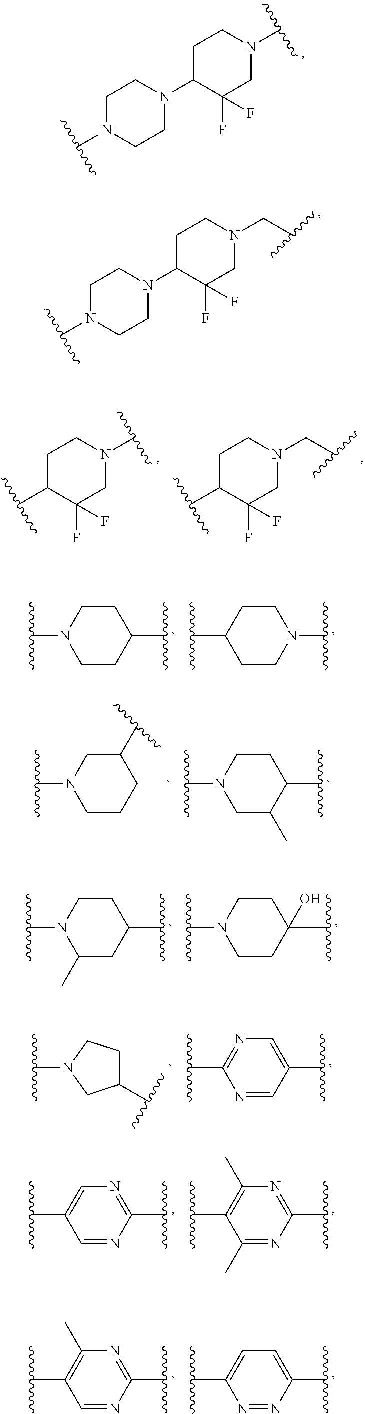

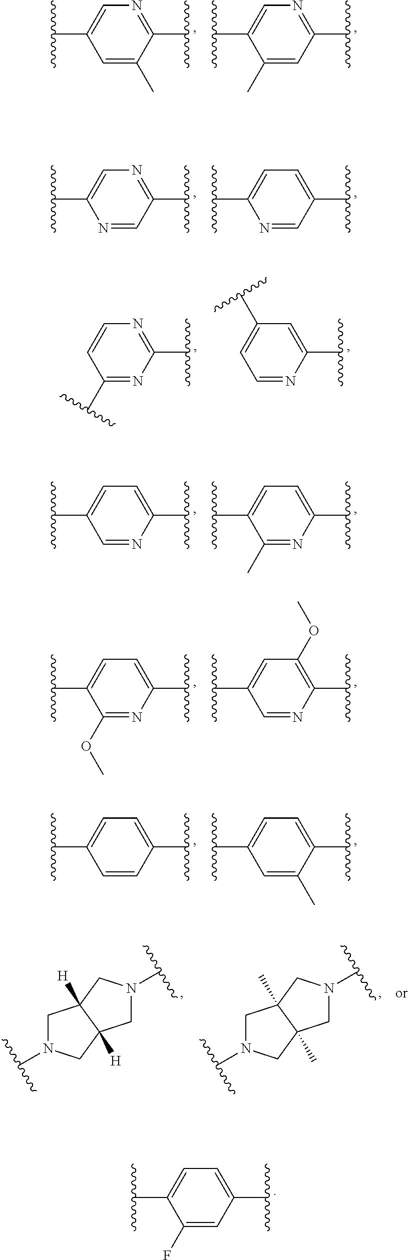

In some embodiments, B¹ is:

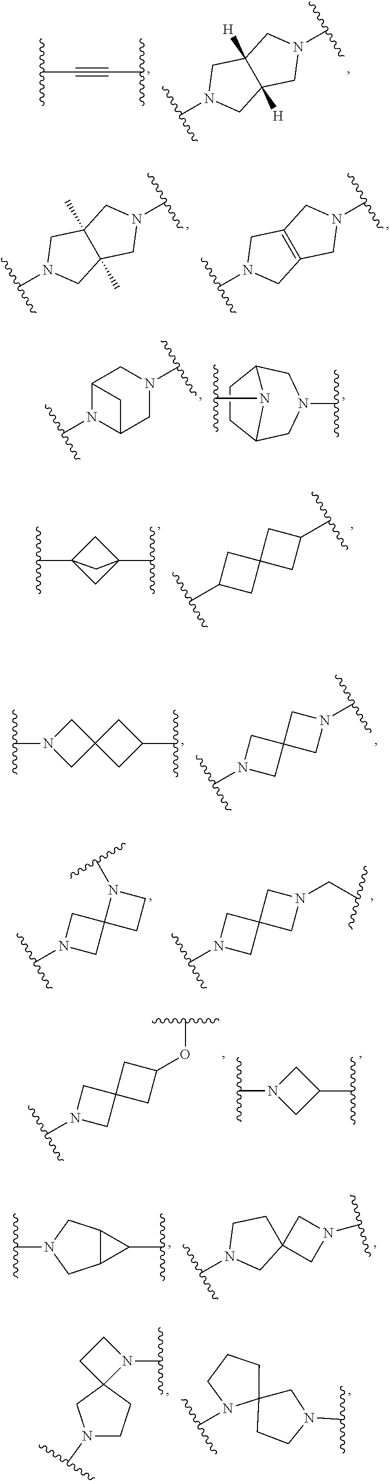

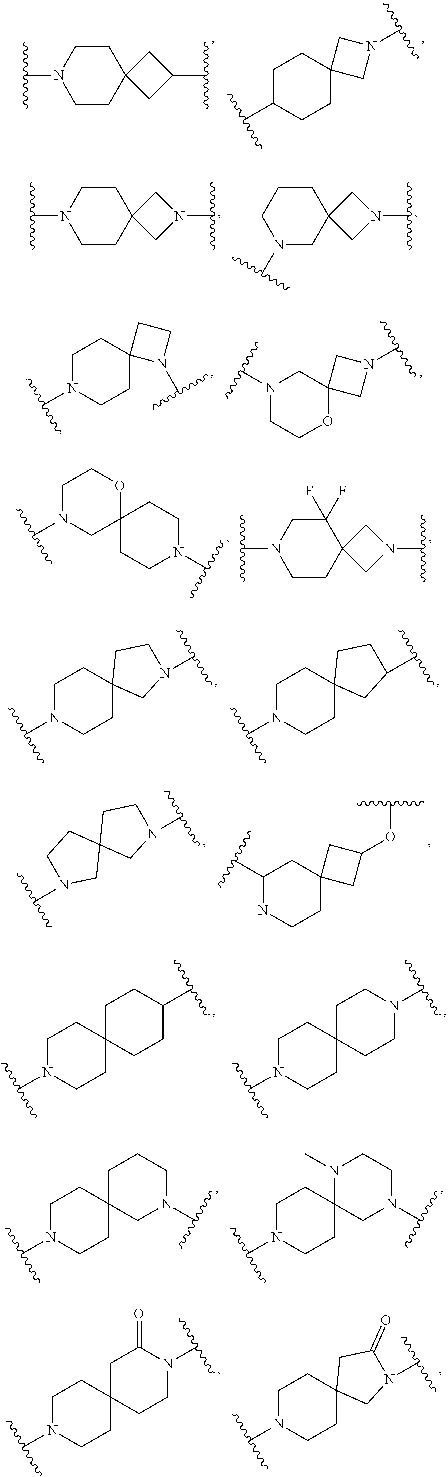

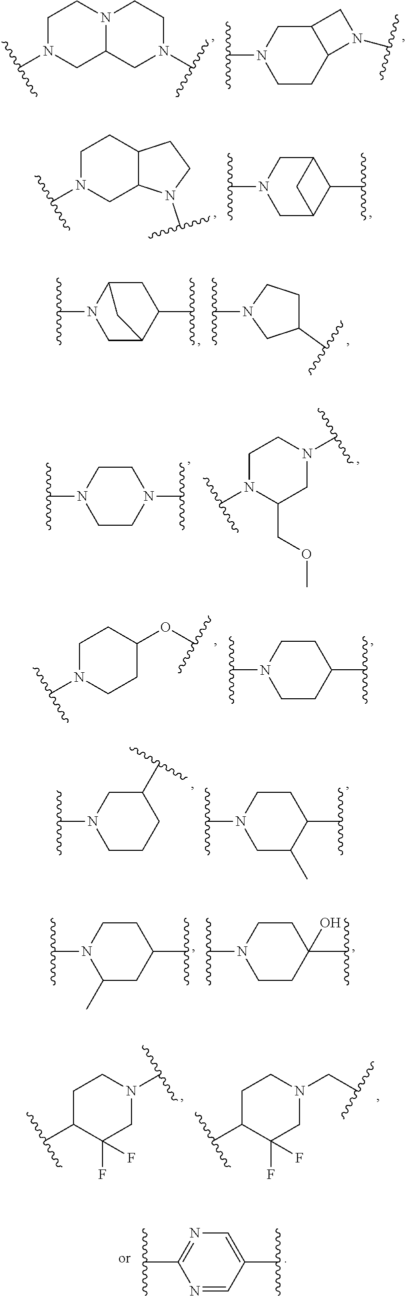

In some embodiments, B⁴ is:

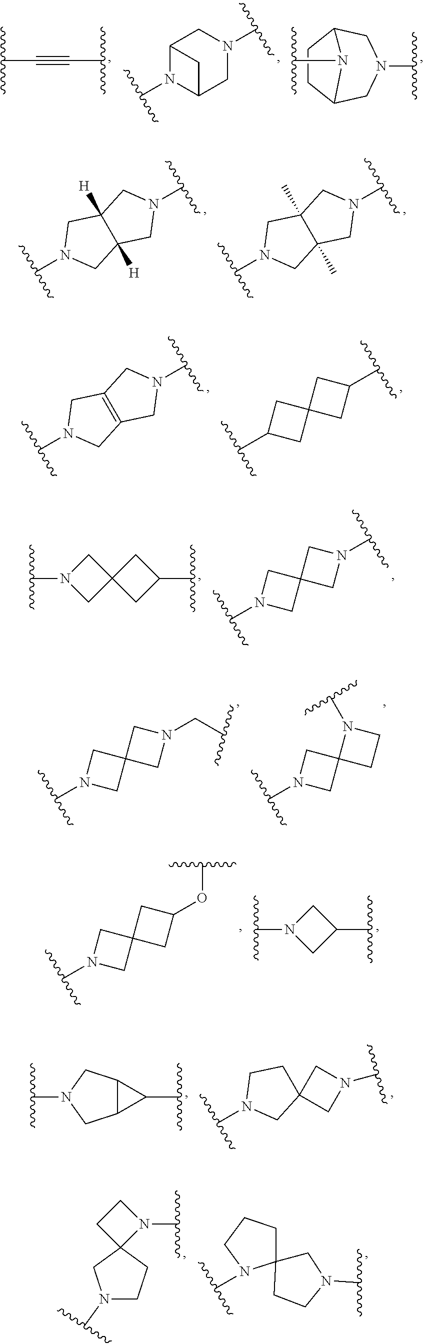

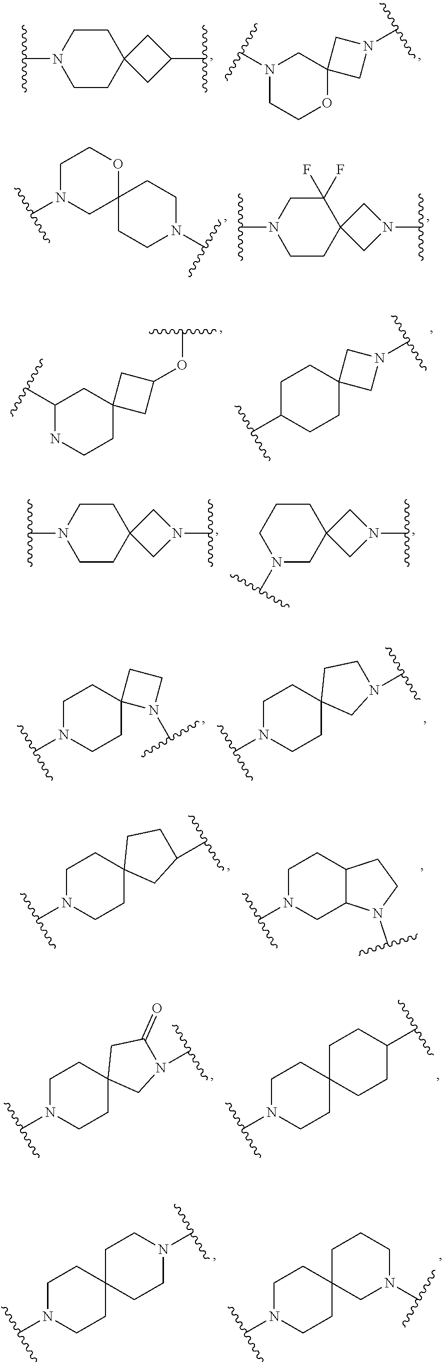

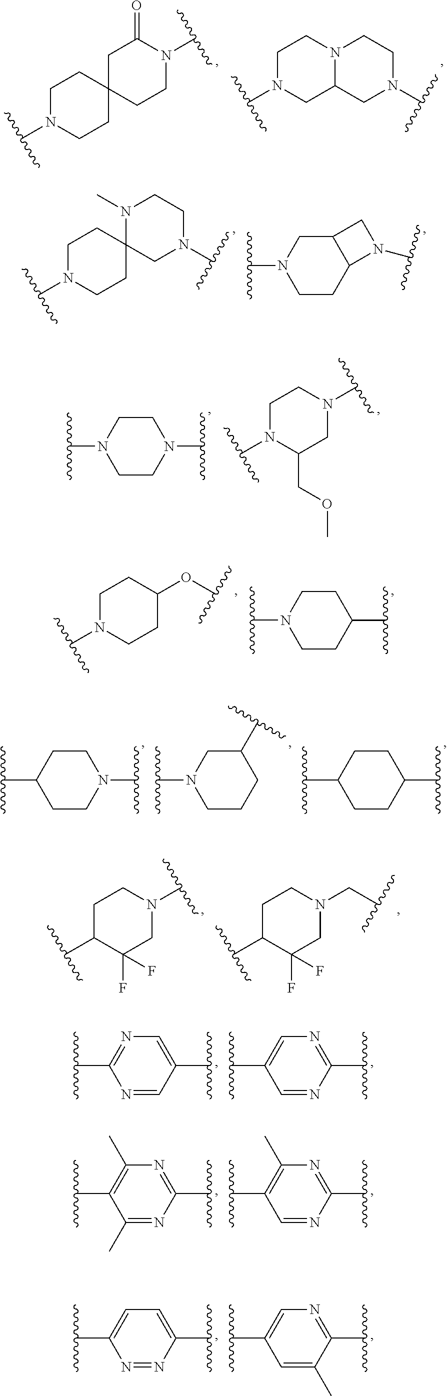

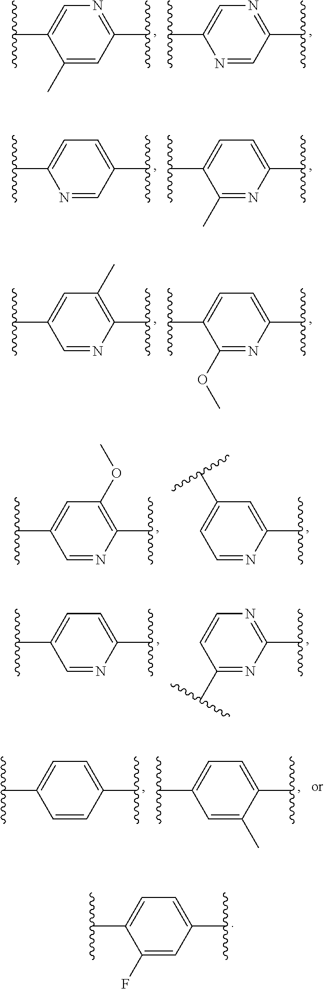

In some embodiments, B² is NR^{N}. In some embodiments, B² is NH. In some embodiments, B² is optionally substituted C₂-C₉ heteroaryl. In some embodiments, B² is:

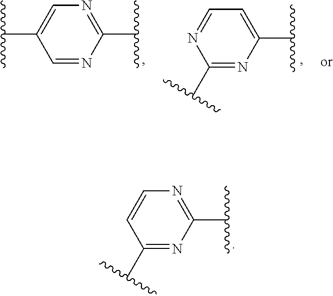 [SMILES: *c1cnc(*)nc1 ; *c1ccnc(*)n1 ; *c1ccnc(*)n1]

In some embodiments, f is 0. In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, the linker has the structure of

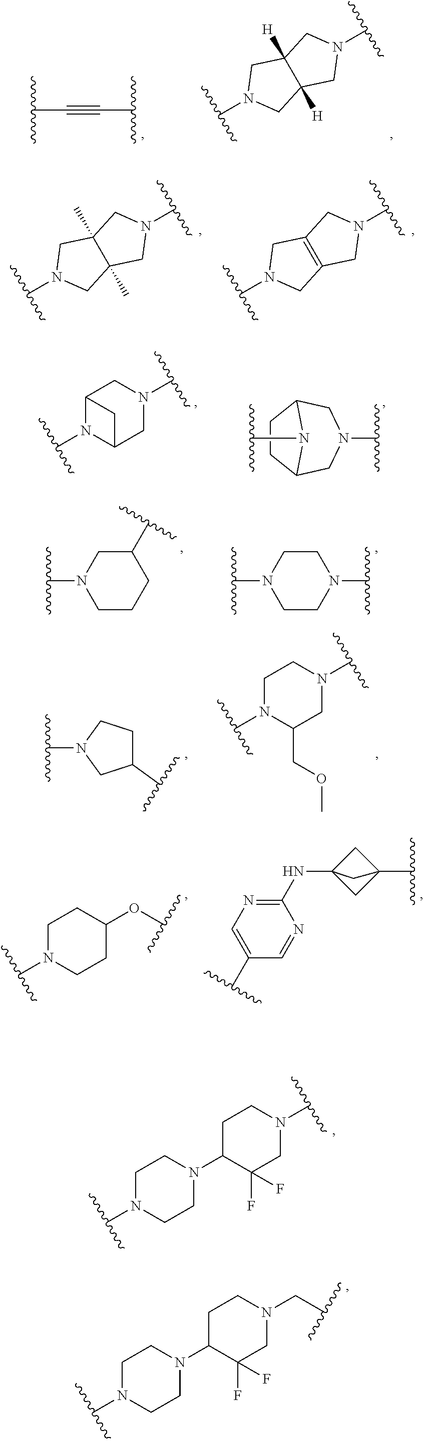

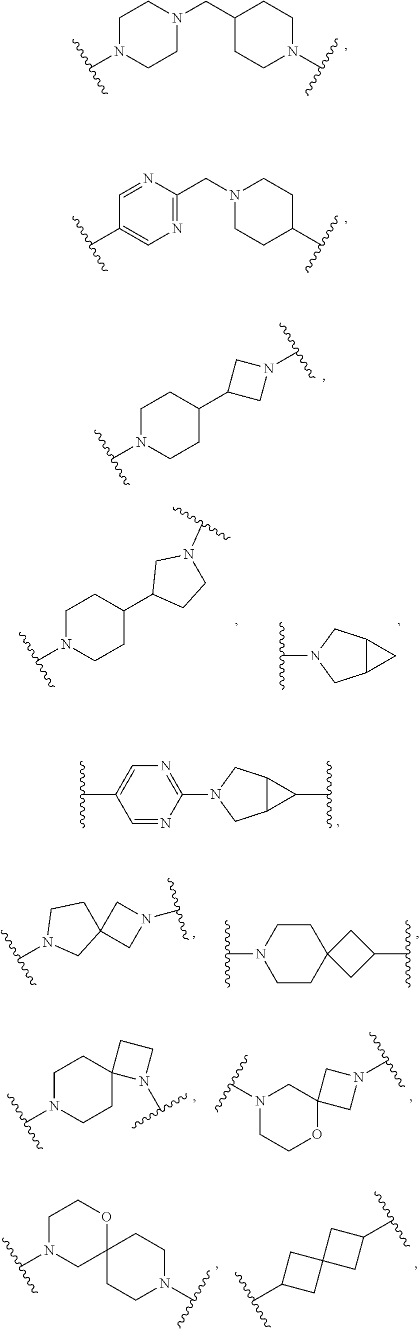

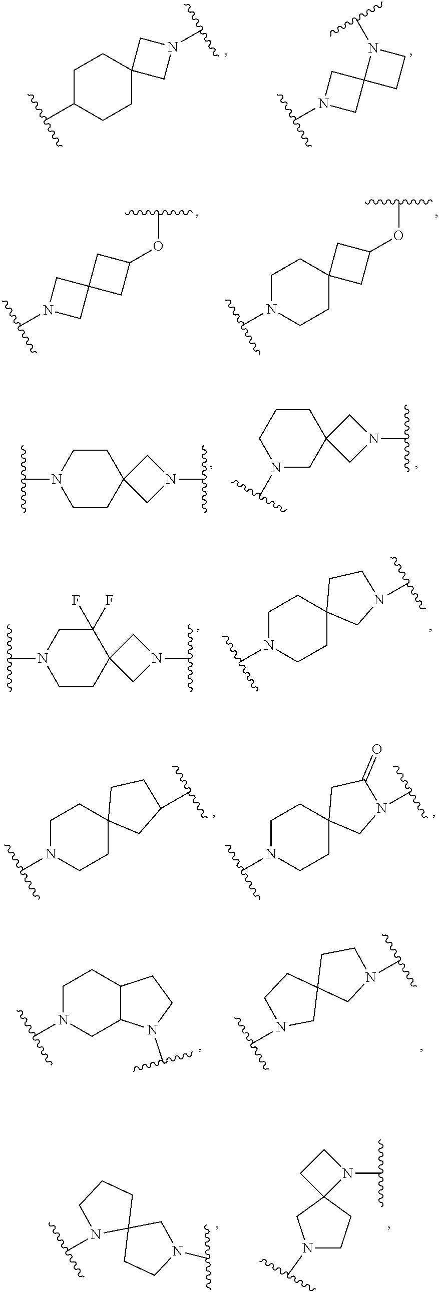

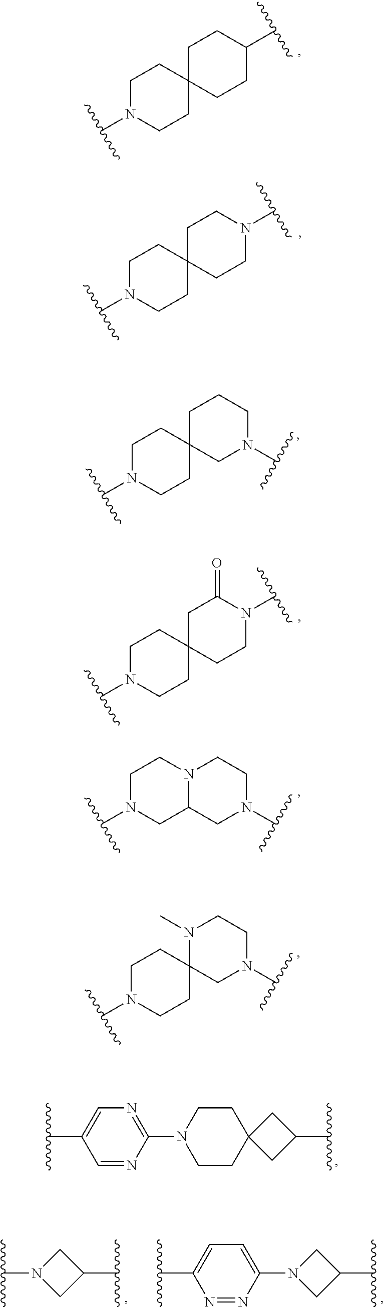

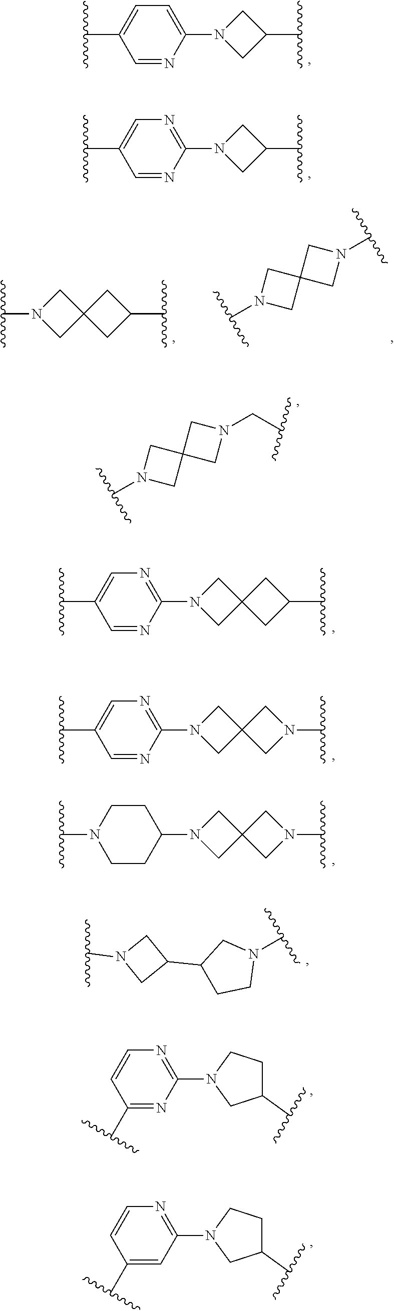

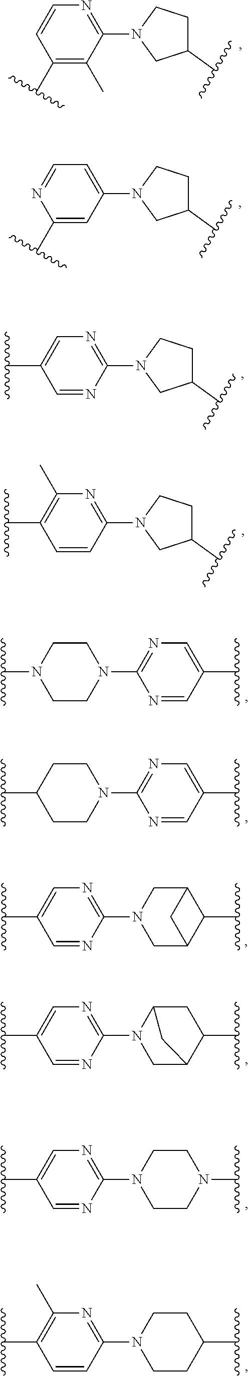

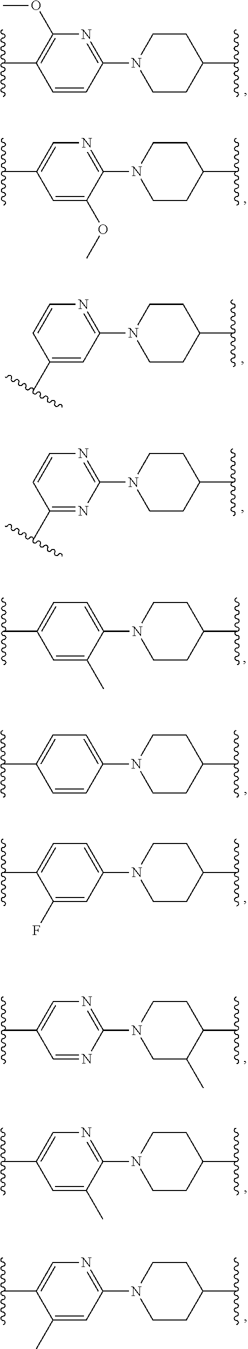

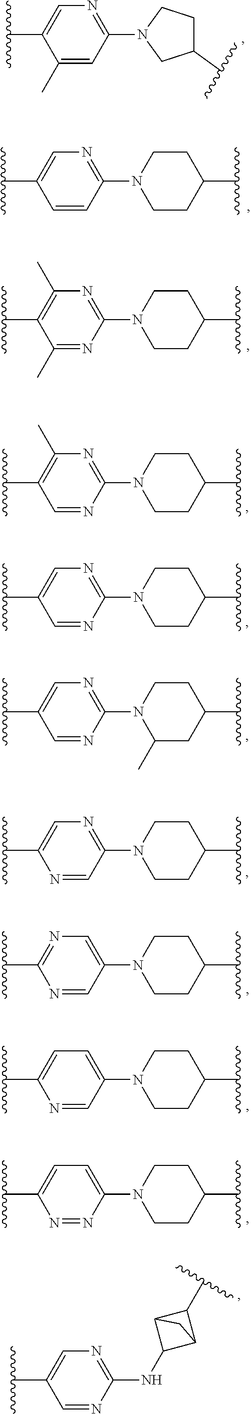

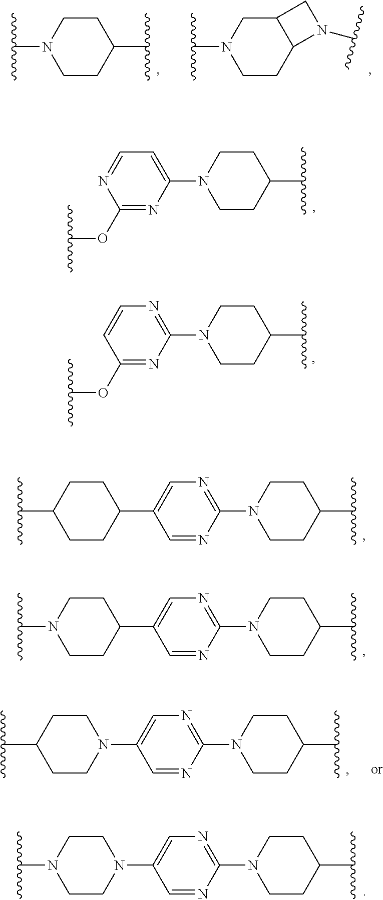

In some embodiments, the shortest chain of atoms connecting two valencies of the linker is 2 to 10 atoms long. In some embodiments, the shortest chain of atoms connecting two valencies of the linker is 6 atoms long.

In some embodiments, the linker has a structure of the linker in any one of compounds 1-4 in Table 1 (e.g., of any of the compounds with a ratio of BRG1 IC₅₀ to BRM IC₅₀ of at least 5 (e.g., at least 7, 10, 15, 20, 25, or 30)). In some embodiments, the linker has a structure of the linker in any one of compounds 1-4 in Table 1 (e.g., of any of the compounds with a BRM IC₅₀ of ++ or better (e.g., +++ or ++++ (e.g., ++++))). In some embodiments, the linker has a structure of the linker in any one of compounds 1-4 in Table 1 (e.g., of any of the compounds with a BRM IC₅₀ of ++ or better (e.g., +++ or ++++ (e.g., ++++)) and with a ratio of BRG1 IC₅₀ to BRM IC₅₀ of at least 5 (e.g., at least 7, 10, 15, 20, 25, or 30)).

In an aspect, the invention features a compound selected from the group consisting of 1-4 in Table 1 and pharmaceutically acceptable salts thereof. In some embodiments, the compound is any one of compounds 1-4 in Table 1 with a ratio of BRG1 IC₅₀ to BRM IC₅₀ of at least 5 (e.g., at least 7, 10, 15, 20, 25, or 30) or a pharmaceutically acceptable salt thereof. In some embodiments, the compound is any one of compounds 1-4 in Table 1 with a BRM IC₅₀ of ++ or better as found in Table 2 (e.g., +++ or ++++ (e.g., ++++)) or a pharmaceutically acceptable salt thereof. In some embodiments, the compound is any one of compounds 1-4 in Table 1 a BRM IC₅₀ of ++ or better as found in Table 2 (e.g., +++ or ++++ (e.g., ++++)) and with a ratio of BRG1 IC₅₀ to BRM IC₅₀ of at least 5 (e.g., at least 7, 10, 15, 20, 25, or 30) or a pharmaceutically acceptable salt thereof.

TABLE 1
Compounds of the Invention

| # | Compound |
|---|---|
| 1 | 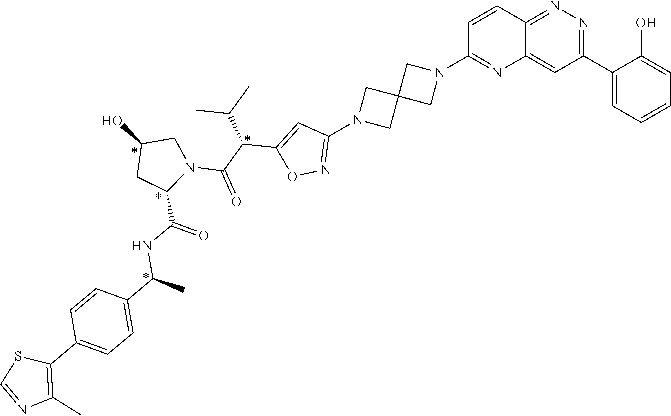 [SMILES: Cc1ncsc1-c1ccc([C@H](C)NC(=O)[C@@H]2C[C@@H](O)CN2C(=O)[C@@H](c2cc(N3CC4(C3)CN(c3ccc5nnc(-c6ccccc6O)cc5n3)C4)no2)C(C)C)cc1] |
| 2 | 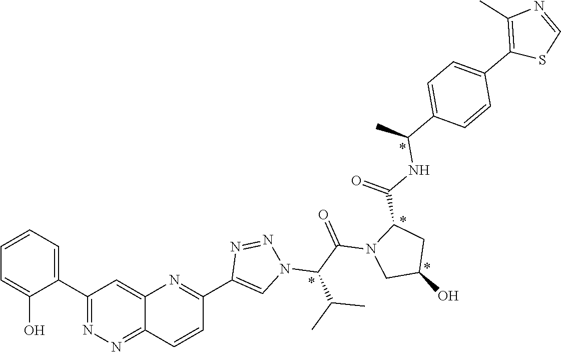 [SMILES: Cc1ncsc1-c1ccc([C@H](C)NC(=O)[C@@H]2C[C@@H](O)CN2C(=O)[C@H](C(C)C)n2cc(-c3ccc4nnc(-c5ccccc5O)cc4n3)nn2)cc1] |
| 3 | 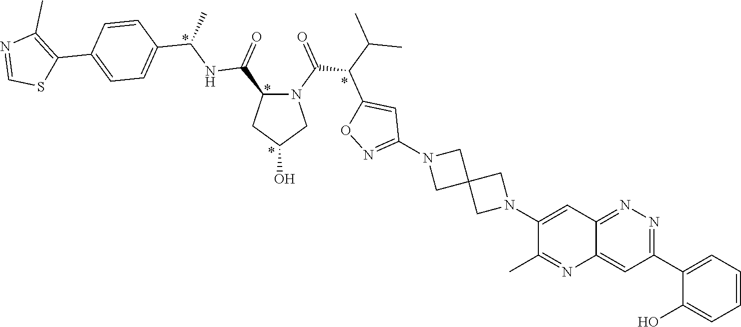 [SMILES: Cc1nc2cc(-c3ccccc3O)nnc2cc1N1CC2(CN(c3cc([C@H](C(=O)N4C[C@H](O)C[C@H]4C(=O)N[C@@H](C)c4ccc(-c5scnc5C)cc4)C(C)C)on3)C2)C1] |
| 4 | 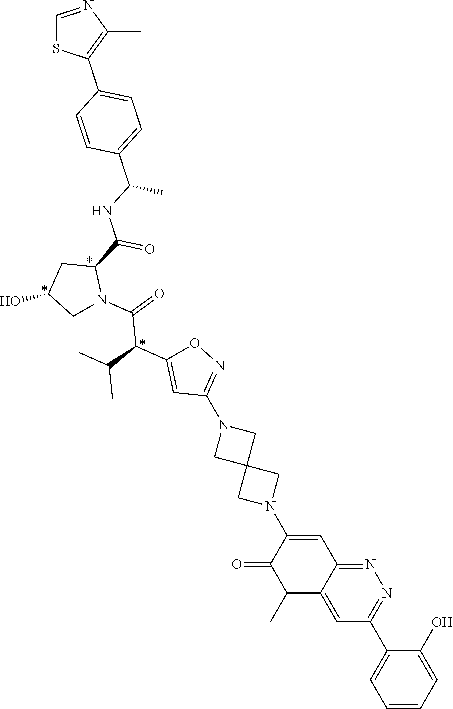 [SMILES: Cc1ncsc1-c1ccc([C@H](C)NC(=O)[C@@H]2C[C@@H](O)CN2C(=O)[C@@H](c2cc(N3CC4(CN(C5=Cc6nnc(-c7ccccc7O)cc6C(C)C5=O)C4)C3)no2)C(C)C)cc1] |

In some embodiments, the compound has a ratio of BRG1 IC₅₀ to BRM IC₅₀ of at least 5. In some embodiments, the compound has a ratio of BRG1 IC₅₀ to BRM IC₅₀ of at least 7. In some embodiments, the compound has a ratio of BRG1 IC₅₀ to BRM IC₅₀ of at least 10. In some embodiments, the compound has a ratio of BRG1 IC₅₀ to BRM IC₅₀ of at least 15. In some embodiments, the compound has a ratio of BRG1 IC₅₀ to BRM IC₅₀ of at least 20. In some embodiments, the compound has a ratio of BRG1 IC₅₀ to BRM IC₅₀ of at least 25. In some embodiments, the compound has a ratio of BRG1 IC₅₀ to BRM IC₅₀ of at least 30.

In an aspect, the invention features a pharmaceutical composition comprising any of the foregoing compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the BAF complex-related disorder is cancer or a viral infection.

In a further aspect, the invention features a method of inhibiting BRM, the method involving contacting a cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In another aspect, the invention features a method of inhibiting BRG1, the method involving contacting the cell with an effective amount of any of the foregoing compounds or a pharmaceutical composition thereof.

In some embodiments, the cell is a cancer cell.

In a further aspect, the invention features a method of inhibiting BRM and BRG1, the method involving contacting the cell with an effective amount of any of the foregoing compounds or a pharmaceutical composition thereof.

In some embodiments, the cell is a cancer cell.

In another aspect, the invention features a method of treating a disorder related to a BRG1 loss of function mutation in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the disorder related to a BRG1 loss of function mutation is cancer. In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In a further aspect, the invention features a method of treating cancer in a subject in need thereof, the method including administering to the subject an effective amount of any of the foregoing compounds (e.g., a BRM/BRG1 dual inhibitor compound or a BRM-selective compound) or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments, the cancer is non-small cell lung cancer. In some embodiments, the cancer is soft tissue sarcoma.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin®, Perjeta®, tamoxifen, Xeloda®, docetaxol, platinum agents such as carboplatin, taxanes such as paclitaxel and docetaxel, ALK inhibitors, MET inhibitors, Alimta®, Abraxane®, 30 Adriamycin®, gemcitabine, Avastin®, Halaven®, neratinib, a PARP inhibitor, ARN810, an mTOR inhibitor, topotecan, Gemzar®, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvirus K*, CMV, varicella-zoster virus), Papillomaviridae family (e.g., Human Papillomavirus (HPV, HPV E1), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvirus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In some embodiments of any of the foregoing aspects, the compound is a BRM-selective compound. In some embodiments, the BRM-selective compound inhibits the level and/or activity of BRM at least 10-fold greater than the compound inhibits the level and/or activity of BRG1 and/or the compound binds to BRM at least 10-fold greater than the compound binds to BRG1. For example, in some embodiments, a BRM-selective compound has an IC₅₀ or IP₅₀ that is at least 10-fold lower than the IC₅₀ or IP₅₀ against BRG1. In some embodiments of any of the foregoing aspects, the compound is a BRM/BRG1 dual inhibitor compound. In some embodiments, the BRM/BRG1 dual inhibitor compound has similar activity against both BRM and BRG1 (e.g., the activity of the compound against BRM and BRG1 with within 10-fold (e.g., less than 5-fold, less than 2-fold). In some embodiments, the activity of the BRM/BRG1 dual inhibitor compound is greater against BRM. In some embodiments, the activity of the BRM/BRG1 dual inhibitor compound is greater against BRG1. For example, in some embodiments, a BRM/BRG1 dual inhibitor compound has an IC₅₀ or IP₅₀ against BRM that is within 10-fold of the IC₅₀ or IP₅₀ against BRG1.

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (IRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments, the cancer harbors a mutation in GNA11. In some embodiments, the cancer harbors a mutation in PLCB4. In some embodiments, the cancer harbors a mutation in CYSLTR2. In some embodiments the cancer harbors a mutation in BAP1. In some embodiments the cancer harbors a mutation in SF3B1. In some embodiments, the cancer harbors a mutation in EIF1AX. In some embodiments the cancer harbors a TFE3 translocation. In some embodiments the cancer harbors a TFEB translocation. In some embodiments, the cancer harbors a MITF translocation. In some embodiments, the cancer harbors an EZH2 mutation. In some embodiments the cancer harbors a SUZ12 mutation. In some embodiments, the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphoinositide 3-kinase inhibitor, proteasome inhibitor, or tyrosine kinase inhibitor.

In some embodiments, the compound of the invention is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the compound of the invention. In some embodiments, the method further comprises administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

In an aspect, the invention provides the use of any of the foregoing compounds (e.g., a BRM/BRG1 dual inhibitor compound or a BRM-selective compound), or pharmaceutically acceptable salts thereof, or any of the foregoing pharmaceutical compositions in the manufacture of a medicament. In some embodiments, the use is as described for the methods described herein.

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. When polycyclic, the aryl group contains 2 or 3 rings. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Unsubstituted arylalkyl groups contain from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each are further substituted with 1, 2, 3, or 4 substituent groups, valency permitting, as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges. A bridged polycycloalkyl group may be unsubstituted or substituted as defined herein for cycloalkyl.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono-di-, or tricyclic radical of 3 to 10, preferably 3 to 6 carbon atoms. The cycloalkyl group may be fully saturated or contain 1 or more double or triple bonds, provided that no ring is aromatic. This term is further exemplified by radicals such as cyclopropyl, cyclobutyl, cyclopentyl, cyclohexyl, cycloheptyl, norbornyl, and adamantyl. The term “cycloalkoxy” as used herein, refers to cycloalkyl-O— groups (e.g., cyclopropoxy and cyclobutoxy).

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group is further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group is further substituted with 1, 2, 3, or 4 substituent groups, valency permitting, as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group is further substituted with 1, 2, 3, or 4 substituent groups, valency permitting, as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to a monocyclic, bicyclic, or tricyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxazolyl, and thiazolyl.

The term “heteroarylalkyl,” as used herein, represents an alkyl group substituted with a heteroaryl group. Unsubstituted heteroarylalkyl groups contain from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each are further substituted with 1, 2, 3, or 4 substituent groups, valency permitting, as defined herein for the respective groups.

The term “heterocyclyl,” as used herein, refers a monocyclic, bicyclic, or tricyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Unsubstituted heterocyclylalkyl groups contain from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each are further substituted with 1, 2, 3, or 4 substituent groups as defined herein for the respective groups.

The term “hydroxyalkyl,” as used herein, represents an alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxy benzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo,” as used herein, represents a divalent oxygen atom (e.g., the structure of oxo may be shown as ═O). For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group).

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will be 1, 2, 3, 4, or 5 substituents present, valency permitting, unless otherwise specified. The 1 to 5 substituents are each, independently, selected from the group consisting of acyl, alkyl (e.g., unsubstituted and substituted, where the substituents include any group described herein, e.g., aryl, halo, hydroxy), alkenyl, alkynyl, aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroalkenyl, heteroalkynyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), carbocyclyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, the substituents are themselves unsubstituted.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, HC, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., 14° C.) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM,” is meant decreasing the level of an activity related to an BRG1 and/or BRM, or a related downstream effect. A non-limiting example of inhibition of an activity of BRG1 and/or BRM is decreasing the level of a BAF complex in a cell. The activity level of BRG1 and/or BRM may be measured using any method known in the art. In some embodiments, an agent which reduces the activity of BRG1 and/or BRM is a small molecule BRG1 and/or BRM degrader.

By “reducing the level of BRG1 and/or BRM,” is meant decreasing the level of BRG1 and/or BRM in a cell or subject. The level of BRG1 and/or BRM may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/ml) or percentage relative to total protein or mRNA in a sample.

As used herein, the term “inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherente, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

The details of one or more embodiments of the invention are set forth in the description below. Other features, objects, and advantages of the invention will be apparent from the description and from the claims.

### DETAILED DESCRIPTION

The present disclosure features compounds useful for the inhibition of BRG1 and optionally BRM. These compounds may be used to modulate the activity of a BAF complex, for example, for the treatment of a BAF-related disorder, such as cancer (e.g., BRG1-loss of function disorders). Exemplary compounds described herein include compounds having a structure according to Formula I, or a pharmaceutically acceptable salt thereof.

The compound of Formula I is;

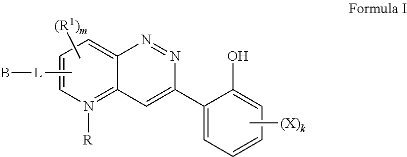

where
m is 0, 1, 2, or 3;
k is 0, 1, or 2;
R is absent or optionally substituted C₁-C₆ alkyl;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl, optionally substituted C₂-C₆ alkynyl, optionally substituted amino, or cyano;
each X is, independently, halo;
L is a linker; and
B is a degradation moiety.

In some embodiments, the compound has the structure of any one of compounds 1-4 in Table 1, or pharmaceutically acceptable salt thereof.

Other embodiments, as well as exemplary methods for the synthesis of production of these compounds, are described herein.

### Pharmaceutical Uses

The compounds described herein are useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders 10 include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment.

Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal1 and calicheamicin omegal1 (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### EXAMPLES

The following abbreviations are used throughout the Examples below.

| | |
|---|---|
| Ac | acetyl |
| ACN or MeCN | acetonitrile |
| AcOH | acetic acid |
| Ac₂O | acetic anhydride |
| aq. | aqueous |
| Boc | tert-butoxycarbonyl |
| Bu or n-Bu | butyl |
| CDI | 1,1′-carbonyldiimidazole |
| DCE or 1,2-DCE | 1,2-dichloroethane |
| DCM | dichloromethane |
| DIAD | diisopropyl azodicarboxylate |
| DIPEA or DIEA | N.N-diisopropylethylamine |
| DMAP | 4-(dimethylamino)pyridine |
| DMB | 2,4-dimethoxybenzyl |
| DME | 1,2-dimethoxyethane |
| DMF | N.N-dimethylformamide |
| DMSO | dimethyl sulfoxide |
| EA or EtOAc | ethyl acetate |
| EDCI | N-(3-dimethylaminopropyl)-N′-ethylcarbodiimide |
| | hydrochloride |
| equiv | equivalents |
| Et₃N or TEA | triethylamine |
| EtOH | ethyl alcohol |
| FA | formic acid |
| h or hr | hour |
| HATU | 1-[bis(dimethylamino)methylene]-1H-1,2,3-triazolo[4,5- |
| | b]pyridinium 3-oxid hexafluorophosphate |
| HOAt | 1-hydroxy-7-azabenzotriazole |
| HOBt or HOBT | 1-hydroxybenzotriazole hydrate |
| iPr | Isopropyl |
| MeOH | methyl alcohol |
| Me₄t-BuXphos | ditert-butyl-[2,3,4,5-tetramethyl-6-(2,4,6- |
| | triisopropylphenyl)phenyl]phosphane |
| min | minute |
| MTBE | tert-butyl methyl ether |
| n-BuLi | n-butylithium |
| NMP | 1-methyl-2-pyrrolidinone |
| OAc | acetate |
| Pd/C | palladium on carbon |
| PDC | pyridinium dichromate |
| PdCl₂(dtbpf) or | dichloro[1,1′-bis(di-t- |
| Pd(dtbpf)Cl₂ | butylphosphino)ferrocene]palladium(II) |
| PdCl₂(dppf) or | [1,1′- |
| Pd(dppf)Cl₂ | bis(diphenylphosphino)ferrocene]dichloropalladium(II) |
| Pd₂(dba)₃ | tris(dibenzylideneacetone)dipalladium(0) |
| Pd(PPh₃)₄ | tetrakis(triphenylphosphine)palladium(0 |
| Pd(PPh₃)₂Cl₂ | dichlorobis(triphenylphosphine)palladium(II) |
| PE | petroleum ether |
| PPh₃ | triphenylphosphine |
| Pr | n-propyl |
| Py | pyridine |
| rac | racemic |
| Rf | retention factor |
| rt. or rt | room temperature |
| sat. | saturated |
| SFC | supercritical fluid chromatography |
| t-Bu | tert-butyl |
| tBuXphos-Pd-G3 or | [2-(2-aminophenyl)phenyl]- |
| tBuXphos Pd G₃ or | methylsulfonyloxypalladium;ditert-butyl-[2-(2,4,6- |
| t-BuXphos-Pd (gen 3) | triisopropylphenyl)phenyl]phosphane |
| TFA | trifluoroacetic acid |
| Tf₂O | trifluoromethanesulfonic anhydride |
| THF | tetrahydrofuran |
| TLC | thin layer chromatography |
| Xantphos-Pd-G3 | [2-(2-aminophenyl)phenyl]-methylsulfonyloxy- |
| | palladium;(5-diphenylphosphanyl-9,9-dimethyl-xanthen- |
| | 4-yl)-diphenyl-phosphane |
| XPhos Pd G3 | (2-Dicyclohexylphosphino-2′,4′,6′-triisopropyl-1,1′- |
| | biphenyl)[2-(2′-amino-1,1′-biphenyl)]palladium(II) |
| | methanesulfonate |

### Example 1. Preparation of Preparation of (2S,4R)-4-hydroxy-1-[(2R)-2-(3-{6-[3-(2-hydroxyphenyl)-6-methylpyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 3)

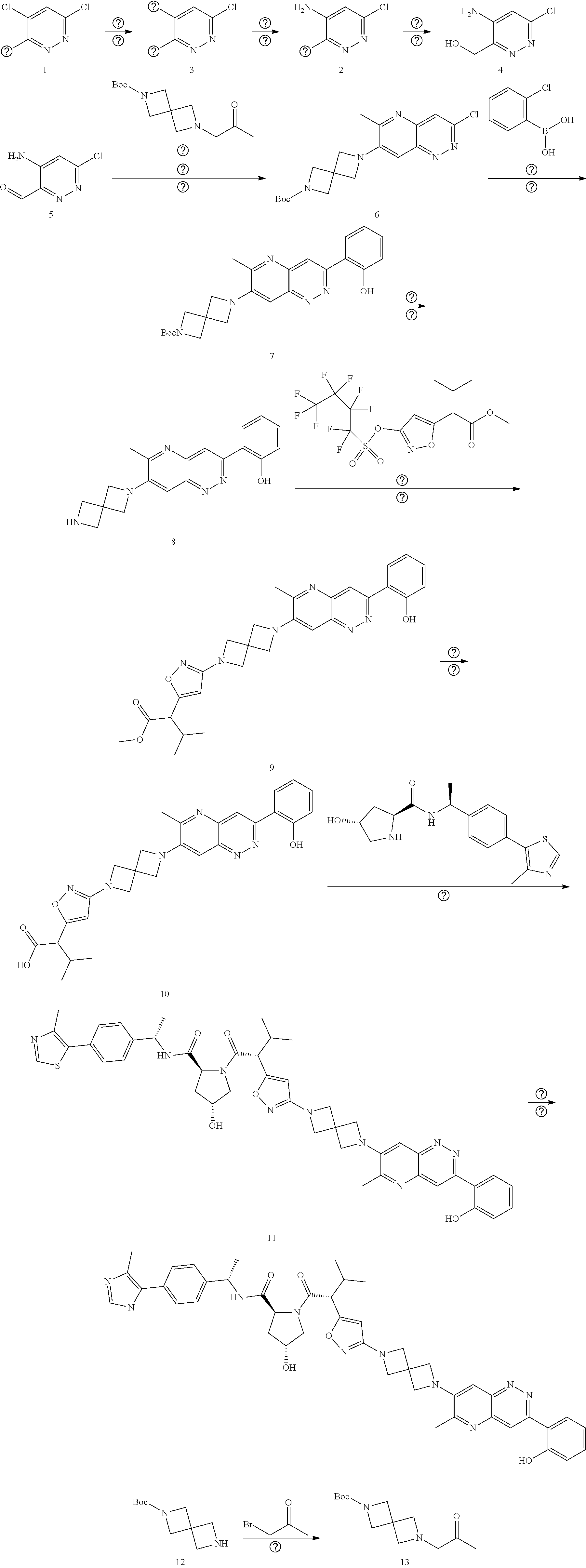

### Step 1: Preparation of methyl 6-chloro-4-{[(2,4-dimethoxyphenyl)methyl]amino}pyridazine-3-carboxylate (Intermediate 2)

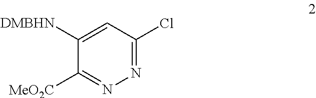

A mixture of methyl 4,6-dichloropyridazine-3-carboxylate (10 g, 48.307 mmol, 1 equiv), 1-(2,4-dimethoxyphenyl) methanamine (8.08 g, 48.307 mmol, 1 equiv) and DIEA (12.49 g, 96.614 mmol, 2 equiv) in NMP (50 mL) was stirred for 2 h at 60° C. Desired product could be detected by LCMS. The resulting mixture was diluted with brine (400 mL). The resulting mixture was extracted with EtOAc (3×300 mL). The combined organic layers were washed with brine (3×100 mL), dried over anhydrous Na₂SO₄. After filtration, the filtrate was concentrated under reduced pressure to afford Intermediate 2 (15.6 g, 95.61%) as a yellow solid. LCMS (ESI) m/z: [M+H]⁺=338.

### Step 2: Preparation of methyl 4-amino-6-chloropyridazine-3-carboxylate (Intermediate 3)

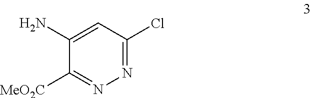 [SMILES: COC(=O)c1nnc(Cl)cc1N]

A mixture of Intermediate 2 (5 g, 14.803 mmol, 1 equiv) in TFA (10 mL) was stirred for 1 h at 80° C. under nitrogen atmosphere. Desired product could be detected by LCMS. The resulting mixture was diluted with MeCN (40 mL). The precipitated solids were collected by filtration and washed with MeCN (3×10 mL). The resulting mixture was concentrated under reduced pressure to afford Intermediate 3 (4.1 g, TFA salt) as a light purple solid. LCMS (ESI) m/z [M+H]⁺=188.

### Step 3: Preparation of (4-amino-6-chloropyridazin-3-yl) methanol (Intermediate 4)

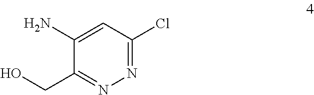

A mixture of Intermediate 3 (4 g, 21.324 mmol, 1 equiv) and CaCl₂) (4.73 g, 42.648 mmol, 2 equiv) in EtOH (40 mL) was added NaBH₄ (1.61 g, 42.648 mmol, 2 equiv) at 0° C. The resulting mixture was stirred for 2 h at room temperature under nitrogen atmosphere. Desired product could be detected by LCMS. The reaction was quenched by the addition of Water (100 mL) at room temperature. The resulting mixture was filtered, the filter cake was washed with MeCN (3×100 mL). The filtrate was concentrated under reduced pressure to afford Intermediate 4 (960 mg, 28.21%) as a white solid. LCMS (EST) m/z: [M+H]⁺=160.

### Step 4: Preparation of 4-amino-6-chloropyridazine-3-carbaldehyde (Intermediate 5)

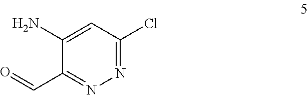

A mixture of Intermediate 4 and MnO₂ (1.55 g, 17.862 mmol, 3 equiv) in DCM (10 mL) was stirred for 4 h at room temperature. Desired product could be detected by LCMS. The resulting mixture was filtered, the filter cake was washed with MeOH (3×50 mL). The filtrate was concentrated under reduced pressure to afford Intermediate 5 (100 mg, 10.66%) as a white solid. LCMS (ESI) m/z: [M+H]⁺=158.

### Step 5: Preparation of tert-butyl 6-{3-chloro-6-methylpyrido[3,2-c]pyridazin-7-yl}-2,6-diazaspiro[3.3]heptane-2-carboxylate (Intermediate 6)

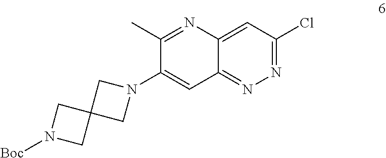 [SMILES: Cc1nc2cc(Cl)nnc2cc1N1CC2(CN(C(=O)OC(C)(C)C)C2)C1]

A mixture of Intermediate 5 (150 mg, 0.952 mmol, 1 equiv), tert-butyl 6-(2-oxopropyl)-2,6-diazaspiro[3.3]heptane-2-carboxylate (266.34 mg, 1.047 mmol, 1.1 equiv) and Pyridine (150.61 mg, 1.904 mmol, 2 equiv) in EtOH (3 mL) was stirred for 5 h at 60° C. under nitrogen atmosphere. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 6 (70 mg, 19.56%) as a brown yellow solid. LCMS (ESI) m/z: [M+H]⁺=376.

### Step 6: Preparation of tert-butyl 6-[3-(2-hydroxyphenyl)-6-methylpyrido[3,2-c]pyridazin-7-yl]-2,6-diazaspiro[3.3]heptane-2-carboxylate (Intermediate 7)

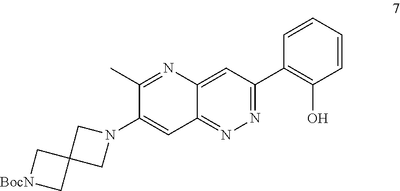 [SMILES: Cc1nc2cc(-c3ccccc3O)nnc2cc1N1CC2(CN(C(=O)OC(C)(C)C)C2)C1]

A mixture of Intermediate 6 (65 mg, 0.173 mmol, 1 equiv), 2-hydroxyphenylboronic acid (71.56 mg, 0.519 mmol, 3 equiv), XPhos Pd G3 (29.28 mg, 0.035 mmol, 0.2 equiv) and Cs₂CO₃ (169.04 mg, 0.519 mmol, 3 equiv) in dioxane (2 mL) and H₂O (0.4 mL) was stirred for 2 h at 80° C. under nitrogen atmosphere. Desired product could be detected by LCMS. The resulting mixture was concentrated under reduced pressure. The residue was purified by silica gel column chromatography, eluted with PE/EA (1:1) to afford Intermediate 7 (72.6 mg, 96.84%) as a yellow solid. LCMS (ESI) m/z: [M+H]⁺=434.

### Step 7: Preparation of 2-(7-{2,6-diazaspiro[3.3]heptan-2-yl}-6-methylpyrido[3,2-c]pyridazin-3-yl)phenol (Intermediate 8)

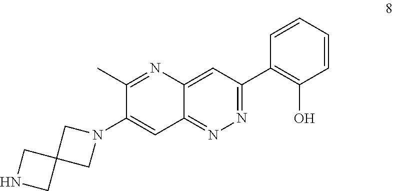 [SMILES: Cc1nc2cc(-c3ccccc3O)nnc2cc1N1CC2(CNC2)C1]

A mixture of Intermediate 7 (67.6 mg, 0.156 mmol, 1 equiv) and TEA (1 mL) in DCM (3 mL) was stirred for 0.5 h at room temperature. Desired product could be detected by LCMS. The resulting mixture was concentrated under reduced pressure to afford Intermediate 8 (75 mg, TFA salt) as a dark red solid. LCMS (ESI) m/z: [M+H]⁺=334.

### Step 8: Preparation of methyl 2-(3-{6-[3-(2-hydroxyphenyl)-6-methylpyrido[3,2-c]pyridazin-7-yl]-2,6-diazaspiro[3.3]heptan-2-yl}-1,2-oxazol-5-yl)-3-methylbutanoate (Intermediate 9)

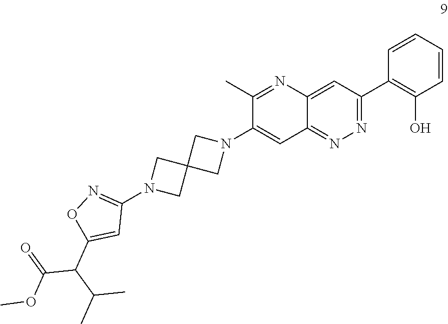 [SMILES: COC(=O)C(c1cc(N2CC3(C2)CN(c2cc4nnc(-c5ccccc5O)cc4nc2C)C3)no1)C(C)C]

A mixture of Intermediate 8 (70 mg, 0.210 mmol, 1 equiv), methyl 3-methyl-2-{3-[(1,1,2,2,3,3,4,4,4-nonafluorobutanesulfonyl)oxy]-1,2-oxazol-5-yl}butanoate (202.10 mg, 0.420 mmol, 2 equiv) and DIEA (81.41 mg, 0.630 mmol, 3 equiv) in DMSO (3 mL) was stirred for 1 h at 120° C. under nitrogen atmosphere. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 9 (70 mg, 64.79%) as a yellow solid. LCMS (ESI) m/z: [M+H]⁺=515.

### Step 9: Preparation of 2-(3-{6-[3-(2-hydroxyphenyl)-6-methylpyrido[3,2-c]pyridazin-7-yl]-2,6-diazaspiro[3.3]heptan-2-yl}-1,2-oxazol-5-yl)-3-methylbutanoic acid (Intermediate 9)

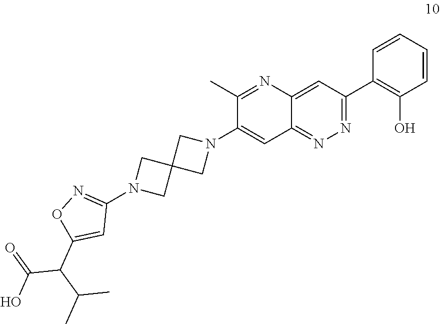 [SMILES: Cc1nc2cc(-c3ccccc3O)nnc2cc1N1CC2(CN(c3cc(C(C(=O)O)C(C)C)on3)C2)C1]

A mixture of Intermediate 9 (65 mg, 0.126 mmol, 1 equiv) and LiOH·H₂O (26.50 mg, 0.630 mmol, 5 equiv) in MeOH (1 mL), THF (1 mL) and H₂O (0.5 mL) was stirred for 1 h at room temperature. Desired product could be detected by LCMS. The resulting mixture was concentrated under reduced pressure to afford Intermediate 10 (72.3 mg, crude) as a dark red solid. LCMS (ESI) m/z: [M+H]⁺=501.

### Step 10: Preparation of (2S,4R)-4-hydroxy-1-[2-(3-{6-[3-(2-hydroxyphenyl)-6-methylpyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Intermediate 11)

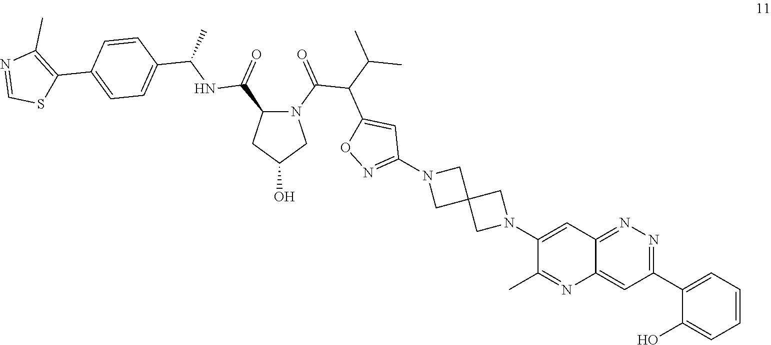 [SMILES: Cc1nc2cc(-c3ccccc3O)nnc2cc1N1CC2(CN(c3cc(C(C(=O)N4C[C@H](O)C[C@H]4C(=O)N[C@@H](C)c4ccc(-c5scnc5C)cc4)C(C)C)on3)C2)C1]

A mixture of Intermediate 10 (70 mg, 0.140 mmol, 1 equiv), (2S,4R)-4-hydroxy-N-[(1S)-1-[4-(4-methyl-1,3-thiazol-5-yl)phenyl]ethyl]pyrrolidine-2-carboxamide (46.35 mg, 0.140 mmol, 1 equiv), PyBOP (87.33 mg, 0.168 mmol, 1.2 equiv) and DIEA (54.22 mg, 0.420 mmol, 3 equiv) in DMF (2 mL) was stirred for 1 h at room temperature. Desired product could be detected by LCMS. The crude product was purified by Prep-HPLC with the following conditions (Column: XBridge Shield RP18 OBD Column, 30*150 mm, 5 μm; Mobile Phase A: Water (10 mmol/L NH₄HCO₃+0.05% NH₃·H₂O), Mobile Phase B: ACN; Flow rate: 60 mL/min; Gradient: 31% B to 51% B in 10 min; Wave Length: 254 nm/220 nm to afford Intermediate 11 (23 mg, 20.21%) as a yellow solid. LCMS (ESI) m/z: [M+H]⁺=814.

### Step 11: Preparation of (2S,4R)-4-hydroxy-1-[(2R)-2-(3-{6-[3-(2-hydroxyphenyl)-6-methylpyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 3)

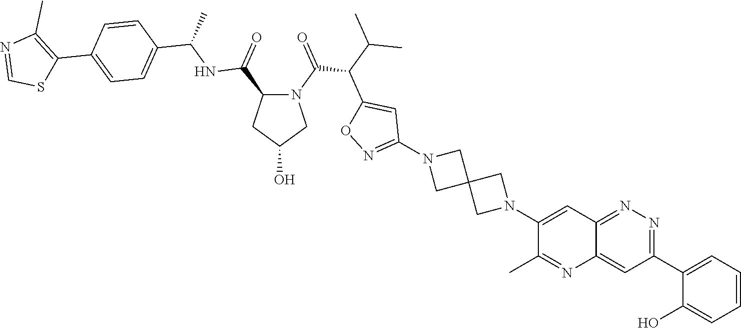 [SMILES: Cc1nc2cc(-c3ccccc3O)nnc2cc1N1CC2(CN(c3cc([C@H](C(=O)N4C[C@H](O)C[C@H]4C(=O)N[C@@H](C)c4ccc(-c5scnc5C)cc4)C(C)C)on3)C2)C1]

The Intermediate 11 was purified by Chiral-HPLC with the following conditions (Column: CHIRALPAK IA 2*25 cm. 5 μm; Mobile Phase A: MtBE (10 mM NH₃-MeOH), Mobile Phase B: ACN:DCM=2:1 (0.1% 2M NH-MeOH); Flow rate: 20 mL/min; Gradient: isocratic 30; Wave Length: 212/262 nm; RT1(min): 9; RT2(min): 17.25; Sample Solvent: MeOH:DCM=1:1-HPLC; Injection Volume: 1.2 mL to afford Compound 3 (6.4 mg, 31.74%) as a yellow solid. ¹H NMR (400 MHz, DMSO-d6) δ 12.43 (s, 1H), 9.00 (s, 1H), 8.69 (s, 1H), 8.43 (d, J=7.7 Hz, 1H), 8.19-8.12 (m, 1H), 7.51-7.28 (m, 6H), 7.06-6.96 (m, 2H), 5.88 (s, 1H), 5.07 (dd, J=43.2, 3.2 Hz, 1H), 4.88 (dp, 1H), 4.44 (s, 4H), 4.37 (t, J=7.9 Hz, 1H), 4.29 (s, 1H), 4.12 (s, 4H), 3.75-3.67 (m, 1H), 3.65-3.55 (m, 1H), 3.53-3.40 (m, 1H), 2.70 (s, 3H), 2.47 (s, 3H), 2.24-2.19 (m, 1H), 2.08-1.98 (m, 1H), 1.84-1.73 (m, 1H), 1.43 (dd, J=33.4, 7.0 Hz, 3H), 1.01-0.93 (m, 3H), 0.82 (dd, J=14.7, 6.7 Hz, 3H). LCMS (ESI) m/z: [M+H]⁺=814.35.

### Step 12: Preparation of tert-butyl 6-(2-oxopropyl)-2,6-diazaspiro[3.3]heptane-2-carboxylate (Intermediate 13)

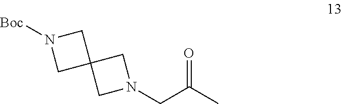 [SMILES: CC(=O)CN1CC2(C1)CN(C(=O)OC(C)(C)C)C2]

A mixture of tert-butyl 2,6-diazaspiro[3.3]heptane-2-carboxylate (5 g, 25.219 mmol, 1 equiv), bromoacetone (3.8 g, 27.741 mmol, 1.1 equiv) and K₂CO₃ (6.97 g, 50.438 mmol, 2 equiv) in MeCN (50 mL) was stirred for 2 h at room temperature. Desired product could be detected by LCMS. The residue was purified by reversed-phase flash chromatography with the following conditions: column, C18 silica gel; mobile phase, MeCN in Water (0.1% FA), 0% to 20% gradient in 20 min to afford Intermediate 13 (580 mg, 9.04%) as a brown oil. LCMS (ESI) m/z: [M+H]⁺=255.

### Preparation of Preparation of (2S,4R)-4-hydroxy-1-[(2R)-2-(3-{6-[3-(2-hydroxyphenyl)pyrido[3,2-c]pyridazin-6-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 1)

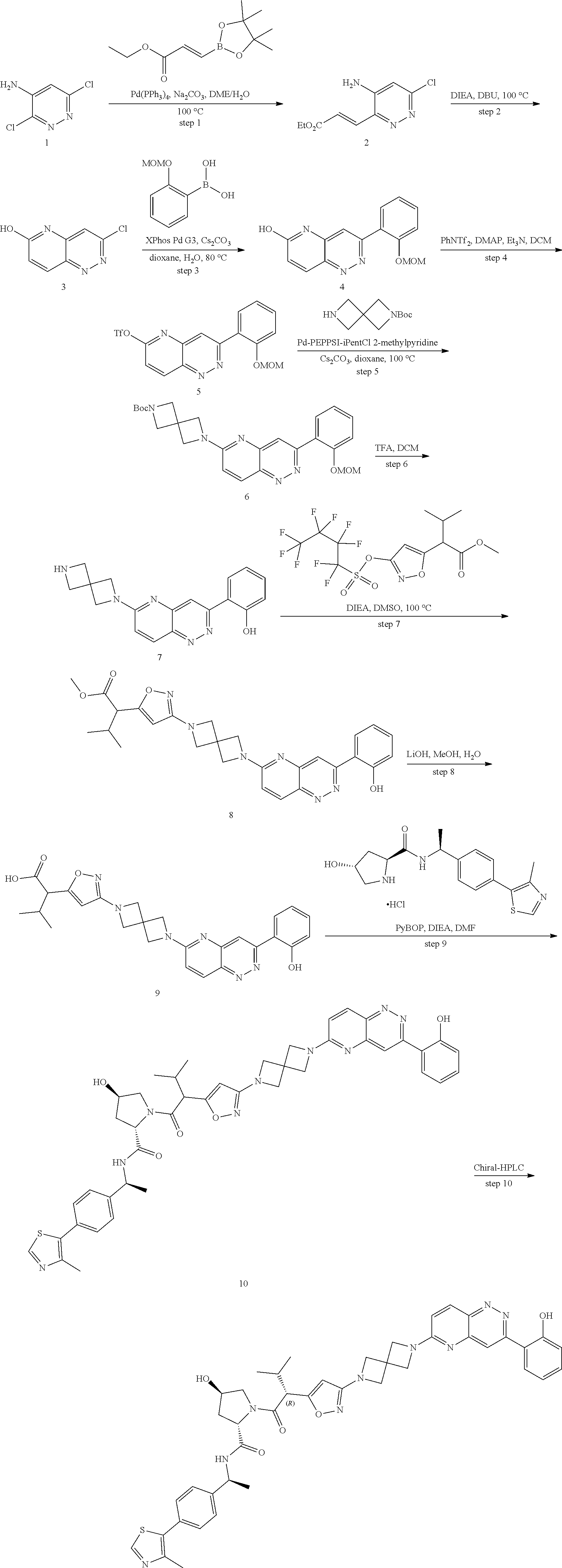

### Step 1: Preparation of methyl ethyl(2E)-3-(4-amino-6-chloropyridazin-3-yl)prop-2-enoate (Intermediate 2)

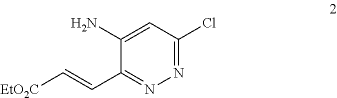 [SMILES: CCOC(=O)/C=C/c1nnc(Cl)cc1N]

A mixture of 3,6-dichloropyridazin-4-amine (5 g, 30.490 mmol, 1 equiv), ethyl(2E)-3-(4,4,5,5-tetramethyl-1,3,2-dioxaborolan-2-yl)prop-2-enoate (8.27 g, 36.588 mmol, 1.2 equiv), Pd(PPb₃)₄ (3.52 g, 3.049 mmol, 0.1 equiv) and Na₂CO; (6.46 g, 60.980 mmol, 2 equiv) in DME (30 mL) and H₂O (10 mL) was stirred for overnight at 100° C. under nitrogen atmosphere. Desired product could be detected by LCMS. The resulting mixture was diluted with water (300 mL). The resulting mixture was extracted with EtOAc (3×300 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (1:3) to afford Intermediate 2 (6.5 g, 93.65%) as a yellow solid. LCMS (EST) m/z: [M+H]⁺=228.

### Step 2: Preparation of 3-chloropyrido[3,2-c]pyridazin-6-ol (Intermediate 3)

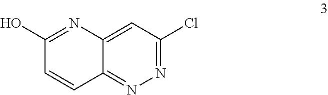 [SMILES: Oc1ccc2nnc(Cl)cc2n1]

A mixture of Intermediate 2 (3 g, 13.178 mmol, 1 equiv) and DIBA (3 mL) in DBU (15 mL) was stirred for overnight at 100° C. under nitrogen atmosphere. Desired product could be detected by LCMS. The residue was purified by silica gel column chromatography, eluted with CH₂Cl₂/MeOH (19:1) to afford Intermediate 3 (3.2 g, crude) as a yellow solid. LCMS (ESI) m/z [M+H]⁺=182.

### Step 3: Preparation of 3-[2-(methoxymethoxy)phenyl]pyrido[3,2-c]pyridazin-6-ol (Intermediate 4)

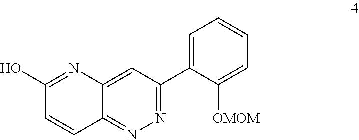

A mixture of Intermediate 3 (1 g, 5.507 mmol, 1 equiv), 2-(methoxymethoxy)phenylboronic acid (1.50 g, 8.261 mmol, 1.5 equiv), XPhos Pd G3 (466.16 mg, 0.551 mmol, 0.1 equiv) and Cs₂CO₃ (2.69 g, 8.261 mmol, 1.5 equiv) in dioxane (10 mL) and H₂O (2 mL) was stirred for 2 h at 80° C. under nitrogen atmosphere. Desired product could be detected by LCMS. The residue was purified by silica gel column chromatography, eluted with CH₂Cl₂/MeOH (20:1) to afford Intermediate 4 (1.5 g, 96.15%) as a yellow solid. LCMS (ESI) m/z: [M+H]⁺=284.

### Step 4: Preparation of 3-[2-(methoxymethoxy)phenyl]pyrido[3,2-c]pyridazin-6-yl trifluoromethanesulfonate (Intermediate 5)

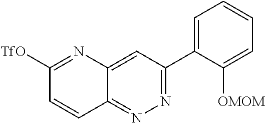

A mixture of Intermediate 4 (1.5 g, 5.295 mmol, 1 equiv), 1,1,1-trifluoro-N-phenyl-N-trifluoromethanesulfonylmethanesulfonamide (2.84 g, 7.942 mmol, 1.5 equiv), DMAP (64.69 mg, 0.529 mmol, 0.1 equiv) and Et₃N (1.61 g, 15.885 mmol, 3 equiv) in DCM (10 mL) was stirred for 1 h at room temperature. Desired product could be detected by LCMS. The resulting mixture was concentrated under reduced pressure. The residue was purified by silica gel column chromatography, eluted with PE/EA (3:1) to afford Intermediate 5 (690 mg, 31.37%) as a brown oil. LCMS (ESI) m/z: [M+H]⁺=416.

### Step 5: Preparation of tert-butyl 6-{3-[2-(methoxymethoxy)phenyl]pyrido[3,2-c]pyridazin-6-yl}-2,6-diazaspiro[3.3]heptane-2-carboxylate (Intermediate 6)

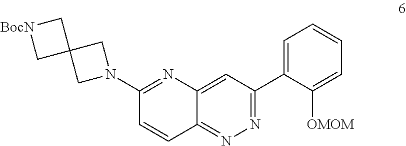

A mixture of Intermediate 5 (500 mg, 1.204 mmol, 1 equiv), tert-butyl 2,6-diazaspiro[3.3]heptane-2-carboxylate (358.02 mg, 1.806 mmol, 1.5 equiv), Pd-PEPPSI-IPentCl 2-methylpyridine (o-picoline (50.63 mg, 0.060 mmol, 0.05 equiv) and Cs₂CO₃ (1.18 g, 3.612 mmol, 3 equiv) in dioxane (0.5 mL) was stirred for 2 h at 100° C. under nitrogen atmosphere. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 6 (350 mg, crude) as a brown oil. LCMS (ESI) m/z: [M+H]⁺=464.

### Step 6: Preparation of 2-(6-{2,6-diazaspiro[3.3]heptan-2-yl}pyrido[3,2-c]pyridazin-3-yl)phenol (Intermediate 7)

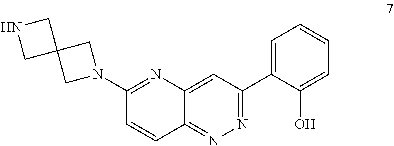 [SMILES: Oc1ccccc1-c1cc2nc(N3CC4(CNC4)C3)ccc2nn1]

A mixture of Intermediate 6 (340 mg, 0.733 mmol, 1 equiv) and TFA (1 mL) in DCM (3 mL) was stirred for 2 h at room temperature. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 7 (35 mg, 14.94%) as a yellow solid. LCMS (ESI) m/z: [M+H]⁺=320.

### Step 7: Preparation of methyl 2-(3-{6-[3-(2-hydroxyphenyl)pyrido[3,2-c]pyridazin-6-yl]-2,6-diazaspiro[3.3]heptan-2-yl}-1,2-oxazol-5-yl)-3-methylbutanoate (Intermediate 8)

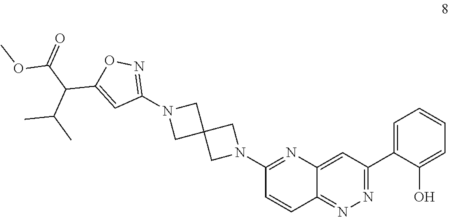 [SMILES: COC(=O)C(c1cc(N2CC3(C2)CN(c2ccc4nnc(-c5ccccc5O)cc4n2)C3)no1)C(C)C]

A mixture of Intermediate 7 (35 mg, 0.110 mmol, 1 equiv), methyl 3-methyl-2-{3-[(1,1,2,2,3,3,4,4,4-nonafluorobutanesulfonyl)oxy]-1,2-oxazol-5-yl}butanoate (105.49 mg, 0.220 mmol, 2 equiv) and DIEA (42.49 mg, 0.330 mmol, 3 equiv) in DMSO (1 mL) was stirred for 2 h at 100° C. under nitrogen atmosphere. Desired product could be detected by LCMS. The resulting mixture was diluted with water (10 mL). The resulting mixture was extracted with EtOAc (3×20 mL). The combined organic layers were washed with brine (3×10 mL), dried over anhydrous Na₂SO₄. After filtration, the filtrate was concentrated under reduced pressure to afford Intermediate 8 (106 mg, crude) as a brown solid. LCMS (ESI) m/z: [M+H]⁺=501.

### Step 8: Preparation of 2-(3-{6-[3-(2-hydroxyphenyl)pyrido[3,2-c]pyridazin-6-yl]-2,6-diazaspiro[3.3]heptan-2-yl}-1,2-oxazol-5-yl)-3-methylbutanoic acid (Intermediate 9)

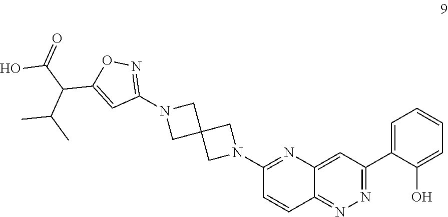 [SMILES: CC(C)C(C(=O)O)c1cc(N2CC3(C2)CN(c2ccc4nnc(-c5ccccc5O)cc4n2)C3)no1]

A mixture of Intermediate 8 (30 mg, 0.060 mmol, 1 equiv) and LiOH·H₂O (7.18 mg, 0.300 mmol, 5 equiv) in MeOH (1 mL), THE (1 mL) and H₂O (0.5 mL) was stirred for 1 h at room temperature. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 9 (16 mg, 54.87%) as a yellow solid. LCMS (ESI) m/z: [M+H]⁺=487.

### Step 9: Preparation of (2S,4R)-4-hydroxy-1-[2-(3-{6-[3-(2-hydroxyphenyl)pyrido[3,2-c]pyridazin-6-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Intermediate 9)

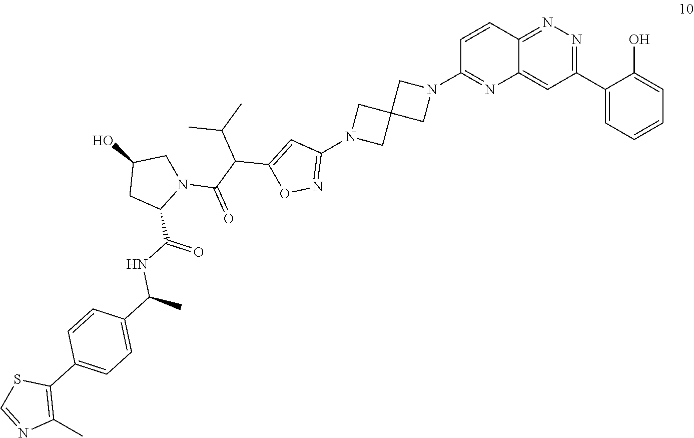 [SMILES: Cc1ncsc1-c1ccc([C@H](C)NC(=O)[C@@H]2C[C@@H](O)CN2C(=O)C(c2cc(N3CC4(C3)CN(c3ccc5nnc(-c6ccccc6O)cc5n3)C4)no2)C(C)C)cc1]

A mixture of Intermediate 9 (11 mg, 0.023 mmol, 1 equiv), (2S,4R)-4-hydroxy-N-[(1S)-1-[4-(4-methyl-1,3-thiazol-5-yl)phenyl]ethyl]pyrrolidine-2-carboxamide (11.24 mg, 0.035 mmol, 1.5 equiv), PyBOP (17.65 mg, 0.035 mmol, 1.5 equiv) and DIEA (11.69 mg, 0.092 mmol, 4 equiv) in DMF (1 mL) was stirred for 1 b at room temperature. Desired product could be detected by LCMS. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₃·H₂O), Mobile Phase B: ACN; Flow rate: 60 mL/min; Gradient: 32% B to 52% B in 10 min; Wave Length: 254 nm/220 nm to afford Intermediate 10 (10 mg, 55.29%) as a yellow solid. LCMS (ESI) m/z: [M+H]⁺=800.

### Step 10: Preparation of (2S,4R)-4-hydroxy-1-[(2R)-2-(3-{6-[3-(2-hydroxyphenyl)pyrido[3,2-c]pyridazin-6-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 1)

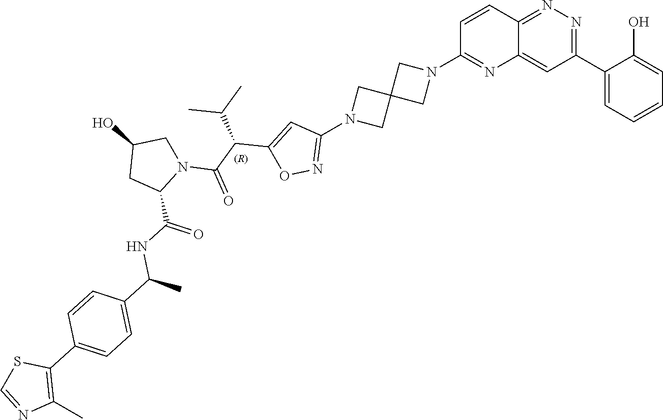

The Intermediate 10 (10 mg) was purified by Chiral-HPLC with the following conditions (Column: CHIRALPAK ID, 2*25 cm, 5 μm; Mobile Phase A: MtBE (0.5% 2M NH₃-MeOH)-HPLC, Mobile Phase B: MeOH-HPLC; Flow rate: 20 mL/min; Gradient: isocratic 30; Wave Length: 276/214 nm; RT1(min): 9.766; RT2(min): 16.448; Sample Solvent: MeOH:DCM=4:1; Injection Volume: 1.5 mL) to afford Compound 1 (3.3 mg, 32.87%) as an off-white solid. ¹H NMR (400 MHz, DMSO-d6) δ 13.24 (s, 1H), 8.99 (s, 1H), 8.43-8.30 (m, 3H), 8.17 (dd, J=8.0, 1.7 Hz, 1H), 7.51-7.41 (m, 2H), 7.45-7.31 (m, 3H), 7.13 (d, J=9.4 Hz, 1H), 7.03-6.92 (m, 2H), 5.92 (s, 1H), 5.13-4.88 (m, 2H), 4.56-4.32 (m, 5H), 4.29 (s, 1H), 4.12 (s, 4H), 3.75-3.67 (m, 1H), 3.61 (d, J=9.8 Hz, 1H), 3.46 (dd, J=25.9, 11.4 Hz, 1H), 2.46 (s, 3H), 2.27-2.17 (m, 1H), 2.07-1.98 (m, 1H), 1.85-1.74 (m, 1H), 1.42 (dd, J=32.3, 7.0 Hz, 3H), 1.00-0.93 (m, 3H), 0.82 (dd, J=14.6, 6.6 Hz, 3H), LCMS (ESI) m/z: [M+H]⁺=800.20.

### Preparation of tert-butyl(S)-3-methyl-2-(4-(tributylstannyl)-1H-1,2,3-triazol-1-yl)butanoate

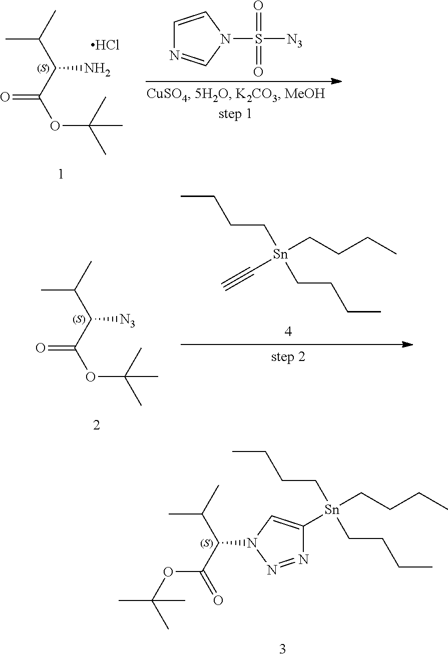

### Step 1: Preparation of tert-butyl tert-butyl(S)-2-azido-3-methylbutanoate (Intermediate 2)

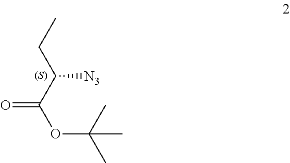

To a stirred mixture of tert-butyl L-valinate hydrochloride (5 g, 28.859 mmol, 1 equiv) and CuSO₄·5H₂O (1.44 g, 5.772 mmol, 0.1 equiv) in MeOH (80 mL) were added imidazole-1-sulfonyl azide (9.99 g, 57.718 mmol, 1 equiv) and K₂CO₃ (24.11 g, 173.154 mmol, 3 equiv) at room temperature. The resulting mixture was stirred for overnight at room temperature. The resulting mixture was diluted with water (300 mL) extracted with EtOAc (3×100 mL). The combined organic layers were washed with brine (2×200 mL), dried over anhydrous Na₂SO₄. After filtration, the filtrate was concentrated under reduced pressure. This resulted in Intermediate 2 (11.3 g, crude) as a light yellow oil. The crude product was used in the next step directly without further purification. LCMS (ESI) m/z: [M+H]⁺=200.

### Step 2: Preparation of tert-butyl (S)-3-methyl-2-(4-(tributylstannyl)-1H-1,2,3-triazol-1-yl)butanoate (Intermediate 3)

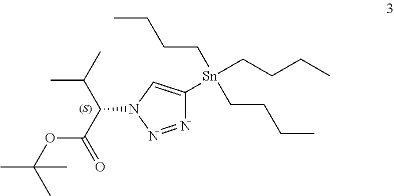

A mixture of Intermediate 2 (4 g, 20.075 mmol, 1 equiv) and tributyl(ethynyl)stannane (6.33 g, 20.075 mmol, 1 equiv) in toluene (20 mL) was stirred for overnight at 100° C. under nitrogen atmosphere. Desired product could be detected by LCMS. The residue was purified by silica gel column chromatography, eluted with CH₂Cl₂/PE (1:1) to afford Intermediate 3 (6.5 g, 62.95%) as a colorless liquid. LCMS (ESI) m/z [M+H]⁺=515.

### Preparation of Preparation of (2S,4R)-4-hydroxy-1-[(2S)-2-{4-[3-(2-hydroxyphenyl)pyrido[3,2-c]pyridazin-6-yl]-1,2,3-triazol-1-yl}-3-methylbutanoyl]-N-[(1S)-1-[4-(4-methyl-1,3-thiazol-5-yl)phenyl]ethyl]pyrrolidine-2-carboxamide (Compound 2)

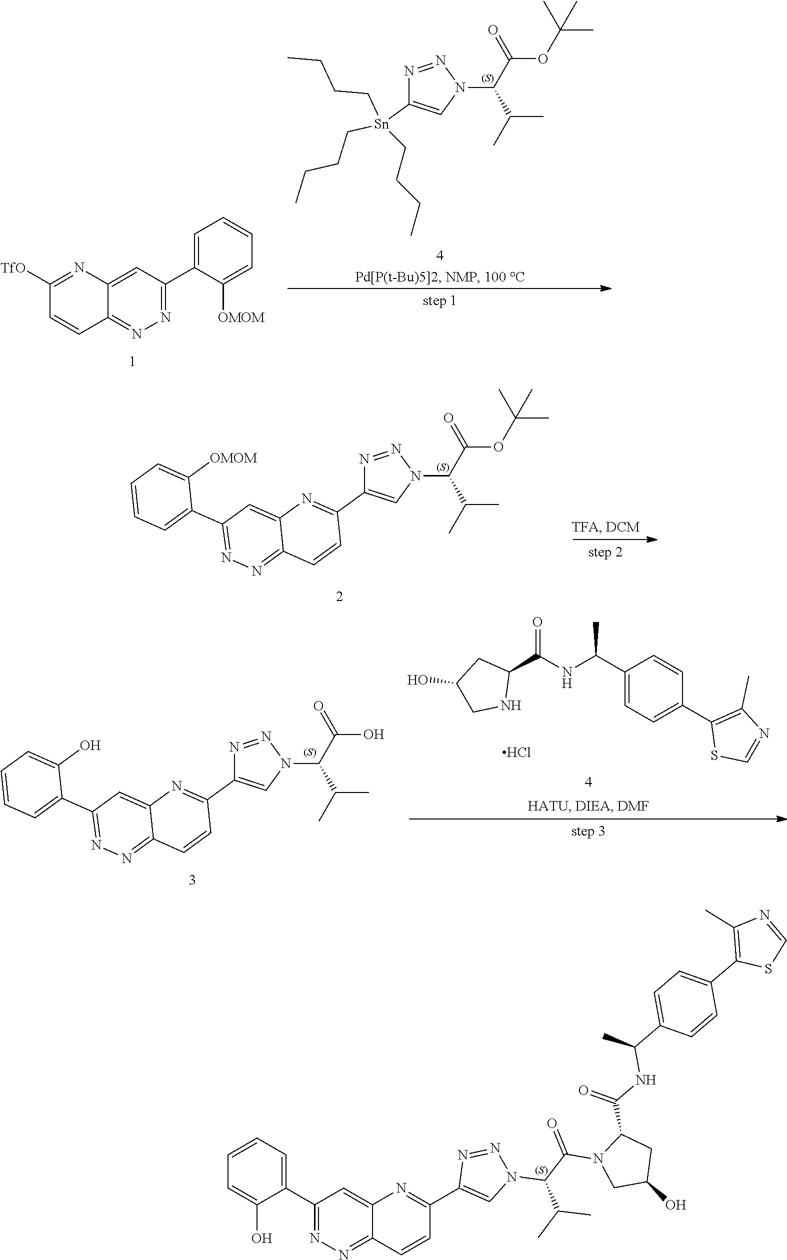

### Step 1: Preparation of tert-butyl(2S)-2-(4-{3-[2-(methoxymethoxy)phenyl]pyrido[3,2-c]pyridazin-6-yl}-1,2,3-triazol-1-yl)-3-methylbutanoate (Intermediate 2)

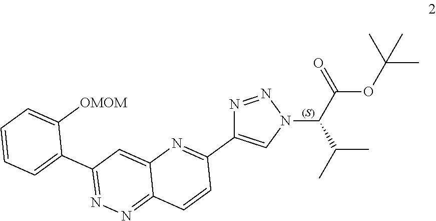

A mixture of 3-[2-(methoxymethoxy)phenyl]pyrido[3,2-c]pyridazin-6-yl trifluoromethanesulfonate (300 mg, 0.722 mmol, 1 equiv), tert-butyl (28)-3-methyl-2-[4-(tributylstannyl)-1,2,3-triazol-1-yl]butanoate (445.81 mg, 0.866 mmol, 1.2 equiv) and bis(tri-tert-butylphosphane) palladium (36.91 mg, 0.072 mmol, 0.1 equiv) in NMP (3 mL) was stirred for 1 h at 100° C. under nitrogen atmosphere. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 2 (85 mg, 23.99%) as a yellow solid. LCMS (ESI) m/z: [M+H]⁺=491.

### Step 2: Preparation of (28)-2-{4-[3-(2-hydroxyphenyl)pyrido[3,2-c]pyridazin-6-yl]-1,2,3-triazol-1-yl}-3-methylbutanoic acid (Intermediate 3)

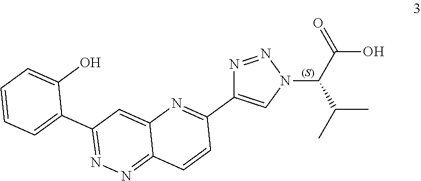

A mixture of Intermediate 2 (85 mg, 0.173 mmol, 1 equiv) and TFA (0.5 mL) in DCM (1.5 mL) was stirred for overnight at room temperature. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford Intermediate 3 (30 mg, 44.35%) as a yellow solid. LCMS (ESI) m/z [M+H]⁺=391.

### Step 3: Preparation of (2S,4R)-4-hydroxy-1-[(2S)-2-{4-[3-(2-hydroxyphenyl)pyrido[3,2-c]pyridazin-6-yl]-1,2,3-triazol-1-yl}-3-methylbutanoyl]-N-[(1S)-1-[4-(4-methyl-1,3-thiazol-5-yl)phenyl]ethyl]pyrrolidine-2-carboxamide (Compound 2)

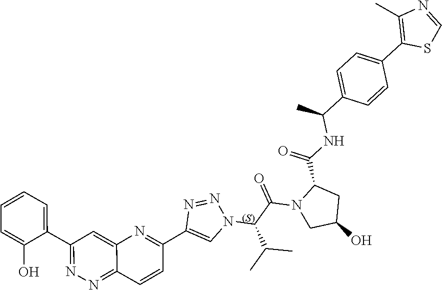

A mixture of Intermediate 3 (25 mg, 0.064 mmol, 1 equiv), (2S,4R)-4-hydroxy-N-[(1S)-1-[4-(4-methyl-1,3-thiazol-5-yl)phenyl]ethyl]pyrrolidine-2-carboxamide (31.84 mg, 0.096 mmol, 1.5 equiv), HATU (36.52 mg, 0,096 mmol, 1.5 equiv) and DIEA (24.83 mg, 0.192 mmol, 3 equiv) in DMF (1 mL) was stirred for 1 h at room temperature. Desired product could be detected by LCMS. The crude product was purified by C18 silica gel to afford Compound 2 (10.3 mg, 21.94%) as a yellow solid. ¹H NMR (400 MHz, DMSO-d6) δ 11.46 (s, 1H), 9.06 (s, 1H), 9.01-8.88 (m, 3H), 8.65-8.57 (m, 1H), 8.54 (d, J=7.6 Hz, 1H), 8.25 (dd, J=7.9, 1.7 Hz, 1H), 7.49-7.34 (m, 5H), 7.14-7.02 (m, 2H), 5.53 (d, J=10.0 Hz, 1H), 5.25-5.10 (m, 1H), 5.02-4.89 (m, 1H), 4.43 (t, J=8.1 Hz, 1H), 4.35 (s, 1H), 3.84-3.57 (m, 2H), 2.65-2.56 (m, 1H), 2.45 (s, 3H), 2.13-2.04 (m, 1H), 1.86-1.75 (m, 1H), 1.49 (dd, J=73.1, 7.0 Hz, 3H), 1.12 (d, J=6.5 Hz, 3H), 0.79 (d, J=6.6 Hz, 3H). LCMS (ESI) m/z: [M+H]⁺=704.20.

### Preparation of (2S,4R)-4-hydroxy-1-[(2R)-2-(3-{6-[3-(2-hydroxyphenyl)-5-methyl-6-oxopyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 4)

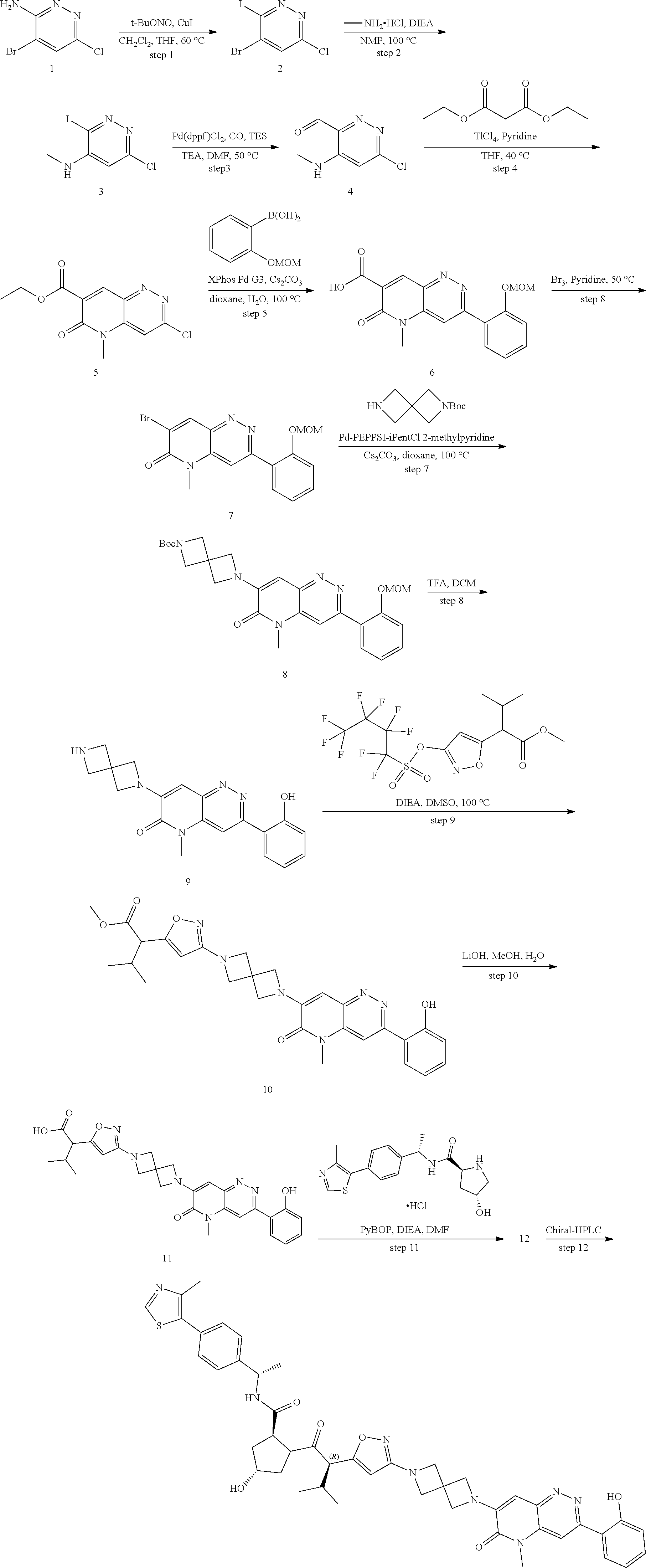

### Step 1: Preparation of 4-bromo-6-chloro-3-iodopyridazine (Intermediate 2)

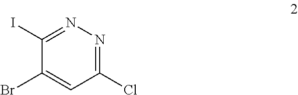

To a stirred solution of 4-bromo-6-chloropyridazin-3-amine (20 g, 95.951 mmol, 1 equiv) and CuI (21.93 g, 115.141 mmol, 1.2 equiv) in THE (60 mL) were added CH₂I₂ (30.84 g, 115.141 mmol, 1.2 equiv) and t-BuNO₂ (9.89 g, 95.951 mmol, 1 equiv). The resulting solution was stirred at 60 degrees C. for 5 hours. The reaction mixture was diluted with EtOAc (500 mL) and washed with water (3×500 mL). The organic layer was dried over anhydrous Na₂SO₄. After filtration, the filtrate was concentrated under reduced pressure. The residue was purified by silica gel column chromatography, elution gradient 0 to 80% EtOAc in petroleum ether to give intermediate 2 (7.5 g, 24.41%) as a yellow solid. LCMS (ESI) m/z: [M+H]⁺=319.

### Step 2: Preparation of 6-chloro-3-iodo-N-methylpyridazin-4-amine (Intermediate 3)

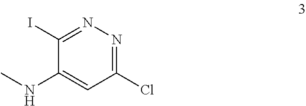

To a solution of intermediate 2 (7.5 g, 23.487 mmol, 1 equiv) and methanamine, hydrochloride (1.90 g, 28.184 mmol, 1.2 equiv) in NMP (50 mL) was added DIEA (6.07 g, 46.974 mmol, 2 equiv). The resulting solution was stirred at 80 degrees C. for 2 hours. The resulting mixture was diluted with ethyl acetate (300 mL) and washed with water (3×300 mL). The organic layer was dried over anhydrous sodium sulfate, filtered and concentrated to get the residue. The residue was purified by silica gel column chromatography, elution gradient 0 to 55% EtOAc in petroleum ether to afford intermediate 3 (4.15 g, 65.57%) as a white solid. LCMS (ESI) m/z: [M+H]⁺=270.

### Step 3: Preparation of 6-chloro-4-(methylamino)pyridazine-3-carbaldehyde (Intermediate 4)

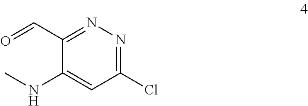

To a solution of intermediate 3 (4.15 g, 15.401 mmol, 1 equiv) and Pd(dppf)Cl₂ (1.13 g, 1.540 mmol, 0.1 equiv) in DMF (30 mL) were added Triethylsilane (5.37 g, 46.202 mmol, 3 equiv) and TEA (4.68 g, 46.202 mmol, 3 equiv). The mixture was pressurized to 40 atm with carbon monoxide. The resulting solution was stirred at 40 degrees C. for 3 hours. The reaction solution was purified by flash C18 chromatography, elution gradient 0 to 50% acetonitrile in water to afford intermediate 4 (997 mg, 30.11%) as a brown solid. LCMS (ESI) m/z: [M+H]⁺=172.

### Step 4: Preparation of ethyl 3-chloro-5-methyl-6-oxopyrido[3,2-c]pyridazine-7-carboxylate (Intermediate 5)

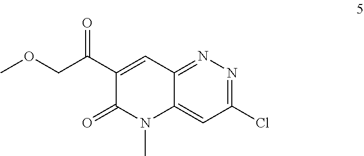 [SMILES: COCC(=O)c1cc2nnc(Cl)cc2n(C)c1=O]

To a solution of intermediate 4 (0.993 g, 5.787 mmol, 1 equiv) and diethyl malonate (1.11 g, 6.944 mmol, 1.2 equiv) in THF (10.00 mL, 123.437 mmol, 21.33 equiv) were added TiCl₄ (2.20 g, 11.574 mmol, 2 equiv) and pyridine (0.92 g, 11.574 mmol, 2 equiv) at 0 degrees C. The resulting solution was stirred at 40 degrees C. for 3 hours. The resulting mixture was diluted with ethyl acetate (200 mL) and washed with water (3×200 mL). The organic layer was dried over anhydrous sodium sulfate, filtered and concentrated to get the residue. The residue was purified by silica gel column chromatography, elution gradient 0 to 55% ethyl acetate in petroleum ether to afford intermediate 5 (1.01 g, 65.20%) as an off-white solid. LCMS (ESI) m/z: [M+H]⁺=268.

### Step 5: Preparation of 3-[2-(methoxymethoxy)phenyl]-5-methyl-6-oxopyrido[3,2-c]pyridazine-7-carboxylic acid (Intermediate 6)

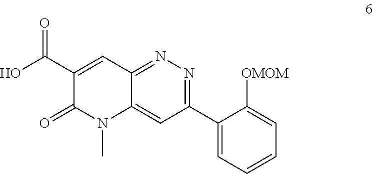

To a solution of intermediate 5 (1 g, 3.736 mmol, 1 equiv) and 2-(methoxymethoxy)phenylboronic acid (1.02 g, 5.604 mmol, 1.5 equiv) in dioxane (8 mL, 94.432 mmol, 25.28 equiv) and H₂O (2 mL, 111.019 mmol, 29.72 equiv) were added XPhos Pd G3 (0.32 g, 0.374 mmol, 0.1 equiv) and Cs₂CO₃ (2.43 g, 7.472 mmol, 2 equiv). The resulting solution was stirred at 100 degrees C. for 5 hours under N₂ atmosphere. The resulting mixture was diluted with ethyl acetate (200 mL) and washed with water (3×200 mL). The organic layer was dried over anhydrous sodium sulfate, filtered and concentrated to get the residue. The residue was purified by flash C18 chromatography, elution gradient 0 to 72% acetonitrile in water to afford intermediate 6 (890 mg, 69.79%) as a yellow solid. LCMS (ESI) m/z: [M+H]⁺=342.

### Step 6: Preparation of 7-bromo-3-[2-(methoxymethoxy)phenyl]-5-methylpyrido[3,2-c]pyridazin-6-one (Intermediate 7)

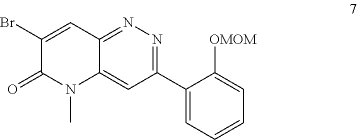

To a solution of intermediate 6 (890 mg, 2.608 mmol, 1 equiv) in pyridine (10 mL) was added Br₂ (833.40 mg, 5.216 mmol, 2 equiv). The resulting solution was stirred at 60 degrees C. for 2 hours. The resulting mixture was diluted with ethyl acetate (200 mL) and washed with water (3×200 mL). The organic layer was dried over anhydrous sodium sulfate, filtered and concentrated to get the residue. The residue was purified by silica gel column chromatography, elution gradient 0 to 45% ethyl acetate in petroleum ether to afford intermediate 7 (930 mg, 94.80%) as a yellow solid. LCMS (ESI) m/z: [M+H]⁺=376.

### Step 7: Preparation of tert-butyl 6-{3-[2-(methoxymethoxy)phenyl]-5-methyl-6-oxopyrido[3,2-c]pyridazin-7-yl}-2,6-diazaspiro[3.3]heptane-2-carboxylate (Intermediate 8)

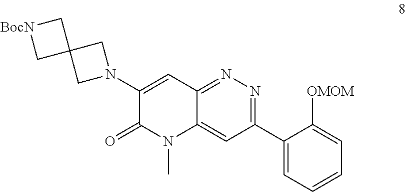

To a solution of intermediate 7 (700 mg, 1.861 mmol, 1 equiv) and tert-butyl 2,6-diazaspiro[3.3]heptane-2-carboxylate (553.36 mg, 2.792 mmol, 1.5 equiv) in dioxane (10 mL) were added Pd-PEPPSI-IPentCl 2-methylpyridine (o-picoline) (156.51 mg, 0.186 mmol, 0.1 equiv) and Cs₂CO₃ (1212.48 mg, 3.722 mmol, 2 equiv). The resulting solution was stirred at 100 degrees C. for 2 hours under N₂ atmosphere. The resulting mixture was diluted with ethyl acetate (200 mL) and washed with water (3×200 mL). The organic layer was dried over anhydrous sodium sulfate, filtered and concentrated to get the residue. The residue was purified by silica gel column chromatography, elution gradient 0 to 52% ethyl acetate in petroleum ether to afford intermediate 8 (705 mg, 76.77%) as a yellow solid. LCMS (ESI) m/z: [M+H]⁺=494.

### Step 8: Preparation of 7-{2,6-diazaspiro[3.3]heptan-2-yl}-3-(2-hydroxyphenyl)-5-methylpyrido[3,2-c]pyridazin-6-one (Intermediate 9)

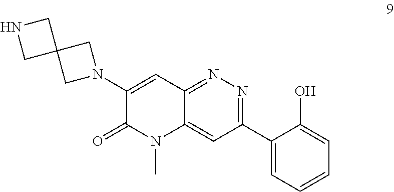 [SMILES: Cn1c(=O)c(N2CC3(CNC3)C2)cc2nnc(-c3ccccc3O)cc21]

To a solution of intermediate 8 (705 mg, 1.428 mmol, 1 equiv) in DCM (6 mL) was added TFA (2 mL). The resulting solution was stirred at room temperature for 2 hours. The resulting mixture was concentrated under reduced pressure to give intermediate 9 (647 mg, crude) as a yellow solid. LCMS (ESI) m/z: [M+H]⁺=350.

### Step 9: Preparation of methyl 2-(3-{6-[3-(2-hydroxyphenyl)-5-methyl-6-oxopyrido[3,2-c]pyridazin-7-yl]-2,6-diazaspiro[3.3]heptan-2-yl}-1,2-oxazol-5-yl)-3-methylbutanoate (Intermediate 10)

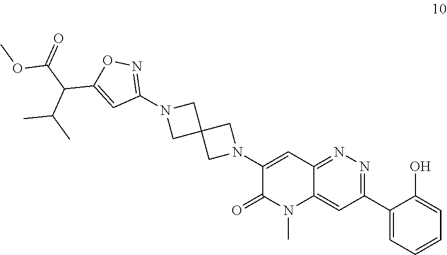 [SMILES: COC(=O)C(c1cc(N2CC3(C2)CN(c2cc4nnc(-c5ccccc5O)cc4n(C)c2=O)C3)no1)C(C)C]

To a solution of intermediate 9 (647 mg, 1.852 mmol, 1 equiv) and methyl 3-methyl-2-{3-[(1,1,2,2,3,3,4,4,4-nonafluorobutanesulfonyl)oxy]-1,2-oxazol-5-yl}butanoate (1336.86 mg, 2.778 mmol, 1.5 equiv) in DMSO (10 mL) was added DIEA (718.01 mg, 5.556 mmol, 3 equiv). The resulting solution was stirred at 100 degrees C. for 5 hours. The reaction mixture was purified by flash C18 chromatography, elution gradient 0 to 63% acetonitrile in water to afford intermediate 10 (420 mg, 42.75%) as a brown solid. LCMS (ESI) m/z: [M+H]⁺=531.

### Step 10: Preparation of 2-(3-{6-[3-(2-hydroxyphenyl)-5-methyl-6-oxopyrido[3,2-c]pyridazin-7-yl]-2,6-diazaspiro[3.3]heptan-2-yl}-1,2-oxazol-5-yl)-3-methylbutanoic acid (Intermediate 11)

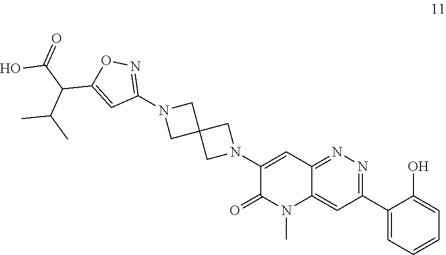 [SMILES: CC(C)C(C(=O)O)c1cc(N2CC3(C2)CN(c2cc4nnc(-c5ccccc5O)cc4n(C)c2=O)C3)no1]

To a solution of intermediate 10 (420 mg, 0.322 mmol, 1 equiv) in H₂O (1 mL) and MeOH (4 mL) was added LiOH (94.79 mg, 3.957 mmol, 5 equiv). The resulting solution was stirred at room temperature for 2 hours. The reaction solution was acidified to pH 5 with 1 M HCl (aq). The mixture was extracted with EtOAc (3×100 mL) and dried over anhydrous sodium sulfate, filtered and concentrated to give intermediate 11 (345 mg, 84.37%) as a yellow solid. LCMS (ESI) m/z: [M+H]⁺=517.

### Step 11: Preparation of (2S,4R)-4-hydroxy-1-[(2)-2-(3-{6-[3-(2-hydroxyphenyl)-5-methyl-6-oxopyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Intermediate 12)

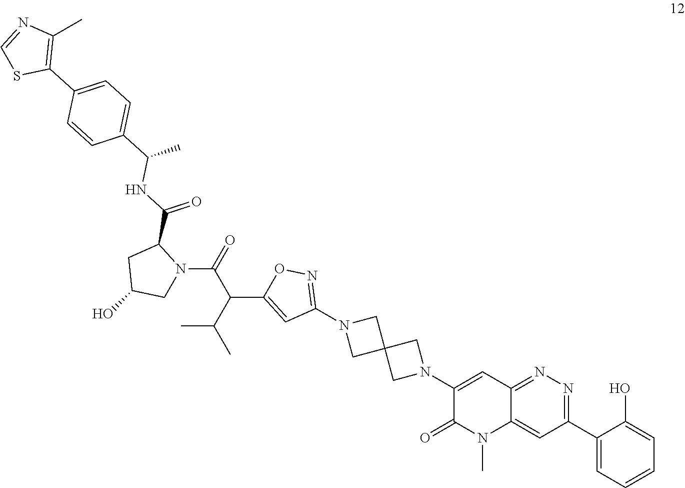 [SMILES: Cc1ncsc1-c1ccc([C@H](C)NC(=O)[C@@H]2C[C@@H](O)CN2C(=O)C(c2cc(N3CC4(C3)CN(c3cc5nnc(-c6ccccc6O)cc5n(C)c3=O)C4)no2)C(C)C)cc1]

To a solution of intermediate 11 (345 mg, 0.668 mmol, 1 equiv) and (2S,4R)-4-hydroxy-N-[(1S)-1-[4-(4-methyl-1,3-thiazol-5-yl)phenyl]ethyl]pyrrolidine-2-carboxamide (332.03 mg, 1.002 mmol, 1.5 equiv) in DMF (3 mL) were added PyBOP (695.13 mg, 1.336 mmol, 2 equiv) and DIEA (431.61 mg, 3.340 mmol, 5 equiv). The resulting solution was stirred at room temperature for 2 hours. The resulting solution was purified by Prep-HPLC with the following conditions: Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min mL/min; Gradient: 37% B to 52% B in 10 min; Wave Length: 254 nm/220 nm nm; RT1(min): 9.57 to give intermediate 12 (92 mg, 16.60%) as a yellow solid. LCMS (ESI) m/z: [M+H]⁺=830.

### Step 12: Preparation of (2S,4R)-4-hydroxy-1-[(2R)-2-(3-{6-[3-(2-hydroxyphenyl)-5-methyl-6-oxopyrido[3,2-c]pyridazin-7-yl]-2,6-diazaspiro[3.3]heptan-2-yl}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 4)

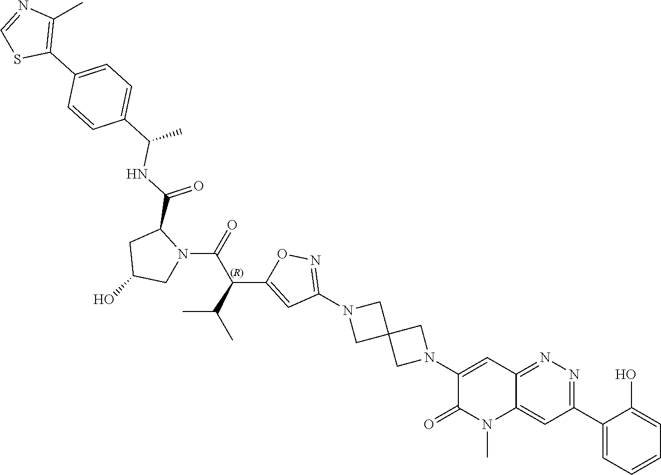

The intermediate 12 (92 mg) was purified by SFC with the following conditions: Column: CHIRAL ART Cellulose-SB, 3*25 cm, 5 μm; Mobile Phase A: CO₂, Mobile Phase B: MeOH:DCM=1:1 (20 mMNH₃); Flow rate: 100 mL/min; Gradient: isocratic 55% B; Column Temperature (° C.): 35; Back Pressure (bar): 100; Wave Length: 260/240 nm; RT1(min): 3; RT2(min): 5.17; Sample Solvent: HFIP; Injection Volume: 4 mL. Compound 4 (second peak) (44 mg, 47.83%) was obtained as a yellow solid. ¹H NMR (400 MHz, DMSO-d6) δ 13.57 (d, J=3.4 Hz, 1H), 8.99 (d, J=4.7 Hz, 1H), 8.41 (d, J=7.7 Hz, 1H), 8.21-8.14 (m, 1H), 8.08 (s, 1H), 7.51-7.41 (m, 2H), 7.41-7.31 (m, 3H), 7.00 (ddd, J=8.2, 6.5, 1.3 Hz, 2H), 6.65 (s, 1H), 5.90 (s, 1H), 5.11 (d, J=3.6 Hz, 1H), 4.91 (q, J=6.9 Hz, 1H), 4.61-4.15 (s, 6H), 4.08 (s, 4H), 3.76-3.65 (m, 4H), 3.63-3.54 (m, 1H), 3.45 (dd, J=16.1, 9.9 Hz, 1H), 2.46 (d, J=4.4 Hz, 3H), 2.28-2.15 (m, 1H), 2.03 (t, J=8.8 Hz, 1H), 1.79 (ddd, J=12.8, 8.0, 4.8 Hz, 1H), 1.46-1.24 (m, 3H), 0.96 (t, J=6.5 Hz, 3H), 0.82 (dd, J=19.3, 6.9 Hz, 3H). LCMS (ESI) m/z: [M+H]⁺=830.30.

### Example 2. Degradation of BRM and BRG1 by Compounds of the Invention

This example demonstrates the ability of the compounds of the disclosure to degrade a HiBit-BRM or HiBit-BRG1 fusion protein in a cell-based degradation assay.

Procedure: A stable HeLa cell line expressing HiBiT-BRM was generated. On day 0, 5000 cells were seeded in 40 μL of media into each well of 384-well cell culture plates. On day 1, cells were treated with 120 nL DMSO or 120 nL of 3-fold serially DMSO-diluted compounds (10 points in duplicate with 30 UM as final top dose). Subsequently plates were incubated for 24 h in a standard tissue culture incubator and equilibrated at room temperature for 15 minutes. Nano-Glo HiBiT Lytic Detection System (Promega N3050) reagent was freshly prepared and 20 μl was added to each well. Upon addition of this LgBit-containing reagent, the HiBIT and LgBiT proteins associate to form the luminescent NanoBiT luciferase. The plates were shaken for 10 minutes at room temperature and the bioluminescence read using an En Vision plate reader (PerkinElmer).

For measurement of BRG1 degradation, a stable HeLa cell line expressing HiBit-BRG1 and LgBit was generated. The same protocol as above was then followed.

The degradation % was calculated using the following formula: % degradation=100%−100%×(Lumₛₐₘₚₗₑ−Lum_{LC})/(Lum_{HC}−Lum_{LC}). DMSO treated cells are employed as High Control (HC) and 2 μM of a known BRM/BRG1 degrader standard treated cells are employed as Low Control (LC). The data was fit to a four parameter, non-linear curve fit to calculate IC₅₀ (μM) values as shown in Table 2.

Results: As shown in Table 2 below, the compounds of the invention degraded BRM and/or BRG1.

TABLE 2

| | BRM HiBit | BRM | BRMG1 | BRG1 HiBit |
|---|---|---|---|---|
| | Degradation | HiBit | HiBit | Degradation |
| Compound | DC50 | Degradation | Degradation | Maximum |
| No. | (nM) | Maximum | DC50 (nM) | (%) |
| 1 | ++++ | A | NC | C |
| 2 | ++++ | A | ++++ | A |
| 3 | ++++ | A | NC | C |
| 4 | ++++ | A | NC | C |

“+” indicates DC50 ≥ 1000 nM;
“++” indicates DC50 of < 1000 nM and ≥ 100 nM;
“+++” indicates DC50 of < 100 nM and ≥ 10 nM;
“++++” indicates inhibitory effect of < 10 nM;
“NT” indicates not tested;
“NC” indicates not calculated;
“A” indicates maximum degradation ≥ 75%;
“B” indicates maximum degradation ≥ 50%; and
“C” indicates maximum degradation < 50%.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1.-227. (canceled)

228. A compound, or a pharmaceutically acceptable salt thereof, of Formula I: 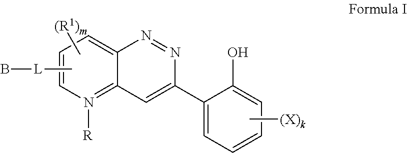
wherein
m is 0, 1, 2, or 3;
k is 0, 1, or 2;
R is absent or optionally substituted C₁-C₆ alkyl;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl, optionally substituted C₃-C₈ cycloalkoxy, optionally substituted C₂-C₆ alkynyl, optionally substituted amino, or cyano;
each X is, independently, halo or optionally substituted C₁-C₆ heteroalkyl;
L is a linker of Formula II: 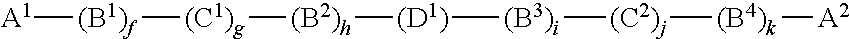
or a pharmaceutically acceptable salt thereof,
wherein
A¹ is a bond between the linker and the ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₃-C₁₀ carbocyclyl, optionally substituted C₂-C₁₀ heterocyclyl, optionally substituted C₂-C₆ heteroaryl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, optionally substituted C₃-C₁₀ cycloalkyl, optionally substituted C₃-C₁₀ carbocyclyl, or optionally substituted C₁₋₁₀ heteroalkyl; or D is absent, and the linker is A¹-(B¹)_{f}—(C¹)_{g}—(B²)ₕ—(B³)ᵢ—(C²)ⱼ—(B⁴)ₖ-A²; and
B is a degradation moiety of Formula A-1: 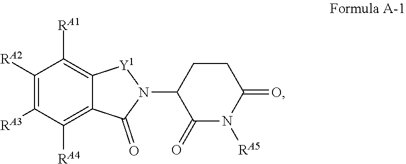
wherein
Y¹ is 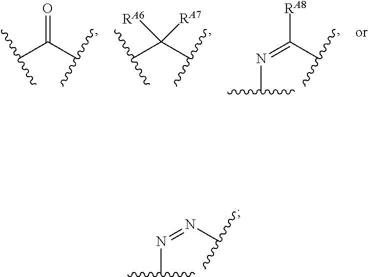
R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form 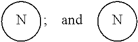
and is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or 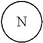
is substituted with A²; and
A² is a bond between the degradation moiety and the linker; or
a degradation moiety of Formula C: 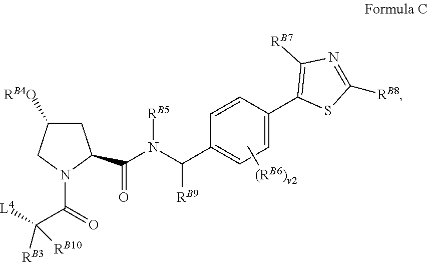
wherein
L⁴ is —N(R^{B1})(R^{B2}), 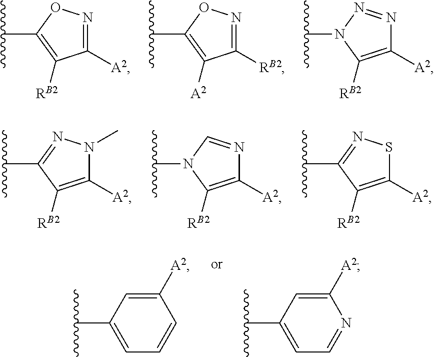
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B10} is H or F; and
A² is a bond between the degradation moiety and the linker;
wherein one and only one of R^{B1}, R^{B3}, and R^{B6} is A²,
or a pharmaceutically acceptable salt thereof; or
a degradation moiety of Formula D: 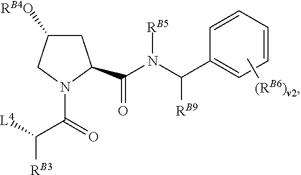
where
L⁴ is —N(R^{B1})(R^{B2}), 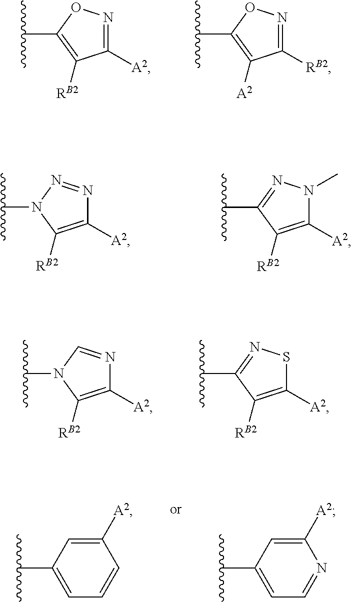
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₂-C₆ alkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof; or
a degradation moiety of Formula E: 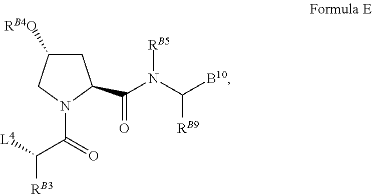
where
L⁴ is —N(R^{B1})(R^{B2}), 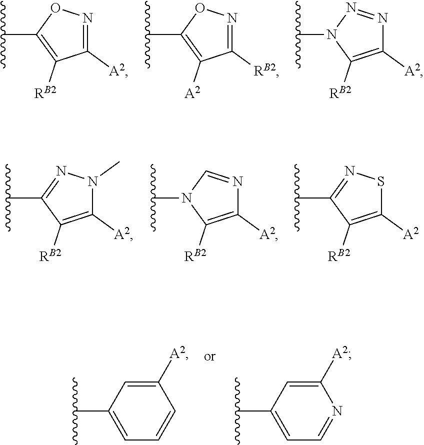
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
R^{B9} is H, optionally substituted C₁-C₆ alkyl, optionally substituted C₃-C₆ alkynyl, optionally substituted C₃-C₁₀ carbocyclyl, or optionally substituted C₂-C₁₀ heterocyclyl;
B¹⁰ is H, optionally substituted C₁-C₆ alkyl, optionally substituted C₃-C₆ alkynyl, optionally substituted C₃-C₁₀ carbocyclyl, optionally substituted C₂-C₁₀ heterocyclyl, optionally substituted amino, or cyano, and
A² is a bond between the degradation moiety and the linker;
where one and only one of R^{B1}, R^{B3}, and R^{B6} is A²,
or a pharmaceutically acceptable salt thereof; or
a degradation moiety of Formula F: 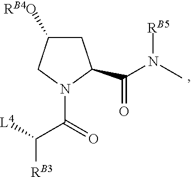
where
L⁴ is —N(R^{B1})(R^{B2}), 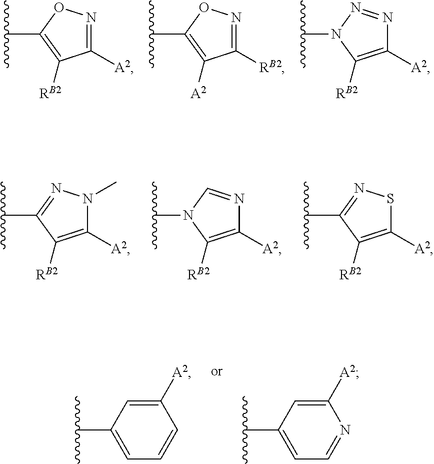
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
A² is a bond between the degradation moiety and the linker;
where one and only one of R^{B1} or R^{B3} is A²,
or a pharmaceutically acceptable salt thereof.

229. The compound of claim 228, or a pharmaceutically acceptable salt thereof, wherein the compound has the structure of Formula I-A or I-B:

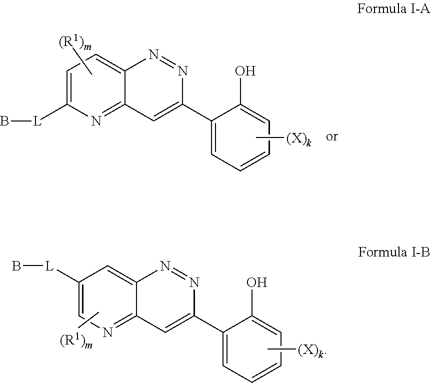

230. The compound of claim 228, or a pharmaceutically acceptable salt thereof, wherein the compound has the structure of Formula I-C:

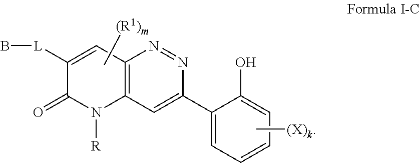

231. The compound of claim 228, or a pharmaceutically acceptable salt thereof, wherein m is 0 or 1.

232. The compound of claim 228, or a pharmaceutically acceptable salt thereof, wherein R¹ is C₁-C₆ alkoxy or halo.

233. The compound of claim 232, or a pharmaceutically acceptable salt thereof, wherein R¹ is methoxy or difluoromethoxy.

234. The compound of claim 232, or a pharmaceutically acceptable salt thereof, wherein R¹ is F or Cl.

235. The compound of claim 228, or a pharmaceutically acceptable salt thereof, wherein R¹ is optionally substituted C₁-C₆ alkyl.

236. The compound of claim 235, or a pharmaceutically acceptable salt thereof, wherein R¹ is methyl or difluoromethyl.

237. The compound of claim 228, or a pharmaceutically acceptable salt thereof, wherein R¹ is optionally substituted C₂-C₆ alkynyl,

238. The compound of claim 237, or a pharmaceutically acceptable salt thereof, wherein R¹ is methyne.

239. The compound of claim 228, or a pharmaceutically acceptable salt thereof, wherein R¹ is optionally substituted amino, or cyano.

240. The compound of claim 239, or a pharmaceutically acceptable salt thereof, wherein k is 0 or 1.

241. The compound of claim 239, or a pharmaceutically acceptable salt thereof, wherein X is optionally substituted C₁-C₆ heteroalkyl or halo.

242. The compound of claim 241, or a pharmaceutically acceptable salt thereof, wherein X is methoxy or F.

243. The compound of claim 242, or a pharmaceutically acceptable salt thereof, wherein the degradation moiety has the structure of

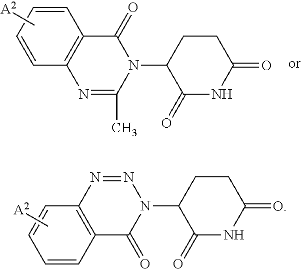

244. The compound of claim 242, or a pharmaceutically acceptable salt thereof, wherein the degradation moiety is

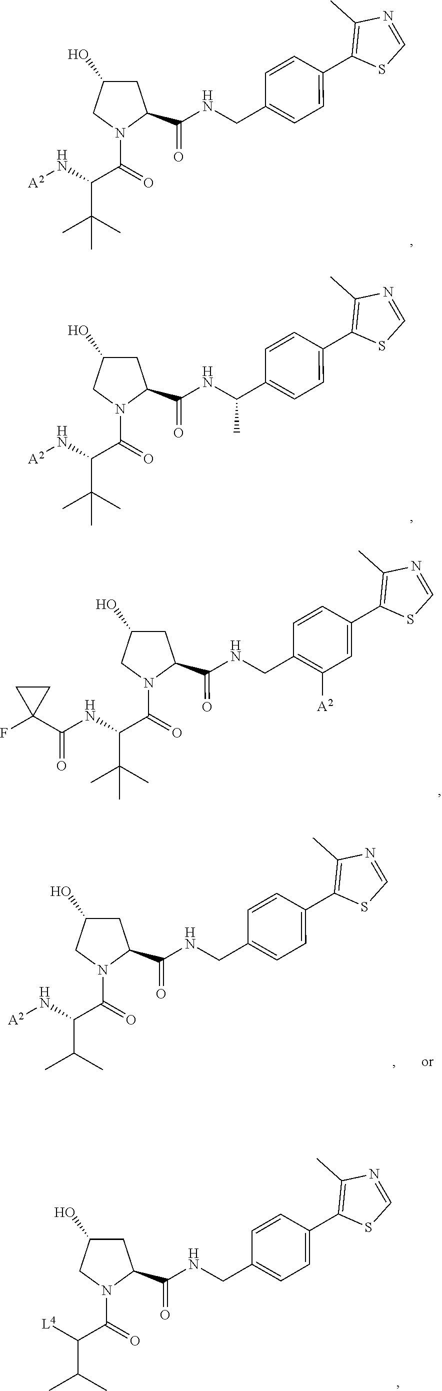

245. The compound of claim 242, or a pharmaceutically acceptable salt thereof, wherein the degradation moiety is

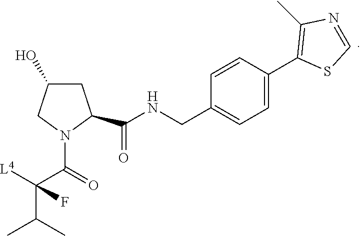

246. The compound of claim 242, or a pharmaceutically acceptable salt thereof, wherein the degradation moiety is

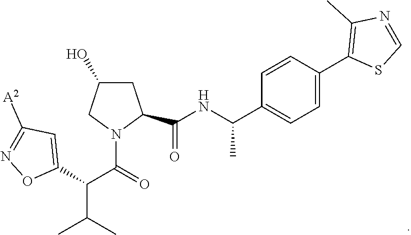

247. The compound of claim 242, or a pharmaceutically acceptable salt thereof, wherein the degradation moiety is

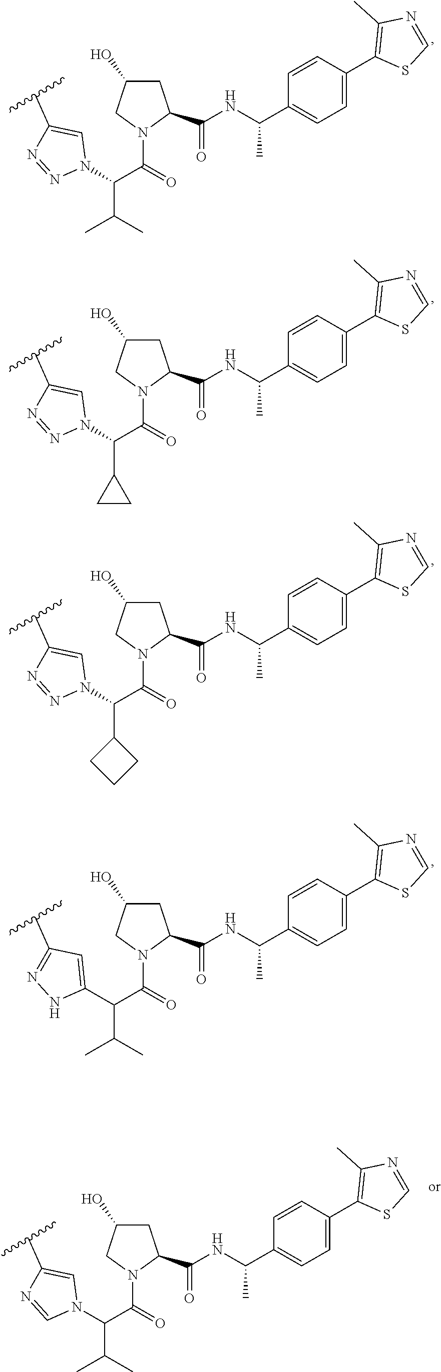 [SMILES: *c1cn([C@H](C(=O)N2C[C@H](O)C[C@H]2C(=O)N[C@@H](C)c2ccc(-c3scnc3C)cc2)C(C)C)nn1 ; *c1cn([C@H](C(=O)N2C[C@H](O)C[C@H]2C(=O)N[C@@H](C)c2ccc(-c3scnc3C)cc2)C2CC2)nn1 ; *c1cn([C@H](C(=O)N2C[C@H](O)C[C@H]2C(=O)N[C@@H](C)c2ccc(-c3scnc3C)cc2)C2CCC2)nn1 ; *c1cc(C(C(=O)N2C[C@H](O)C[C@H]2C(=O)N[C@@H](C)c2ccc(-c3scnc3C)cc2)C(C)C)[nH]n1 ; *c1cn(C(C(=O)N2C[C@H](O)C[C@H]2C(=O)N[C@@H](C)c2ccc(-c3scnc3C)cc2)C(C)C)cn1]

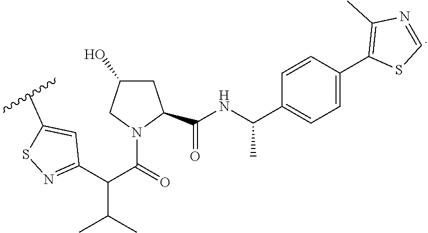 [SMILES: *c1cc(C(C(=O)N2C[C@H](O)C[C@H]2C(=O)N[C@@H](C)c2ccc(-c3scnc3C)cc2)C(C)C)ns1]

248. The compound of claim 242, or a pharmaceutically acceptable salt thereof, wherein the degradation moiety is

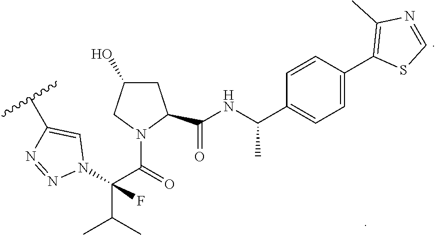

249. The compound of claim 242, or a pharmaceutically acceptable salt thereof, wherein the degradation moiety is

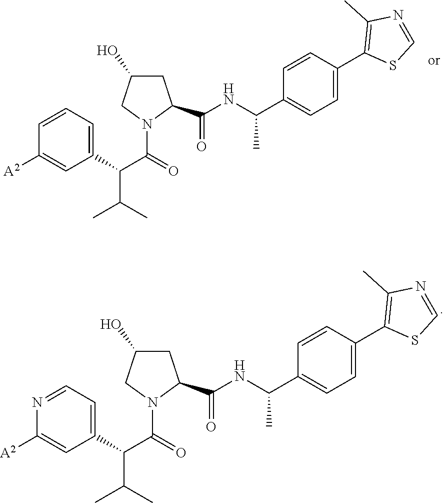

250. The compound of claim 242, or a pharmaceutically acceptable salt thereof, wherein the degradation moiety is

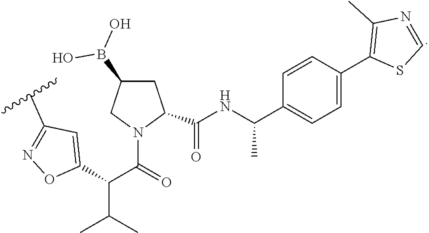 [SMILES: *c1cc([C@H](C(=O)N2C[C@@H](B(O)O)C[C@@H]2C(=O)N[C@@H](C)c2ccc(-c3scnc3C)cc2)C(C)C)on1]

251. The compound of claim 242, or a pharmaceutically acceptable salt thereof, wherein the degradation moiety is

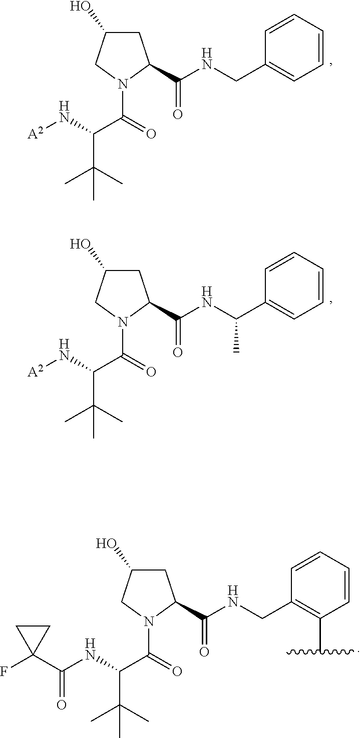

252. The compound of claim 242, or a pharmaceutically acceptable salt thereof, wherein the degradation moiety is

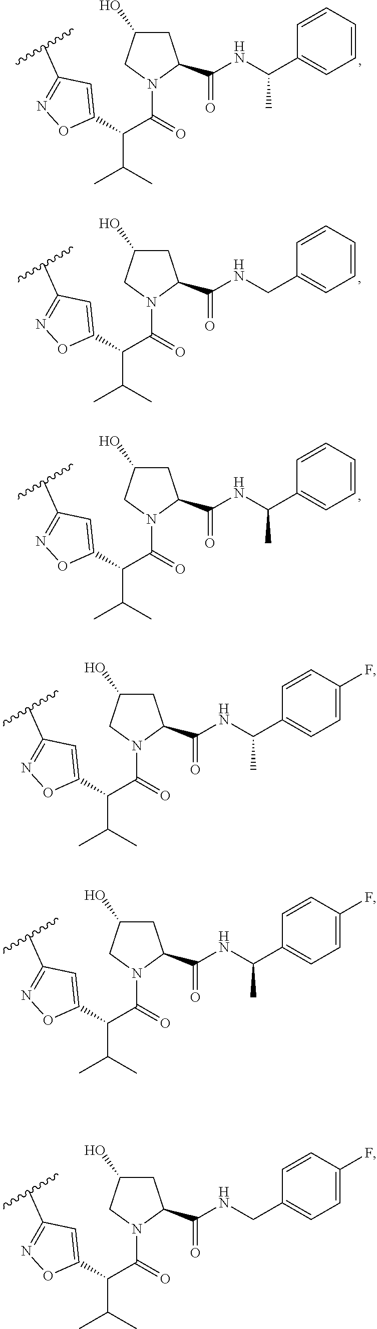

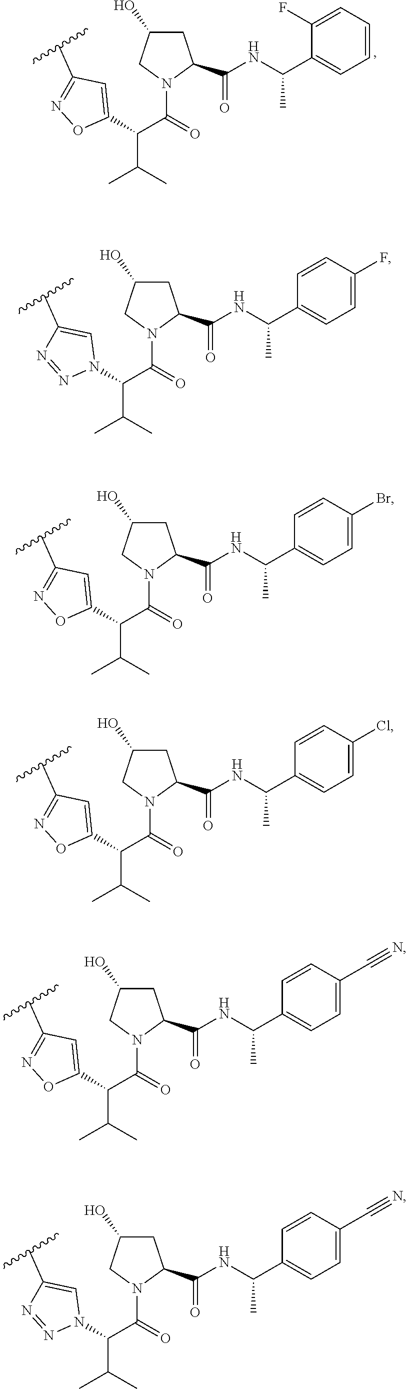 [SMILES: *c1cc([C@H](C(=O)N2C[C@H](O)C[C@H]2C(=O)N[C@@H](C)c2ccccc2F)C(C)C)on1 ; *c1cn([C@H](C(=O)N2C[C@H](O)C[C@H]2C(=O)N[C@@H](C)c2ccc(F)cc2)C(C)C)nn1 ; *c1cc([C@H](C(=O)N2C[C@H](O)C[C@H]2C(=O)N[C@@H](C)c2ccc(Br)cc2)C(C)C)on1 ; *c1cc([C@H](C(=O)N2C[C@H](O)C[C@H]2C(=O)N[C@@H](C)c2ccc(Cl)cc2)C(C)C)on1 ; *c1cc([C@H](C(=O)N2C[C@H](O)C[C@H]2C(=O)N[C@@H](C)c2ccc(C#N)cc2)C(C)C)on1 ; *c1cn([C@H](C(=O)N2C[C@H](O)C[C@H]2C(=O)N[C@@H](C)c2ccc(C#N)cc2)C(C)C)nn1]

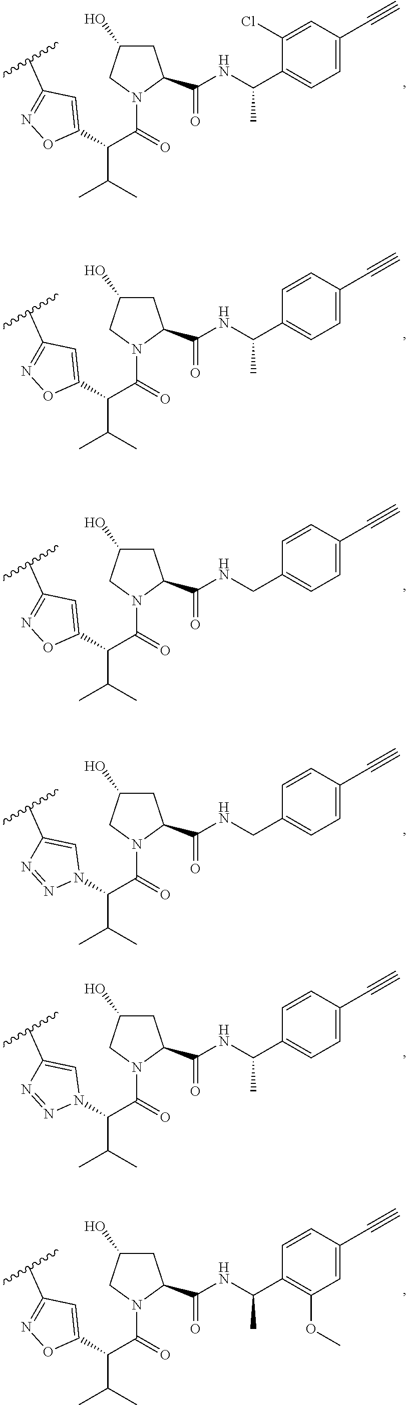 [SMILES: *c1cc([C@H](C(=O)N2C[C@H](O)C[C@H]2C(=O)N[C@@H](C)c2ccc(C#C)cc2Cl)C(C)C)on1 ; *c1cc([C@H](C(=O)N2C[C@H](O)C[C@H]2C(=O)N[C@@H](C)c2ccc(C#C)cc2)C(C)C)on1 ; *c1cc([C@H](C(=O)N2C[C@H](O)C[C@H]2C(=O)NCc2ccc(C#C)cc2)C(C)C)on1 ; *c1cn([C@H](C(=O)N2C[C@H](O)C[C@H]2C(=O)NCc2ccc(C#C)cc2)C(C)C)nn1 ; *c1cn([C@H](C(=O)N2C[C@H](O)C[C@H]2C(=O)N[C@@H](C)c2ccc(C#C)cc2)C(C)C)nn1 ; *c1cc([C@H](C(=O)N2C[C@H](O)C[C@H]2C(=O)N[C@H](C)c2ccc(C#C)cc2OC)C(C)C)on1]

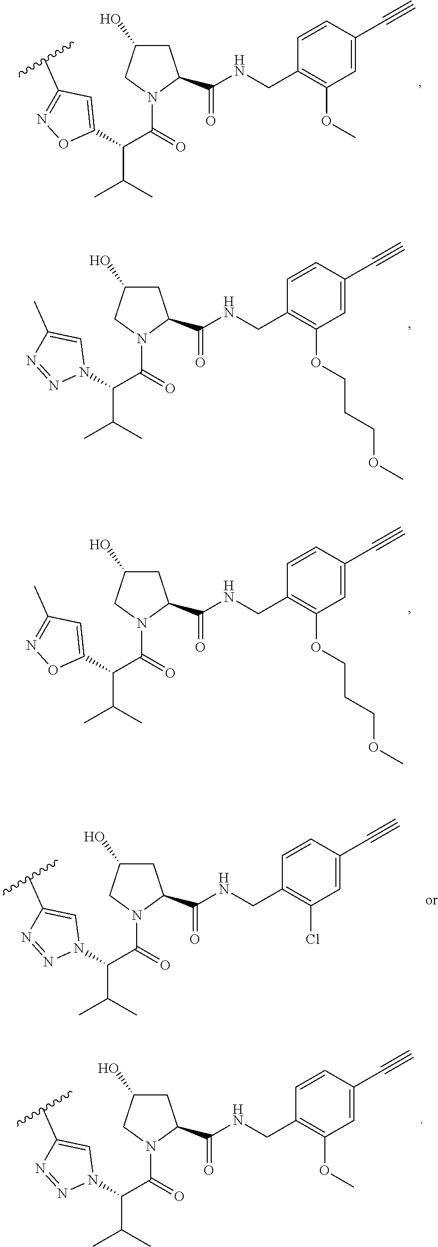 [SMILES: *c1cc([C@H](C(=O)N2C[C@H](O)C[C@H]2C(=O)NCc2ccc(C#C)cc2OC)C(C)C)on1 ; C#Cc1ccc(CNC(=O)[C@@H]2C[C@@H](O)CN2C(=O)[C@H](C(C)C)n2cc(C)nn2)c(OCCCOC)c1 ; C#Cc1ccc(CNC(=O)[C@@H]2C[C@@H](O)CN2C(=O)[C@@H](c2cc(C)no2)C(C)C)c(OCCCOC)c1 ; *c1cn([C@H](C(=O)N2C[C@H](O)C[C@H]2C(=O)NCc2ccc(C#C)cc2Cl)C(C)C)nn1 ; *c1cn([C@H](C(=O)N2C[C@H](O)C[C@H]2C(=O)NCc2ccc(C#C)cc2OC)C(C)C)nn1]

253. The compound of claim 242, or a pharmaceutically acceptable salt thereof, wherein the degradation moiety is

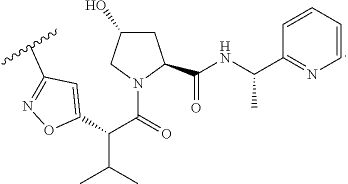 [SMILES: *c1cc([C@H](C(=O)N2C[C@H](O)C[C@H]2C(=O)N[C@@H](C)c2ccccn2)C(C)C)on1]

254. The compound of claim 242, or a pharmaceutically acceptable salt thereof, wherein the degradation moiety is

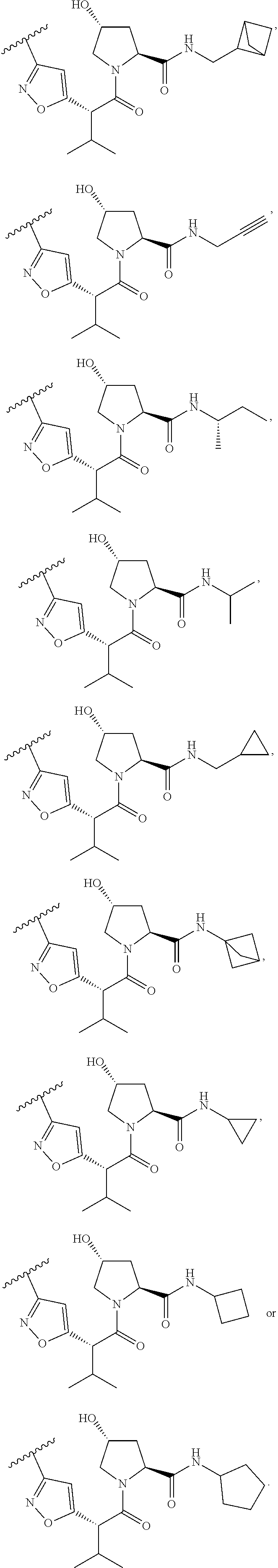

255. The compound of claim 242, or a pharmaceutically acceptable salt thereof, wherein the degradation moiety is

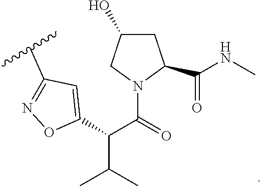 [SMILES: *c1cc([C@H](C(=O)N2C[C@H](O)C[C@H]2C(=O)NC)C(C)C)on1]

256. The compound of claim 255, or a pharmaceutically acceptable salt thereof, wherein the linker has the structure of

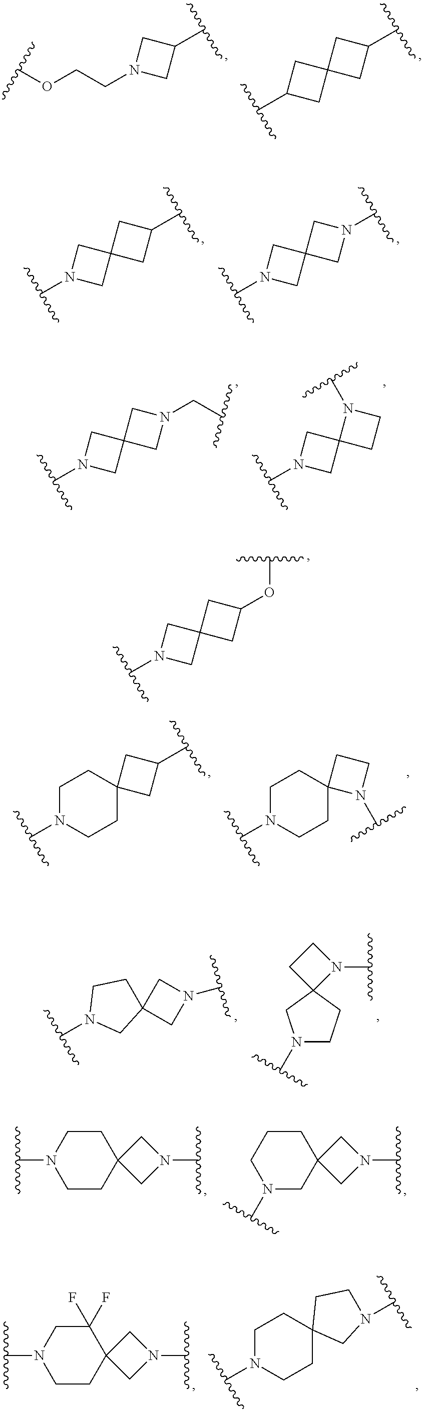

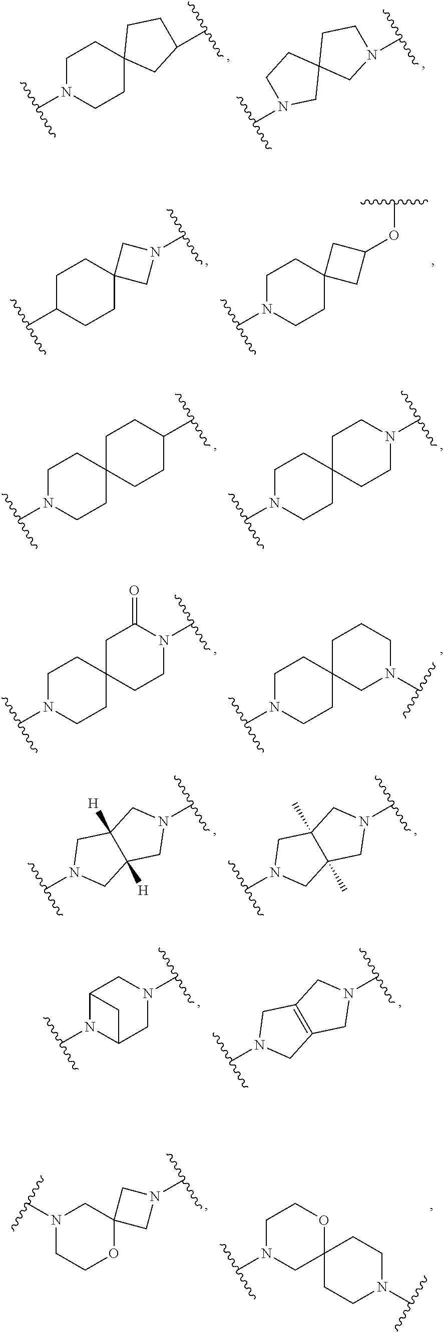

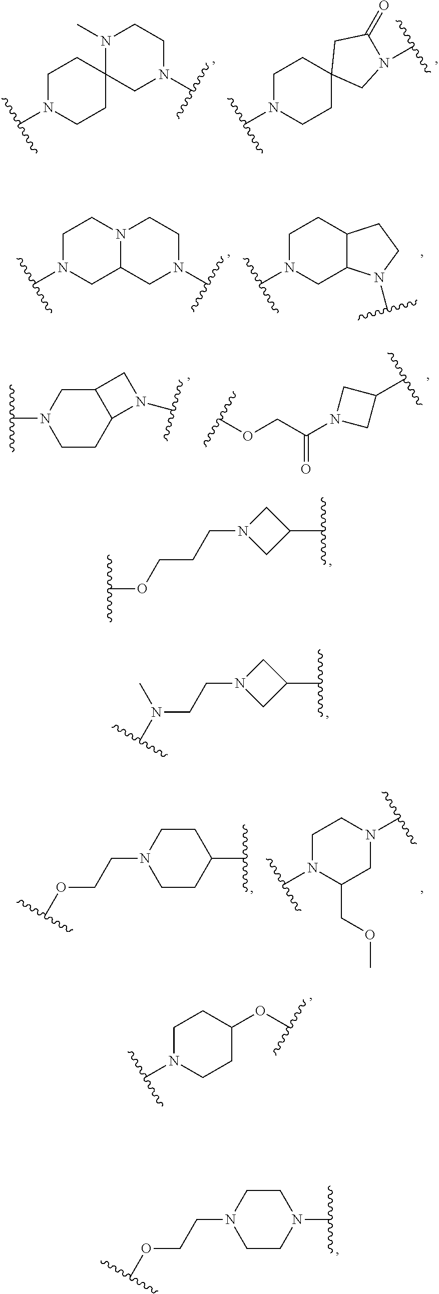

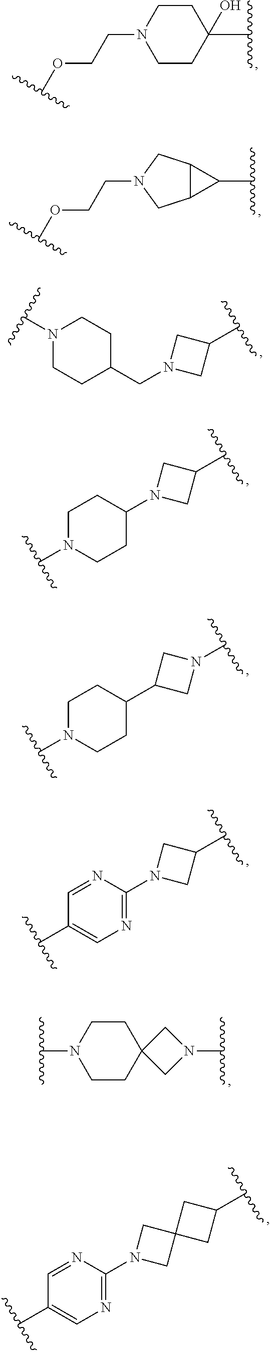

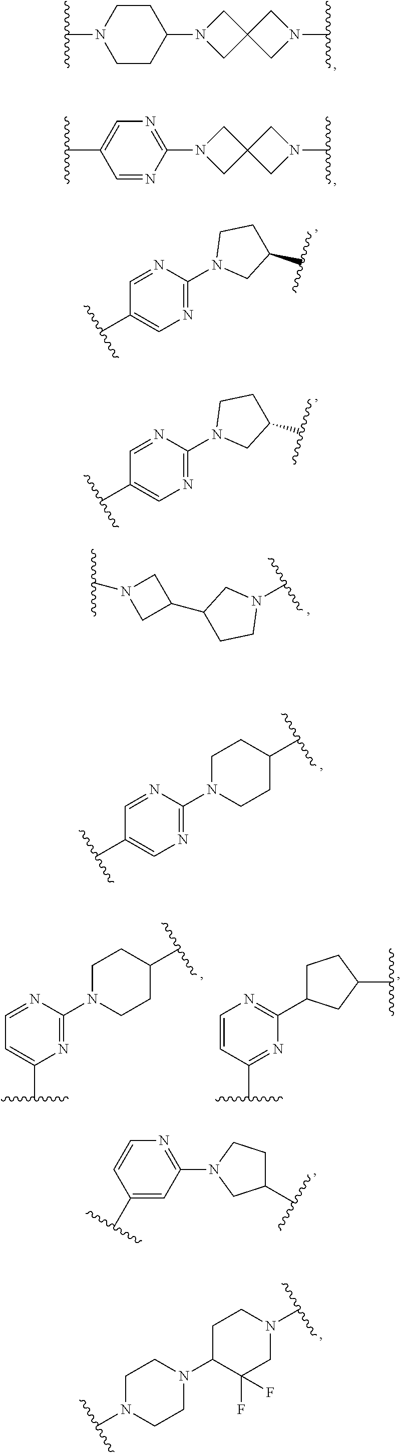

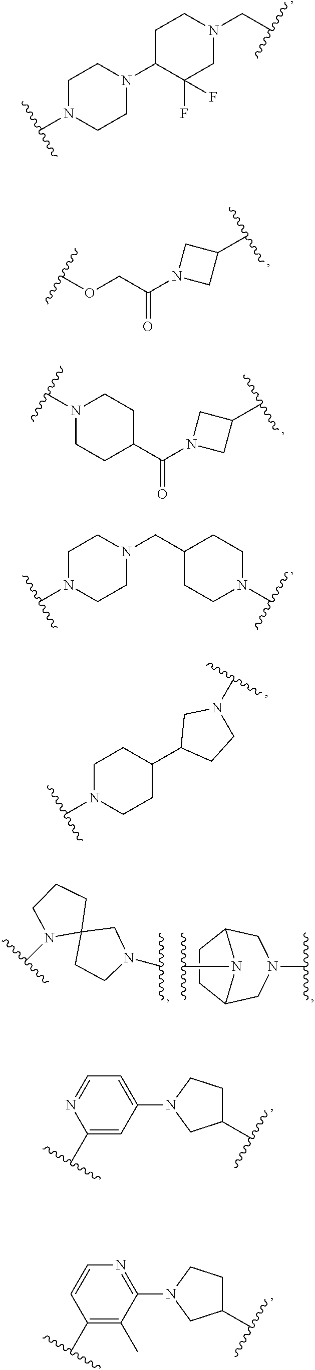

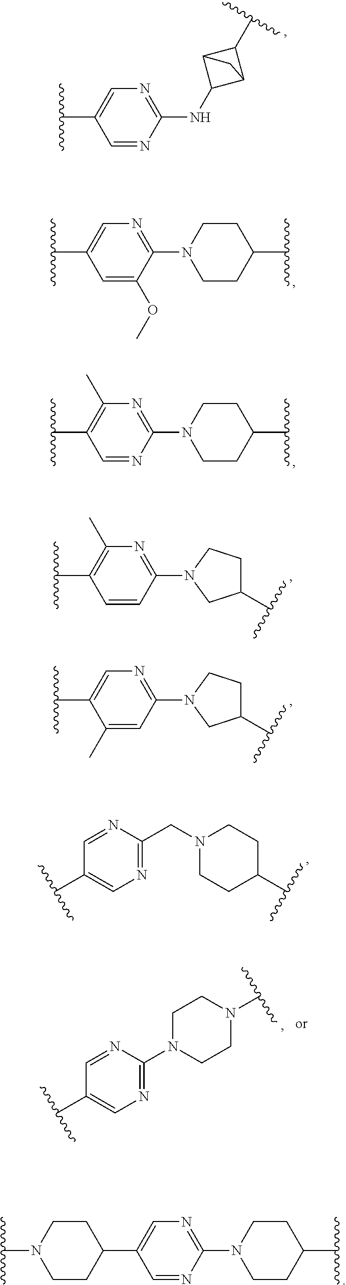

257. The compound of claim 255, or a pharmaceutically acceptable salt thereof, wherein the linker has the structure of

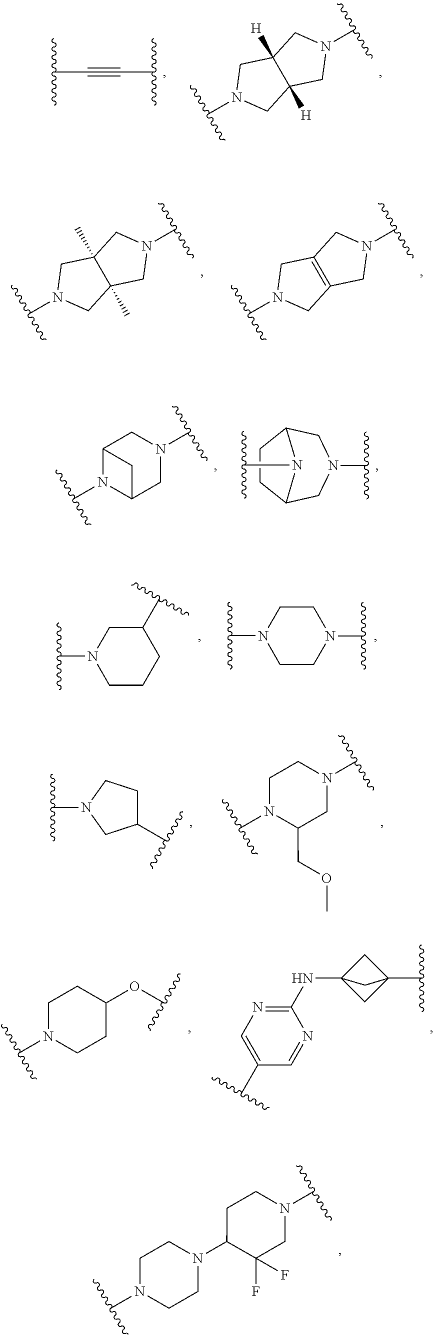

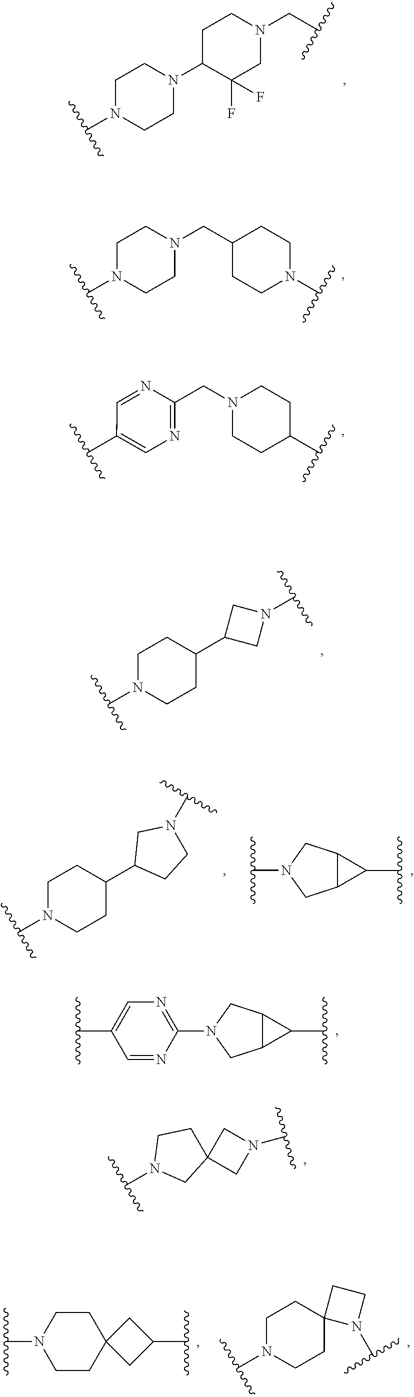

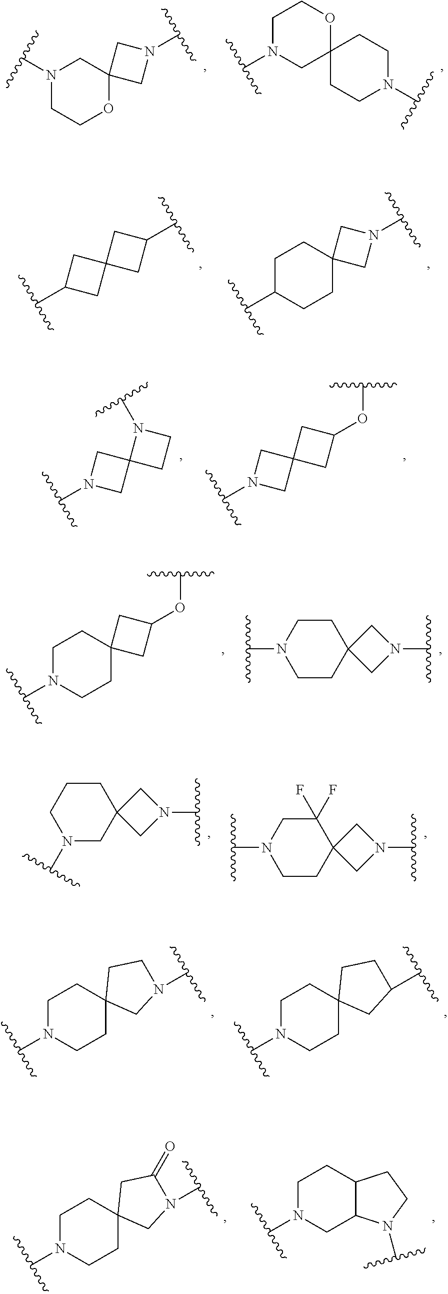

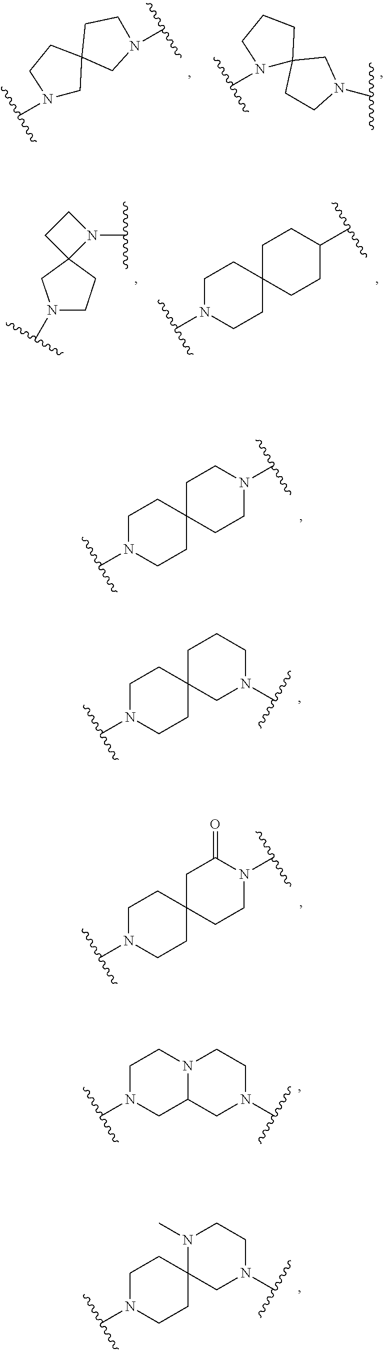

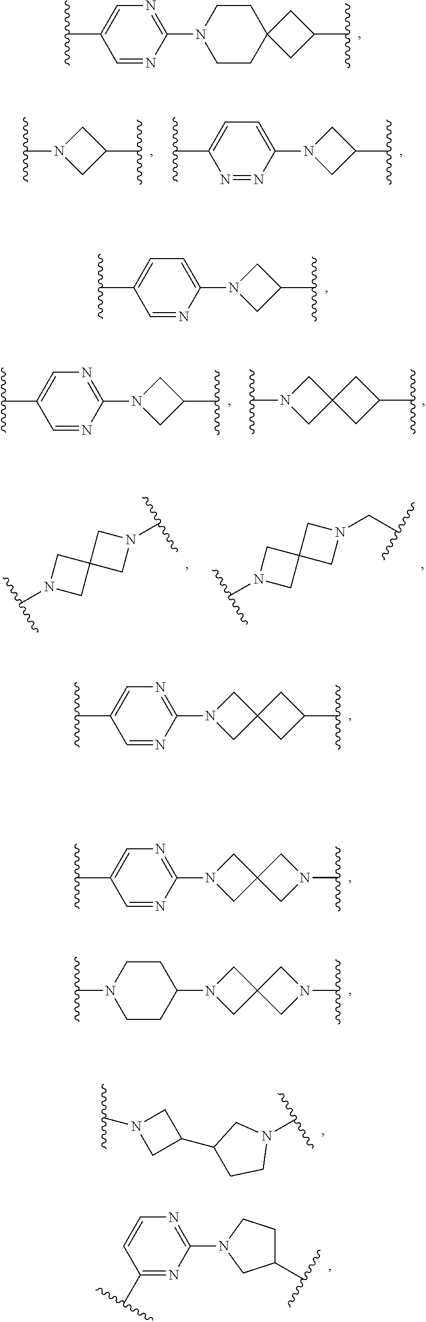

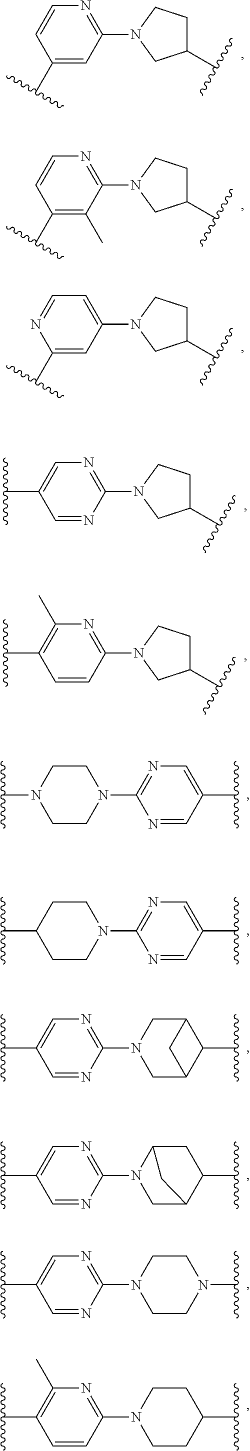

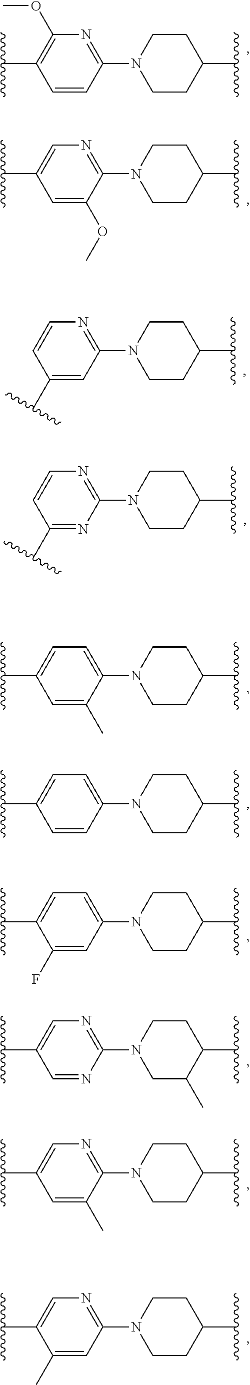

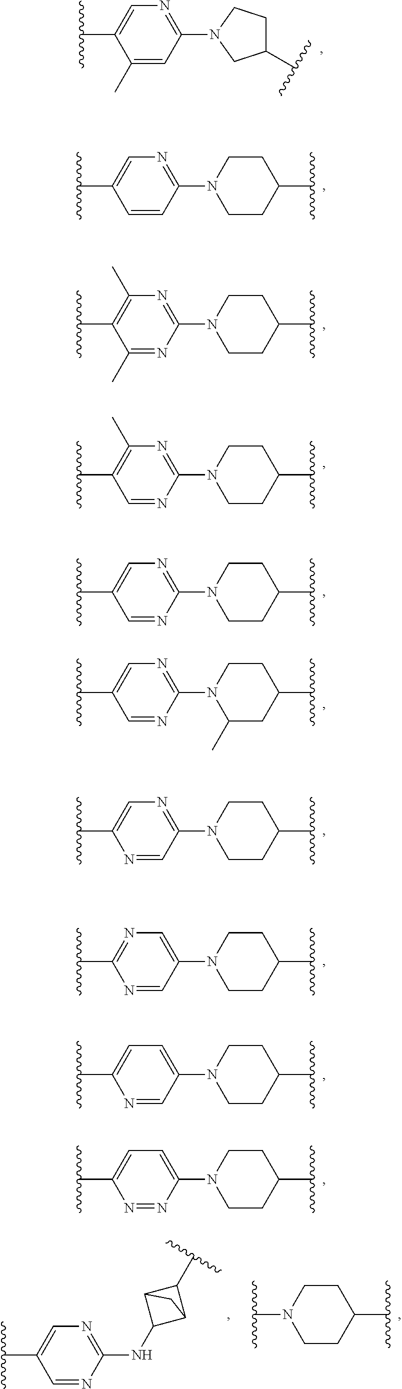

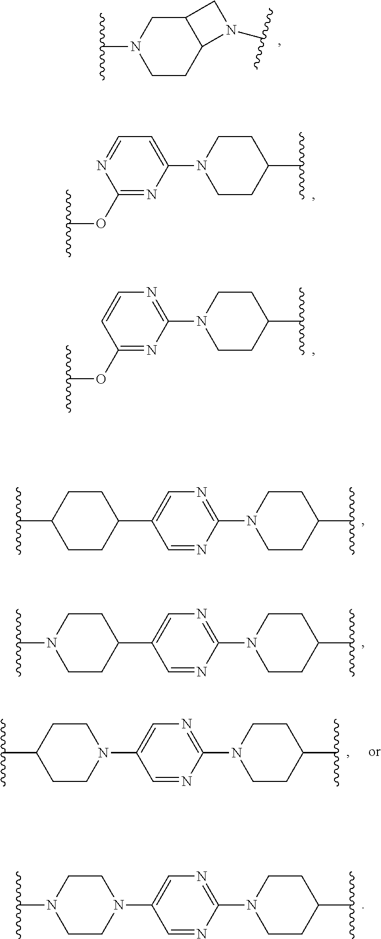

258. A compound according to claim 228 selected from the group consisting of 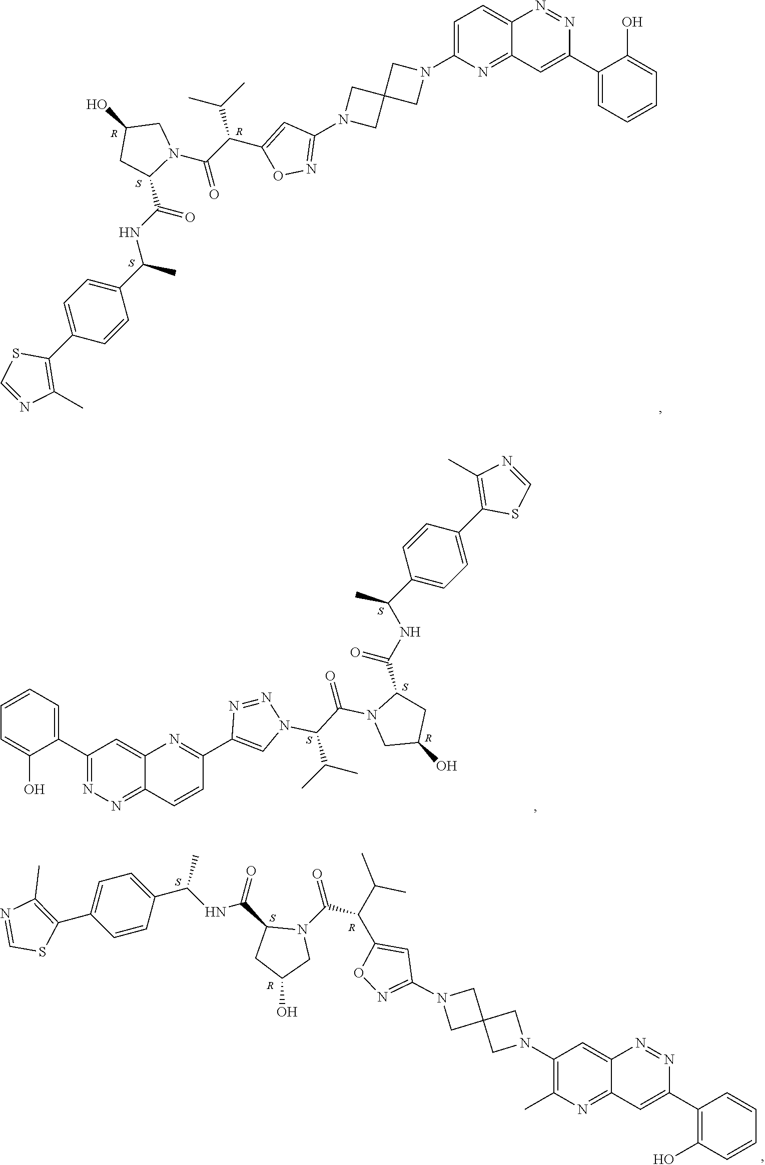 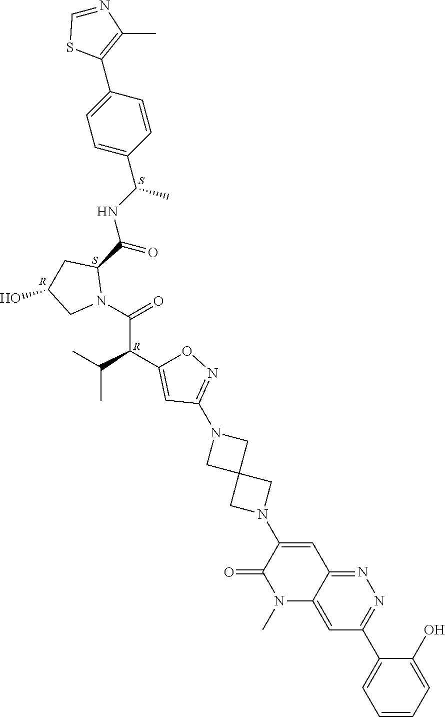
and pharmaceutically acceptable salts thereof.

259. A pharmaceutical composition comprising a compound of claim 228 and a pharmaceutically acceptable excipient.

260. A method of treating cancer comprising administering a compound of claim 228, or a pharmaceutically acceptable salt thereof, to a patient in need of such therapy.

261. The method of claim 260, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.
